SEQUENCE LISTING (51 sequences)

SEQ ID NO: 1 - DNA, 146 bp, Artificial Sequence
  primer_bind (1)..(18): Forward (Fwd)  primer binding site
tgtaaaacgacggccagtgaattgtaatacgactcactatagggcgaattggagctccaccgcggtggcggccgcataacttcgtatagcatacattatacgaagttatggatcaggccaaatcggccgagctcgaattcgtcgac

SEQ ID NO: 2 - DNA, 125 bp, Artificial Sequence
  protein_bind (1)..(6): BamHI restriction site
ggatccggccataagggcctgatccttcgagggggggcccggtaccagcttttgttccctttagtgagggttaatttcgagcttggcgtaatcatggtcatagctgtttcctgtgtgaaattgtt

SEQ ID NO: 3 - DNA, 102 bp, Artificial Sequence
  primer_bind (1)..(18): Forward (Fwd)  primer binding site
tgtaaaacgacggccagtgagcgcgcgtaatacgactcactatagggcgaattgggtaccgggccacaagtttgtacaaaaaagcaggctctcgaggtcgac

SEQ ID NO: 4 - DNA, 188 bp, Artificial Sequence
  protein_bind (1)..(6): BamHI restriction site
ggatccacccagctttcttgtacaaagtggtctagacctctcttggccgcataacttcgtatagcatacattatacgaagttatgcggccgccaccgcggtggagctccagcttttgttccctttagtgagggttaattgcgcgcttggcgtaatcatggtcatagctgtttcctgtgtgaaattgtt

SEQ ID NO: 5 - DNA, 93 bp, Artificial Sequence
  primer_bind (1)..(18): Forward (Fwd) primer binding site
tgtaaaacgacggccagtgaattgcgcgcaattaaccctcactaaagggaacaaagatgtgtaactataacggtcctaaggtagcgagtcgac

SEQ ID NO: 6 - DNA, 103 bp, Artificial Sequence
  protein_bind (1)..(6): BamHI restriction site
ggatcctgccatttcattacctctttctccgcacccgacatagatgcatcgcccctatagtgagtcgtattacatagctgtttcctggaaattgttatccgct

SEQ ID NO: 7 - DNA, 99 bp, Artificial Sequence
  primer_bind (1)..(18): Forward (Fwd) primer binding site
tgtaaaacgacggccagtgagcgcgcaattaaccctcactaaagggaacaaaagctggatcaacaagtttgtacaaaaaagcaggctctcgaggtcgac

SEQ ID NO: 8 - DNA, 117 bp, Artificial Sequence
  protein_bind (1)..(6): BamHIi restriction site
ggatccacccagctttcttgtacaaagtggttgatccaattcgccctatagtgagtcgtattacgcgcgcttggcgtaatcatggtcatagctgtttcctggaaattgttatccgct

SEQ ID NO: 9 - DNA, 30 bp, Artificial Sequence
  Description of Artificial Sequence linker/primer upper oligonucleotide
ctaggcgcgccgagagatctagagagagag

SEQ ID NO: 10 - DNA, 24 bp, Artificial Sequence
  Description of Artificial Sequence linker/primer lower oligonucleotide
ctctctctctagatctctcggcgc

SEQ ID NO: 11 - DNA, 68 bp, Artificial Sequence
  Description of Artificial Sequence amplification primer
gagagactcgaggtcgacgagagaggcccgggcggccgcgatcgcggccggccagtctttaattaact

SEQ ID NO: 12 - DNA, 63 bp, Artificial Sequence
  Description of Artificial Sequence amplification primer
gagagaggatccgagagaggccagagaggccatttaaatgcccgggctgcaggaattcgatat

SEQ ID NO: 13 - DNA, 49 bp, Artificial Sequence
  Description of Artificial Sequence amplification primer
gagagagcggccgcccgggccatttaaatccggcttactaaaagccaga

SEQ ID NO: 14 - DNA, 24 bp, Artificial Sequence
  Description of Artificial Sequence amplification reverse primer
agcggataacaatttcacacagga

SEQ ID NO: 15 - DNA, 45 bp, Artificial Sequence
  Description of Artificial Sequence amplification primer
gagagaggcctctctggccactagtctgcagactggctgtgtata

SEQ ID NO: 16 - DNA, 18 bp, Artificial Sequence
  Description of Artificial Sequence amplification forward primer
tgtaaaacgacggccagt

SEQ ID NO: 17 - DNA, 77 bp, Artificial Sequence
  Description of Artificial Sequence amplification primer comprising the loxP site
gagagaggatccagagagataacttcgtataatgtatgctatacgaagttatgagagaggccagagaggccatttaa

SEQ ID NO: 18 - DNA, 68 bp, Artificial Sequence; identical to SEQ ID NO: 11
  Description of Artificial Sequence amplification primer

SEQ ID NO: 19 - DNA, 31 bp, Artificial Sequence
  Description of Artificial Sequence linker/adapter upper oligonucleotide
gatcaggccaaatcggccgagctcgaattcg

SEQ ID NO: 20 - DNA, 29 bp, Artificial Sequence
  Description of Artificial Sequence linker/adapter lower oligonucleotide
tcgagaattcgagctcggccatttggcct

SEQ ID NO: 21 - DNA, 39 bp, Artificial Sequence
  Description of Artificial Sequence linker/adapter upper oligonucleotide
gatcaggcccttatggccggatccactagtgcggccgca

SEQ ID NO: 22 - DNA, 38 bp, Artificial Sequence
  Description of Artificial Sequence linker/adapter lower oligonucleotide
tcgatgcggccgcctagtggatccggccataagggcct

SEQ ID NO: 23 - DNA, 56 bp, Artificial Sequence
  Description of Artificial Seque ce PCR T7 Rev primer
gtgtgatatcgccctatagtgagtcgtattacatagctgtttcctgtgtgaaattg

SEQ ID NO: 24 - DNA, 70 bp, Artificial Sequence
  Description of Artificial Sequence PCR T3 Fwd primer
gagagatatctttgttccctttagtgagggttaattgcgcgcaattcactggccgtcgttttacaacgtc

SEQ ID NO: 25 - DNA, 68 bp, Artificial Sequence; identical to SEQ ID NO: 11
  Description of Artificial Sequence PCR primer

SEQ ID NO: 26 - DNA, 63 bp, Artificial Sequence; identical to SEQ ID NO: 12
  Description of Artificial Sequence PCR primer

SEQ ID NO: 27 - DNA, 59 bp, Artificial Sequence
  Description of Artificial Sequence PCR primer
gtgtaactataacggtcctaaggtagcgagtcgacgagagaggcccgggcggccgcgat

SEQ ID NO: 28 - DNA, 67 bp, Artificial Sequence
  Description of Artificial Sequence PCR primer
gcatctatgtcgggtgcggagaaagaggtaatgaaatggcaggatccgagagaggccagagaggcca

SEQ ID NO: 29 - DNA, 69 bp, Artificial Sequence
  Description of Artificial Sequence PCR primer
gagagtctagataacttcgtatagcatacattatacgaagttataaatcaatctaaagtatatatgagt

SEQ ID NO: 30 - DNA, 69 bp, Artificial Sequence
  Description of Artificial Sequence PCR primer
gagagtctagataacttcgtataatgtatgctatacgaagttataaaacttcatttttaatttaaaagg

SEQ ID NO: 31 - DNA, 76 bp, Artificial Sequence
  Description of Artificial Sequence AttB1 linker upper oligonucleotide
cgggccacaagtttgtacaaaaaagcaggctctcgaggtcgacgagaggccagagaggccggccgagattaattaa

SEQ ID NO: 32 - DNA, 80 bp, Artificial Sequence
  Description of Artificial Sequence AttB1 linker lower oligonucleoide
ttaattaatctcggccggcctctctggcctctcgtcgacctcgagagcctgcttttttgtacaaacttgtggcccggtac

SEQ ID NO: 33 - DNA, 78 bp, Artificial Sequence
  Description of Artificial Sequence AttB2 linker upper oligonucleotide
ggccatgacggccgagagatttaaatgagagaggatccacccagctttcttgtacaaagtggtctagacctctcttgg

SEQ ID NO: 34 - DNA, 72 bp, Artificial Sequence
  Description of Artificial Sequence AttB2 linker lower oligonucleotide
gaggtctagaccactttgtacaagaaagctgggtggatcctctctcatttaaatctcttggccgtcatggcc

SEQ ID NO: 35 - DNA, 40 bp, Artificial Sequence
  Description of Artificial Sequence LoxP linker upper oligonucleotide
ccgcataacttcgtatagcatacattatacgaagttatgc

SEQ ID NO: 36 - DNA, 50 bp, Artificial Sequence
  Description of Artificial Sequence LoxP linker lower oligonucleotide
ggccgcataacttcgtataatgtatgctatacgaagttatgcggccaaga

SEQ ID NO: 37 - DNA, 11 bp, Artificial Sequence
  Description of Artificial Sequence plasmid junction linker upper oligonucleotide
ggccatgagat

SEQ ID NO: 38 - DNA, 11 bp, Artificial Sequence
  Description of Artificial Sequence Plasmid junction linker lower oligonucleotide
ctagatctcat

SEQ ID NO: 39 - DNA, 93 bp, Artificial Sequence
  Description of Artificial Sequence I-CeuI/PI-SceI adaptor oligonucleotide (up adaptor strand)
cgcgctaactataacggtcctaaggtagcgagtcgacgagagagagaggatccatctatgtcgggtgcggagaaagaggtaatgaaatggcag

SEQ ID NO: 40 - DNA, 93 bp, Artificial Sequence
  Description of Artificial Sequence I-CeuI/PI-SceI adaptor oligonucleotide (down adaptor strand)
cgcgctgccatttcattacctctttctccgcacccgacatagatggatccgagagagagagtcgactcgctaccttaggaccgttatagttag

SEQ ID NO: 41 - DNA, 69 bp, Artificial Sequence; identical to SEQ ID NO: 29
  Description of Artificial Sequence XbaI - LoxP Tag primer 3F

SEQ ID NO: 42 - DNA, 69 bp, Artificial Sequence; identical to SEQ ID NO: 30
  Description of Artificial Sequence XbaI - LoxP Tag primer 3R

SEQ ID NO: 43 - DNA, 520 bp, Artificial Sequence
  Description of the artificial sequence oriV sequence
ccggcgttgtggataccacgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgatgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctggcgcgtgaccgcgcacgccgaaggggggtgcccccccttctcgaaccctcccgg

SEQ ID NO: 44 - DNA, 34 bp, Artificial Sequence
  Description of the artificial sequence yeast FRT element
gaagttcctattctctagaaagtataggaacttc

SEQ ID NO: 45 - DNA, 12 bp, Artificial Sequence
  Description of Artificial Sequence double strand oligonucleotide adaptor upper strand phosphorylated at the 5′ end
tcgaagcttccg

SEQ ID NO: 46 - DNA, 12 bp, Artificial Sequence
  Description of Artificial Sequence double strand oligonucleotide adaptor lower strand
cgcgcggaagct

SEQ ID NO: 47 - DNA, 70 bp, Artificial Sequence
  Description of Artificial Sequence PCR primer
agagagagagatctagaataacttcgtataatgtatgctatacgaagttatctgtcaaacatgagaattg

SEQ ID NO: 48 - DNA, 67 bp, Artificial Sequence
  Description of Artificial Sequence PCR primer
gagagagagatctagataacttcgtatagcatacattatacgaagttatcgaatttctgccattcat

SEQ ID NO: 49 - DNA, 24 bp, Artificial Sequence
  Description of Artificial Sequence Sau3AI/XbaI adaptor upper strand
gagagagagatctagaaagctcca

SEQ ID NO: 50 - DNA, 12 bp, Artificial Sequence
  Description of Artificial Sequence Sau3AI/XbaI adaptor lower strand
gatctggagctt

SEQ ID NO: 51 - DNA, 3003 bp, Artificial Sequence
  Description of Artificial Sequence pFLC-II
ggatccacccagctttcttgtacaaagtggtctagacctctcttggccgcataacttcgtatagcatacattatacgaagttatgcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccacaagtttgtacaaaaaagcaggctctcgag

(19) United States
(12) Patent Application Publication
(10) Pub. No.: US 2005/0090010 A1
(43) Pub. Date: Apr. 28, 2005

(54) Cloning vectors and method for molecular cloning

(76) Inventors: Yoshihide Hayashizaki, Ibaraki (JP); Piero Carninci, Ibaraki (JP)

Correspondence Address: BIRCH STEWART KOLASCH & BIRCH, PO BOX 747, FALLS CHURCH, VA, 22040-0747, US

(21) Appl. No.: 10/469,508

(22) PCT Filed: Feb. 25, 2002
(86) PCT No.: PCT/JP02/01667

(30) Foreign Application Priority Data
Mar. 2, 2001 (JP) 2001-57794

(51) Int. Cl. C12P 19/34; C12N 7/00; C12N 15/74

(52) U.S. Cl. 435/472; 435/235.1

(57) ABSTRACT

The invention discloses a family of cloning vectors capable of cloning nucleic acid inserts of interest of long sizes, with low or reduced background and high efficiency of excision and method for preparing these vectors and library thereof. As example, it is disclosed a cloning vector comprising a construction vector segment (CS) and a replaceable segment (RS), wherein the size of CS is: 36.5 kb≦CS<38 kb, preferably CS is 37.5 kb, comprising lox recombination sites for Cre-recombination and/or att recombination sites for Gateway-like recombination, preferably also a background-reducing system selected from the group of: the ccdB gene, a lox sequence, the lacZ gene, and asymmetric site sequences recognized by restriction endonucleases.

### FIELD OF THE INVENTION

The present invention relates to recombinant DNA technology. In particular, it is disclosed a novel cloning vector family and in vitro and in vivo method for cloning of nucleic acids of interest.

### BACKGROUND ART

Efficient genomic and cDNA cloning vectors are important tools in molecular genetic research, because high quality, representative libraries are rich sources for the analysis of many genes.

Full-length cDNAs are the starting material for the construction of the full-length libraries (for example, the RIKEN mouse cDNA encyclopedia, RIKEN and Fantom Consortium, “Functional annotation of a full-length mouse cDNA collection”, Nature, Feb. 8, 2001, Vol.409:685-690). In contrast to standard cloning techniques, full-length cDNA cloning has the inherent risk of under representation or absence of long clones from the libraries, and cDNAs deriving from very long mRNAs are not cloned if the capacity of the vector is not sufficient.

Available plasmid cloning vectors show bias for short cDNAs: shorter fragments are cloned more efficiently than longer ones when competing during ligation and library amplification steps. Although plasmid electroporation does not show relevant size bias, during circularization of plasmid molecules in the ligation step, in a mixed ligation reaction, short cDNAs are ligated more efficiently than longer cDNAs (Sambrook et al., 1989, Cold Spring Harbor Laboratory Press, Molecular Cloning, NY, USA). Cloning vectors derived from bacteriophage have been disclosed as particularly useful for cloning, propagation of DNAs and for library construction. Ligated mixtures of insert and bacteriophage vector DNAs can be efficiently packaged in vitro and introduced into bacteria by infection.

Bacteriophage vectors allow cloning of cDNAs sequences, however, the final product for large-scale sequencing should be a plasmid for large-scale colony picking, propagation, DNA preparation and sequencing reactions (Shibata et al., 2000, Genome Res. 10: 1757-1771).

Cloning vectors for automatic plasmid excision should have a capacity for wide-range cDNA cloning, that is including cDNAs as short as 0.5 Kb and as long as 15 Kb, which are visible on agarose gel when using trehalose during the first strand cDNA synthesis (Carninci et al., 1998, Proc. Natl. Sci. USA, 95:520-524).

There are a number of bacteriophage vectors allowing whole library bulk excision, but they are not optimal in terms of cloning size or bulk excision protocol.

Examples of plasmid excision from bacteriophage vector having a cloned insert were obtained with the λ-Zap II (Short et al., 1988, Nucl. Acids Res.,16:7853-7600). However, the bulk excision from λ-Zap II shows size bias towards short inserts when using a mixed sample like a cDNA library, which contains both short and long clones. Using λ-Zap II, long and rare cDNAs are difficult to obtain.

Other vectors designed for cDNA cloning and plasmid excision like the λ-Lox derivatives (Palazzolo M. et al., 1990, Gene, 88: 25-36), λ-YES (Elledge et al., 1991, Proc. Natl. Acad. Sci. USA., 88: 1731-5) and λ-Triplex™ (CLONTECHniques, January 1996), accept cDNAs that do not exceed 9˜10 Kb. Alternatively, vectors for genomic libraries construction and Cre-lox mediated plasmid excision accept inserts longer than 7 Kbp, such as λ PS (Nehls et al., 1994a, Biotechniques, 17: 770-775), λpAn (Holt et al., 1993, Gene, 133: 95-97), λGET (Nehls et al., 1994b, Oncogene, 9: 2169-2175), λ-MGU2 (Maruyama and Brenner, 1992, Gene, 120: 135-141) and a vector based on Tn1721 excision system, λRES (Altenbucher, J, 1993, Gene, 123: 63-68). However, these vectors do not allow the preparation of wide range size cDNA libraries.

Only among the λSK series there were some vectors with calculated capacity between 0.2 to 15.4 Kb (Zabarovski et al., 1993, Gene, 127: 1-14), which would be suitable for wide-range size cDNA cloning purpose. Unfortunately, the rudimental excision system of λSK is based on simple restriction digestion, which causes internal cleavage of cDNA clones and probably this is the reason why these vectors are not commonly used for cDNA cloning.

Japanese patent application having publication number P2000-325080A, discloses a modified λ PS vector. The new vector, indicated with the term λ-FLC-1, comprised a 6 kb nucleic acid sequence (stuffer II) in the left arm of the λ PS vector so that the size of the vector, without considering the cDNA of interest, was 38 kb. This modified λ PS vector was described as being able to insert broad range size of cDNAs.

The λ-FLC-1, even if useful for generic (or “standard”) large size cDNA libraries, still shows a bias for short and not full-length cDNAs, so that very long, rare and important full-length cDNAs are difficult to obtain, in particular, in case of strongly normalized and/or subtracted cDNA libraries.

A further problem in the art refers to the efficiency of bulk excision recombination mechanism.

Bulk cDNAs (cDNA library), that is a library of cDNA comprising a wide range size of cDNAs, short, medium and long ones, are inserted in cloning vectors. These inserts are then transferred in other functional or specialized vectors that have desired characteristics, such as expression vectors. This transfer is called subcloning. The functional or specialized vectors used for subcloning DNA segments are functionally diverse. These include but are not limited to: vectors for expressing genes in various organisms; for regulating gene expression; for providing tags to aid in protein purification or to allow tracking of proteins in cells; for modifying the cloned DNA segment (e.g., generating deletions); for the synthesis of probes (e.g, riboprobes); for the preparation of templates for DNA sequencing; for the identification of protein coding regions; for the fusion of various protein-coding regions; to provide large amounts of the DNA of interest, etc. It is common that a particular investigation will involve subcloning the DNA segment of interest into several different specialized vectors.

Traditional subcloning methods, using restriction enzymes and ligase, are time consuming and relatively unreliable.

The use of recombinase recognition systems using specific recombinase recognition sequences have been proposed and they are known as Cre-lox (Palazzolo et al., 1990, Gene, 88: 25-36) and Gateway™ (Life Technologies Catalogue; Walhout A. J. M., et al., 2000, Methods in enzymology, Vol.328: 575-592; and U.S. Pat. No. 5,888,732).

The Cre-recombinase solid-phase in vivo excision requires infection of the amplified cDNA library into a bacterial strain, which constitutively express the Cre-recombinase, for instance BNN132 (Elledge et al., 1991, Proc. Natl. Acad. Sci. USA., 88: 1731-5). However, this is not recommended because of low plasmid yield (Palazzolo et al., 1990, as above) and plasmid instability (Summers et al., 1984, Cell, 36: 1097-1103): in fact, Cre-recombinase is constitutively expressed causing formation of plasmid dimers/multimers leading to high proportion of plasmid-free cells (Summers et al., 1984, as above), impairing the sequencing efficiency.

The Gateway excision is an alternative system to the Cre-lox excision. According to the general Gateway™ system, an insert donor vector carrying a DNA of interest (insert) and a pair of recombinant sites different from each other, recombines with a donor vector comprising a subcloning vector and a pair of recombinant sites different from each other, but able to recombine with the insert donor vector recombination sites. The final product is a subclone product carrying the DNA of interest (insert) and a byproduct. The recombinant sites are attB, attP, attL and attR.

However, the Gateway™ system shows a bias for short cDNA; long cDNAs are obtained with low efficiency (Michael A. Brasch, slide “Gateway cloning of attB-PCR products”, GIBCOBRL® Technical Seminar, “Gateway Cloning Technology”, Life Technologies™, 1999).

Another further problem in the cloning system consists in the presence of background, which is due to environmental DNA contamination and to subcloning process byproducts, that is a non recombinant plasmids (plasmids without the DNA of interest).

It is instead highly desirable having a background-cutting cloning system, able to eliminate completely or having a little background.

Some background-cutting strategies have been proposed in the art. Walhout et al. (as above), for example, reports that the Gateway™ vectors, attP1-attP and attR1-attR2, also contain between the att sites the ccdB gene (Bernard P. and Couturier M., 1992, J. Mol. Biol., 226:735-746), whose protein product interferes with DNA gyrase. After recombination, only the plasmids that have lost the ccdB gene (and which are recombinant) can grow in E.coli strains not mutated for gyrA, therefore providing a selective advantage.

Plasmids carrying the gene ccdB can propagate only in specific E.coli strain, DB3.1, which carries a mutation in gyrA gene conferring resistance to ccdB (Walhout et al., as above). Therefore, this kind of recombination is limited to plasmids, since other vectors for instance λ substitution vectors used in cloning systems cannot grow and replicate in cells like DB3.1, which miss the recA protein (the recA product is required for the growth of substitution-type bacteriophage λ: Sambrook et al., 1989).

In conclusion, there is the need in his field of the art of providing of vectors having the characteristics of: i) being size bias free and allowing the preparation of “size balanced” comprising very long, rare full-length cDNAs; ii) capable of improved recombination mechanism; and iii) able of background cutting.

The cloning vectors available in the state of the art, fail to satisfy the above characteristics.

The invention disclosed in the present application is addressed to solve the problems in the art.

### SUMMARY OF THE INVENTION

The present inventors provide a new family of vectors capable of cloning nucleic acids of wide range size and preferably very long ones, with high efficiency of excision and reduced background and contamination. Also provided are methods of cloning and for preparing bulk library using such vectors.

According to a first embodiment, the invention provides a cloning vector comprising a construction vector segment (CS) and a replaceable segment (RS), wherein the size of CS is: 36.5 kb≦CS<38 kb, preferably CS is 37.5 kb. The construction vector segment preferably is made or comprise a bacteriophage λ vector fragment. The replaceable vector segment (RS) represents the segment, which is replaced by the nucleic acid insert of interest, which one intends to clone.

It has been surprisingly found that a cloning vector with this size is capable of preferably inserting cDNA of very long sizes, and it is therefore particularly advantageous for cloning very full-length cDNAs. This vector overcomes the problem in the art of existing vector λ-FLC having a construction vector segment of 38 kb, which showed a strong bias for short size cDNAs (see Table1).

The selection of a particular advantageous size of the vector for the preparation of full-length cDNAs libraries can also be applied to bacteriophage other than λ. Accordingly, the present invention also relates to a cloning bacteriophage vector comprising a construction segment (CS) and a replaceable segment (RS), wherein the size of CS is: X−1.2 kb≦CS<Xkb; X (expressed in kb) corresponding to the minimum size necessary to the bacteriophage vector for undergoing packaging. The size of CS is preferably: X−0.2 kb.

The present invention also relates to a bacteriophage vector, preferably a λ, comprising a bacterial artificial chromosome (pBAC) or a segment thereof comprising at least an origin of replication (ori). This vector can also comprise: a site into which a DNA fragment can be cloned; and a pair of inducible excision-mediating sites defining an excisable fragment that comprises the site into which the DNA fragment can be cloned. The pair of excision-mediating sites are preferably FRT sites.

This vector may further comprise an inducible origin of replication, preferably oriV.

The cloning vectors according to the invention are capable of carrying out plasmid or nucleic acid insert excision using known recombination systems, for example the Cre-lox and/or Gateway™ system.

The vectors of the invention can also comprise a background-reducing system, as ccdB gene, a lox sequence or the lacZ gene or asymmetric site sequences recognized by restriction endonuclease.

The invention also relates to cloning method using the above vectors. According to another embodiment, the invention relates to a system for reducing background or contamination by providing a cloning vector comprising a background-reducing sequence like ccdB gene and/or a lox sequence comprised into RS segment of the vector of the invention, or in case of the Gateway™ system into the RS segment of a destination or receiving vector. RS of phage or plasmid vectors can also be flanked by two asymmetric site sequences recognized by restriction endonuclease.

The invention also relates to a method for reducing background or contamination by using these vectors.

The invention also relates to methods for efficient excision of plasmid or nucleic acid of interest providing improved Cre-recombinase or Gateway™ system using the vectors according to the invention.

Preferably, the present invention relates to method for the preparation of bulk of long or full-length cDNA libraries, by using the vectors according to the invention.

The present invention also relates to a kit comprising at least a cloning vector or at least a library of vectors according to the invention.

The present invention further relates to a method for preparing at least a normalized and/or subtracted library comprising using a plasmid vector obtained with the excision method according to the invention or destination vector according to the invention, preferably reduced at single strand, as normalization and/or subtraction driver.

### BRIEF DESCRIPTION OF THE DRAWINGS

FIG. 1 is a general scheme of the vector family according to the invention. The following functional elements (not in scale) are produced in this work. In FIG. 1(a), the functional elements of the vector construction segment (CS) are: the left and right arms; the cloning size regulator (or stuffer II); a plasmid derivative of pBluescript; and the bulk excision elements (recombination sites) loxP; the size of the construction segment (CS) is between 32 and 38.3 kb. The replaceable vector segment (indicated as stuffer I or RS) is flanked by the excision Gateway™ elements (attB1 and attB2); this is the segment that will be replaced by the cDNA.

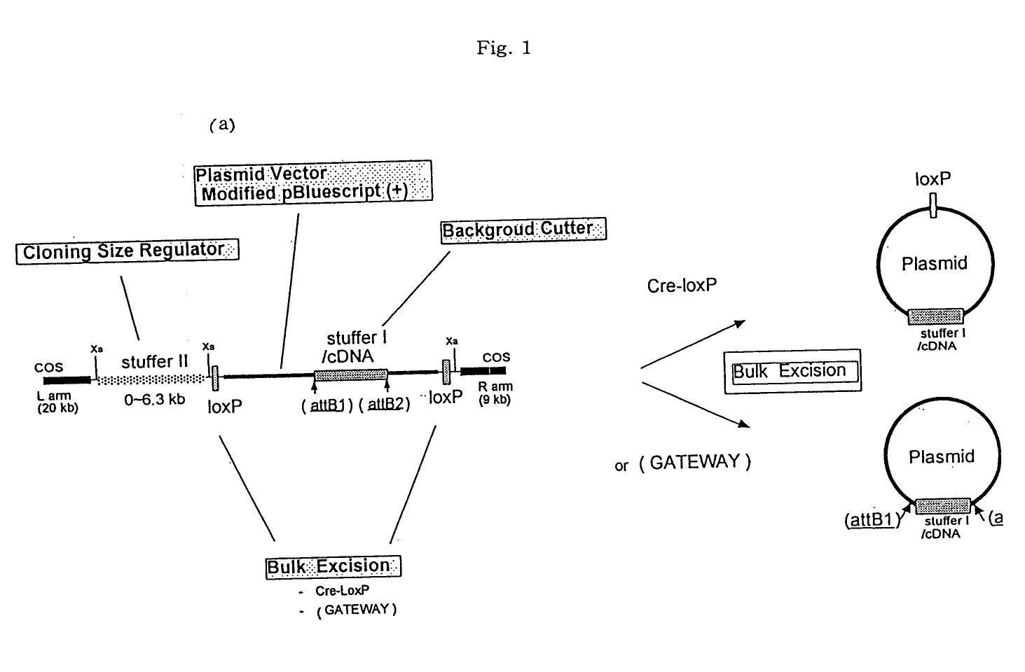

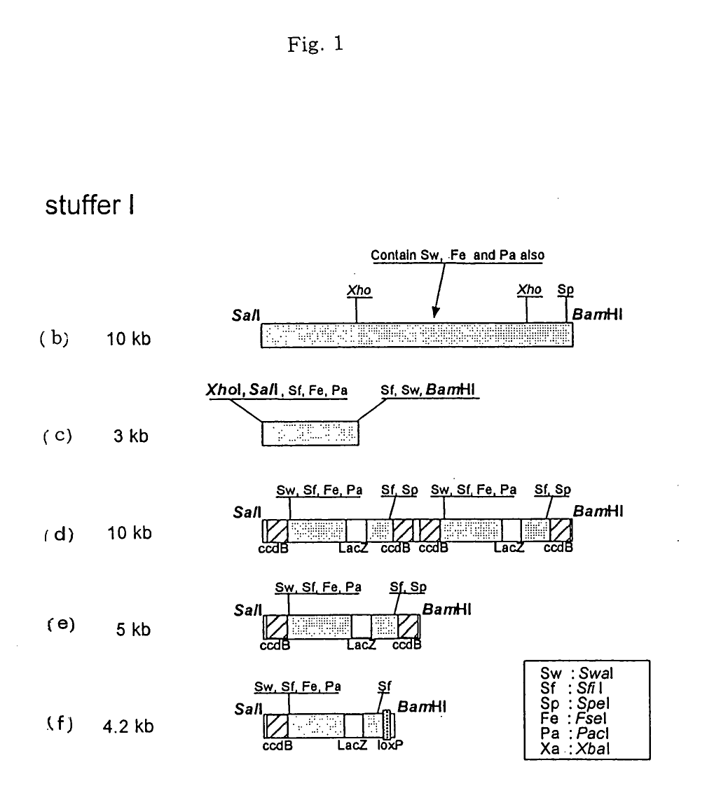

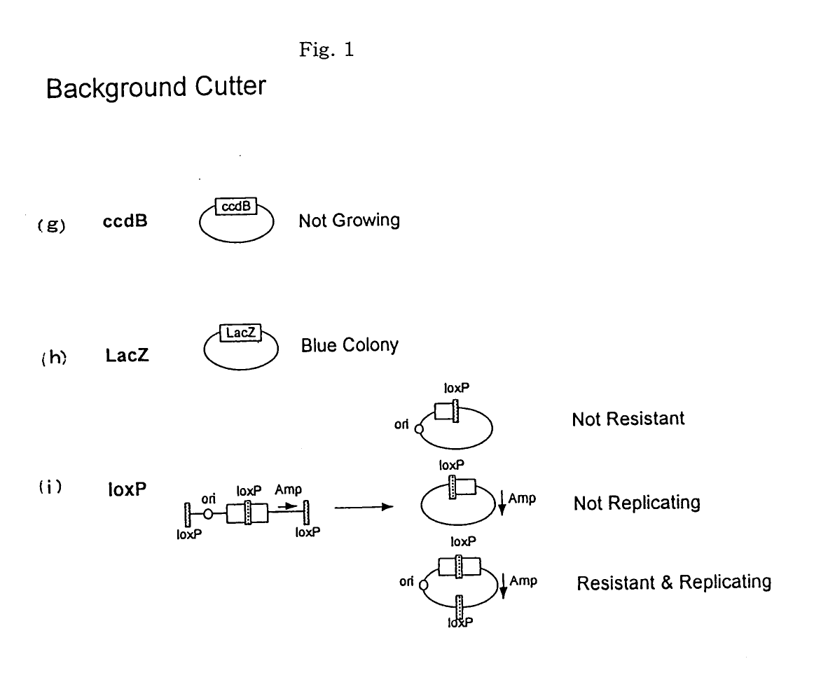

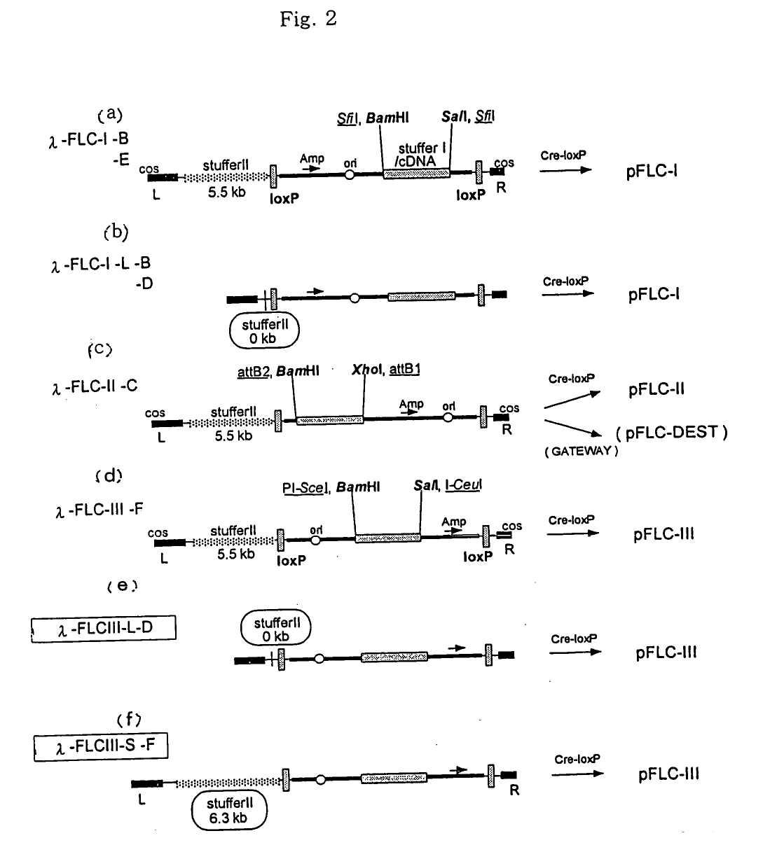

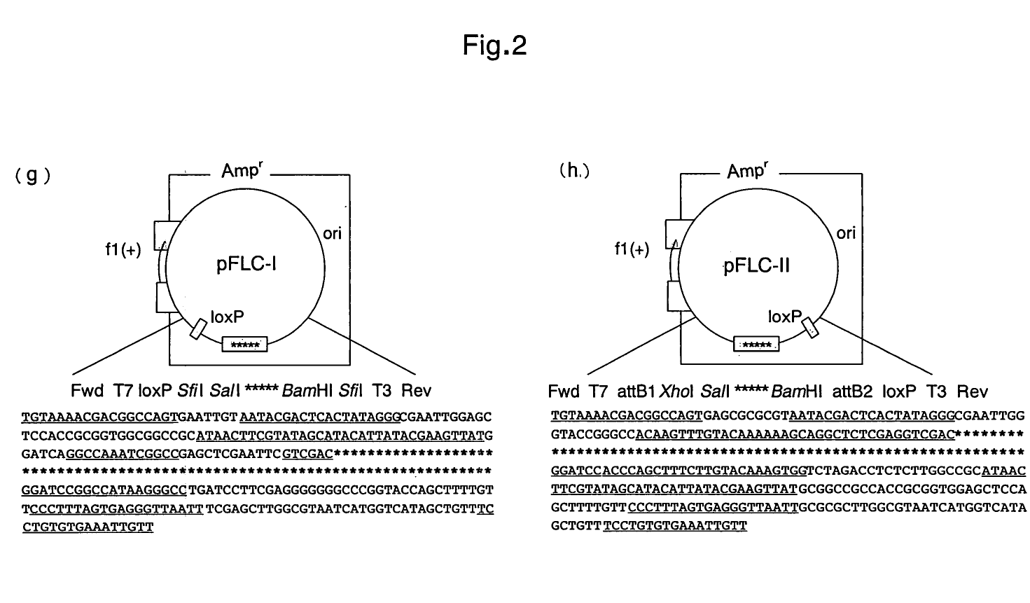

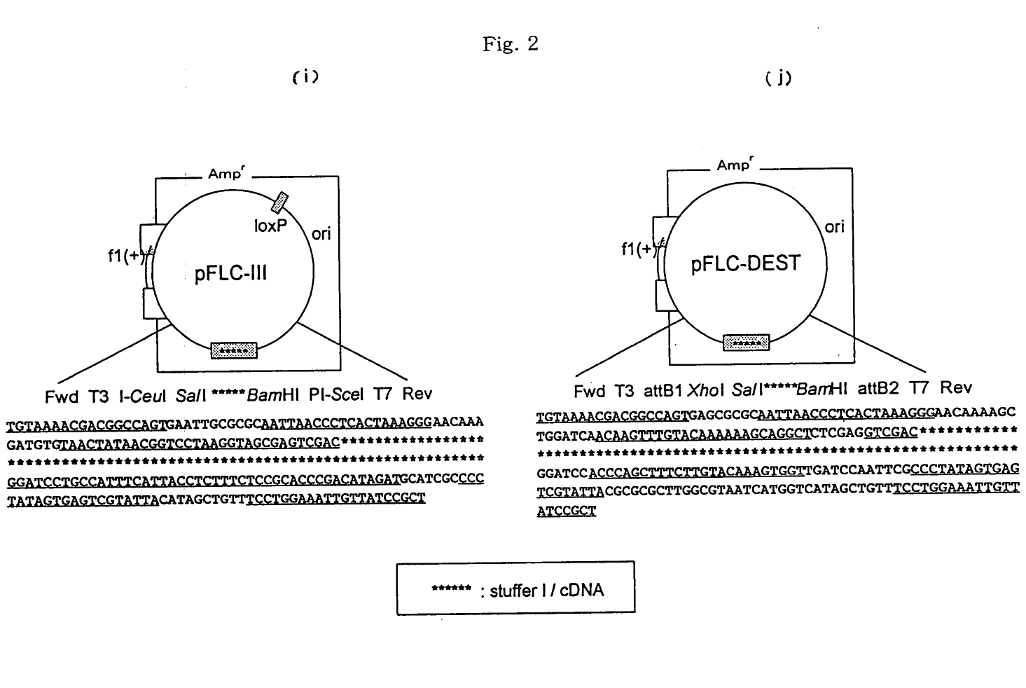

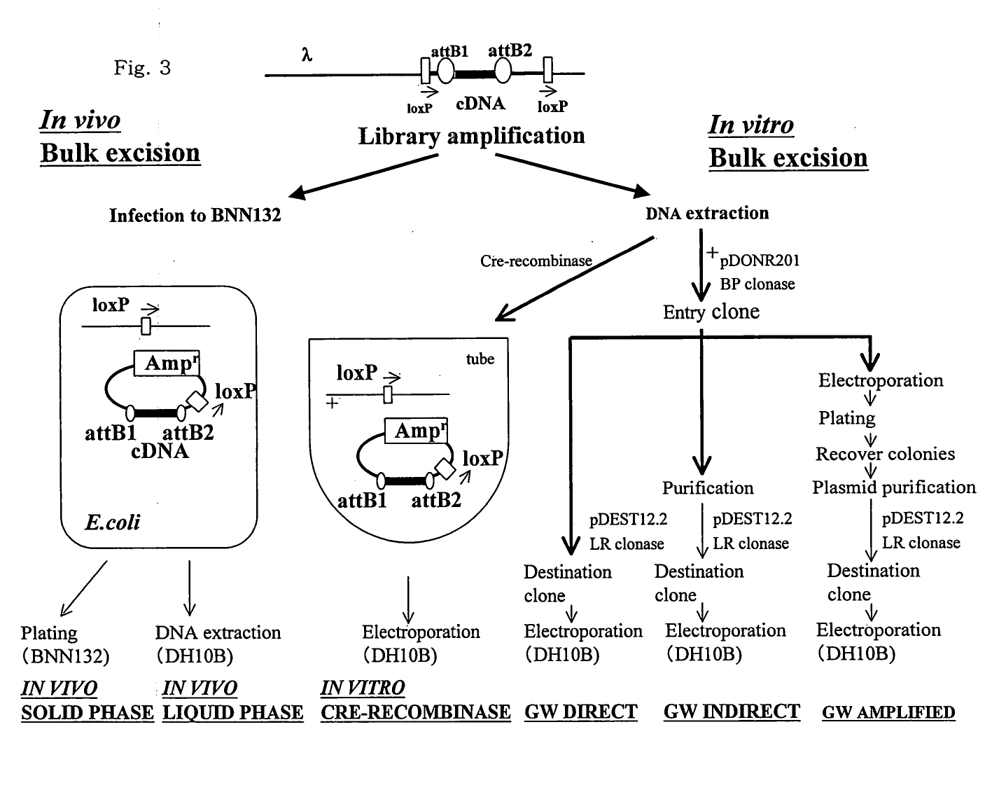

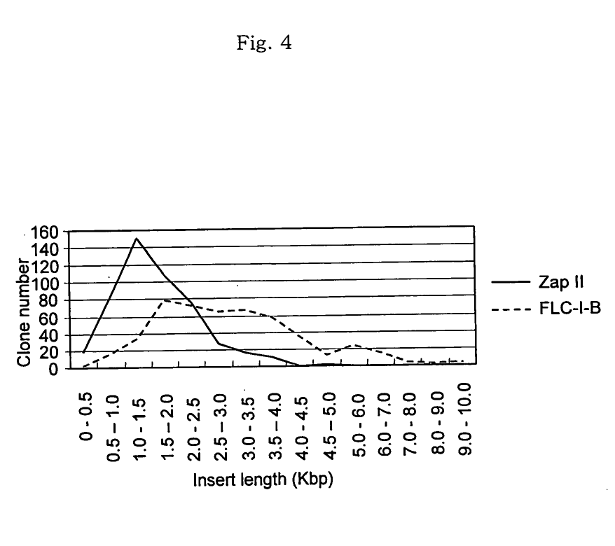

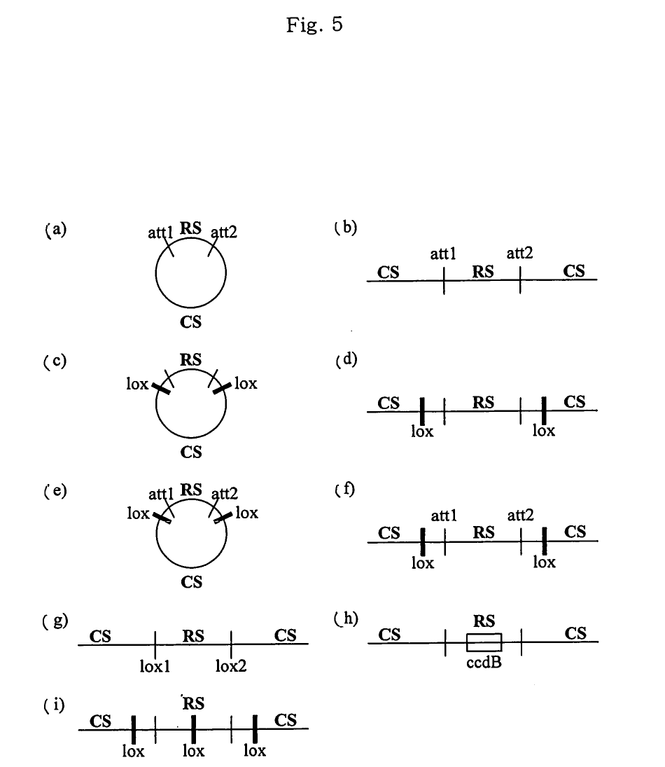

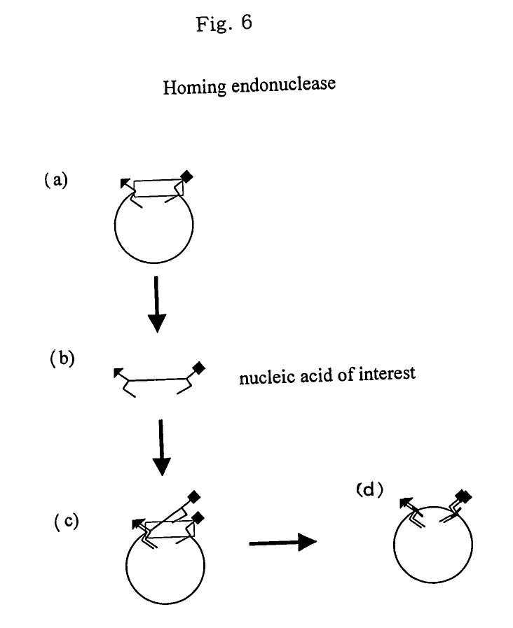

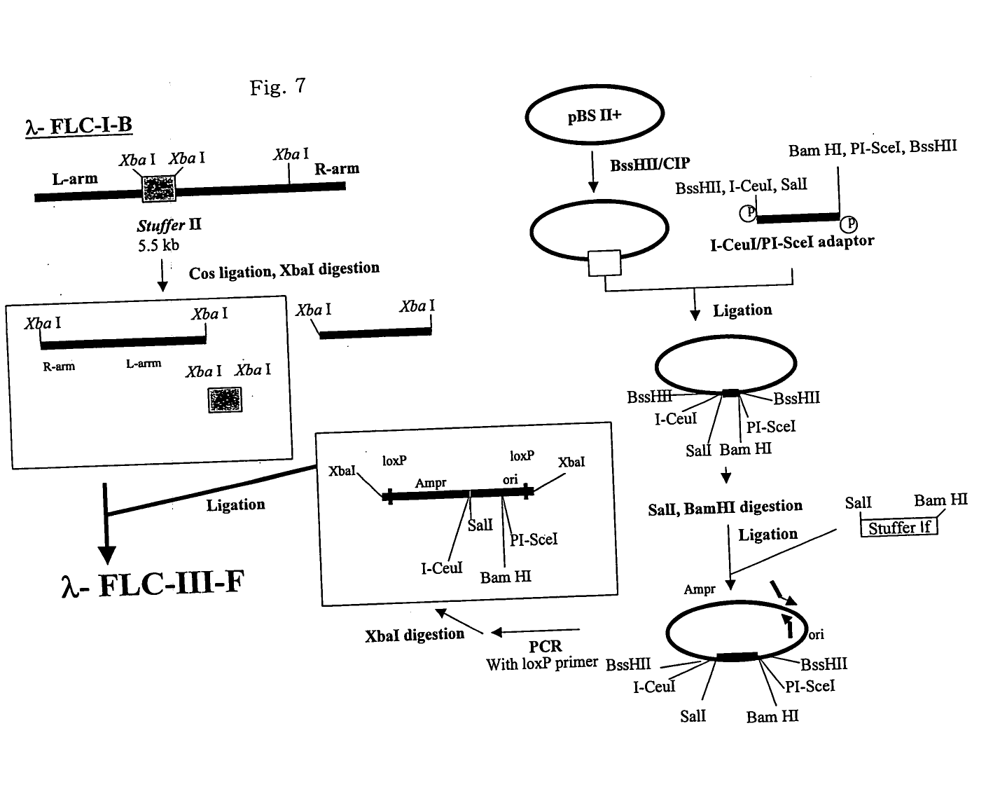

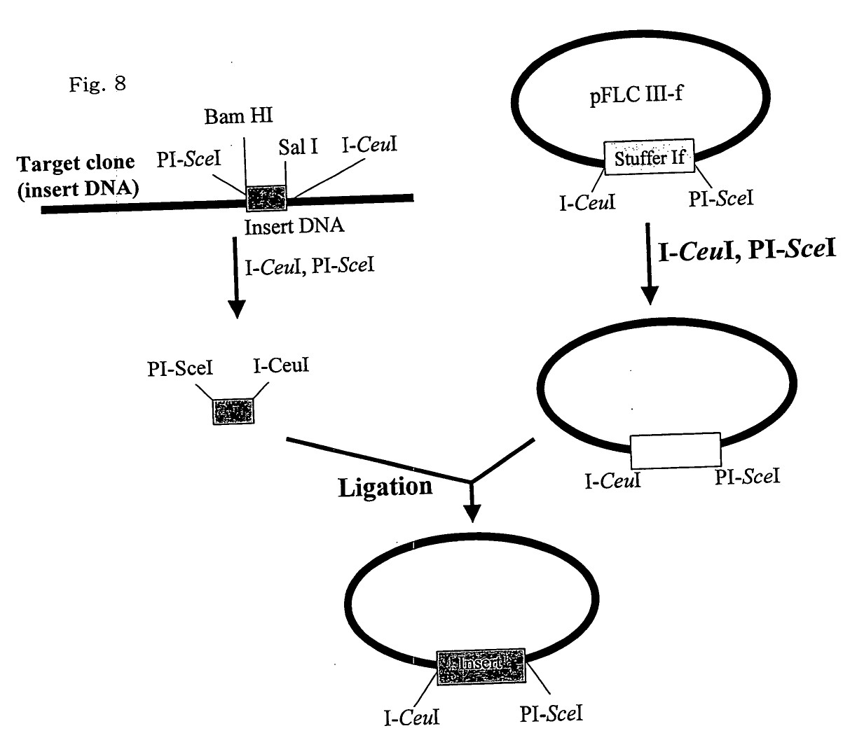

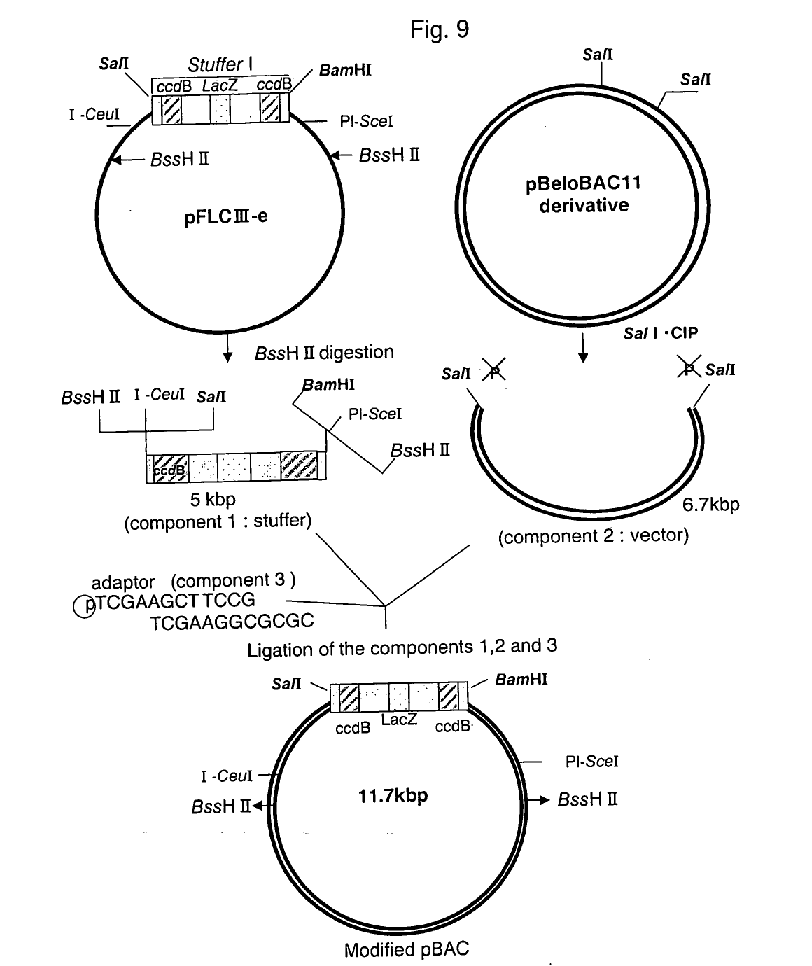

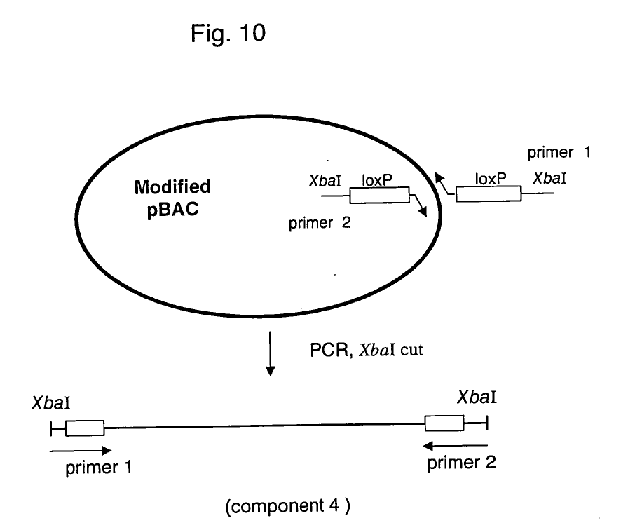

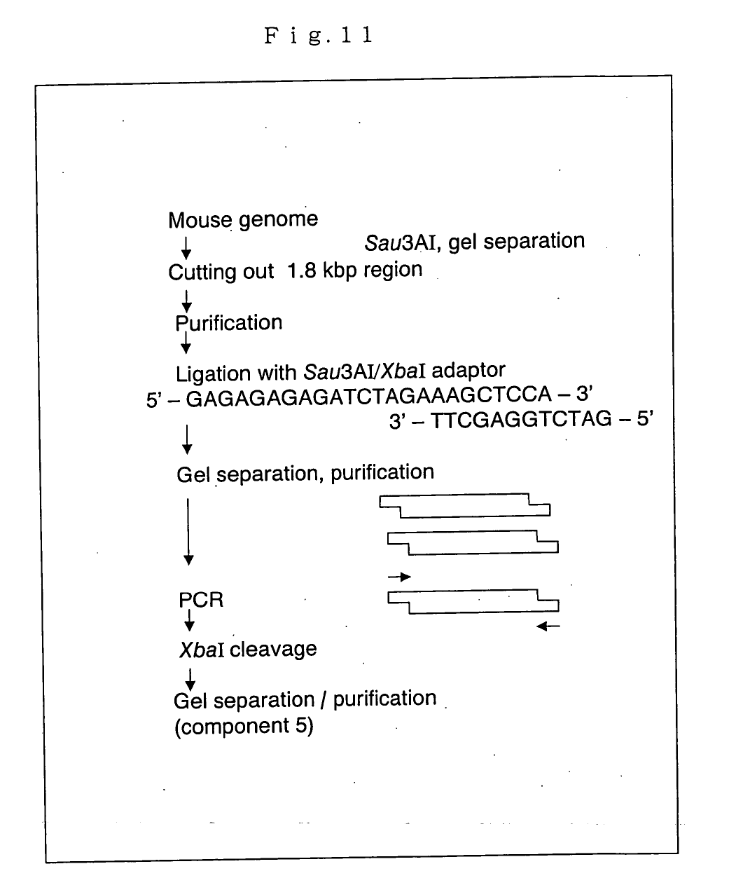

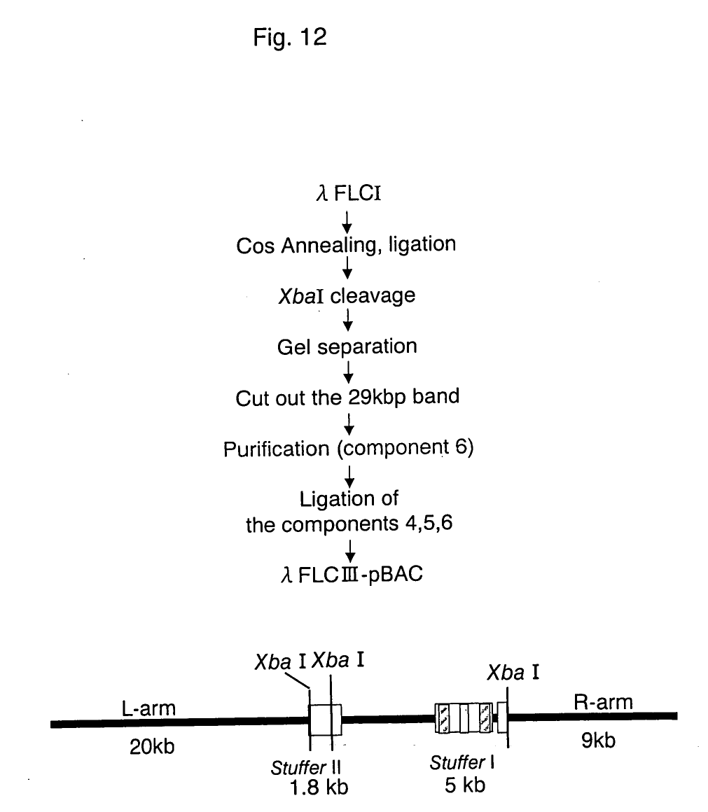

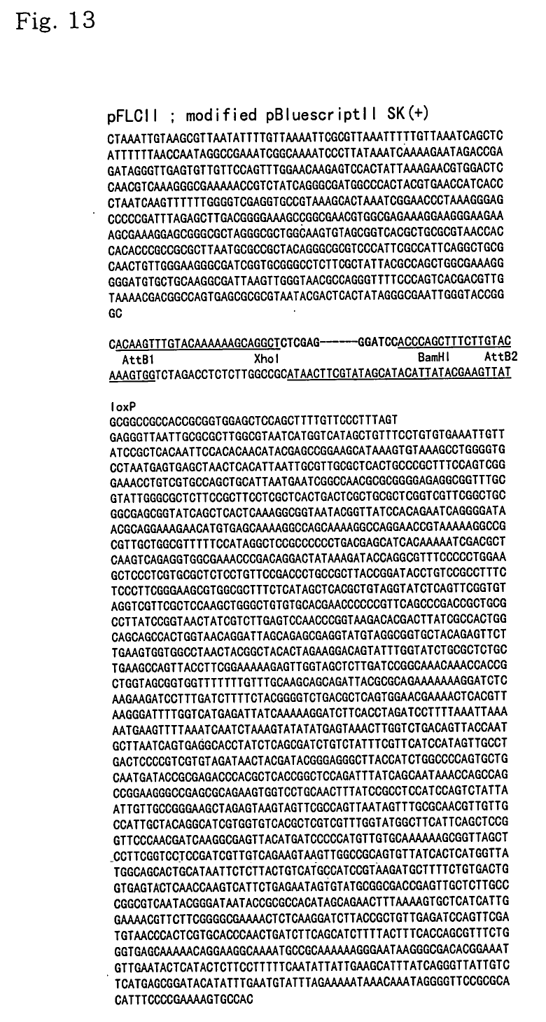

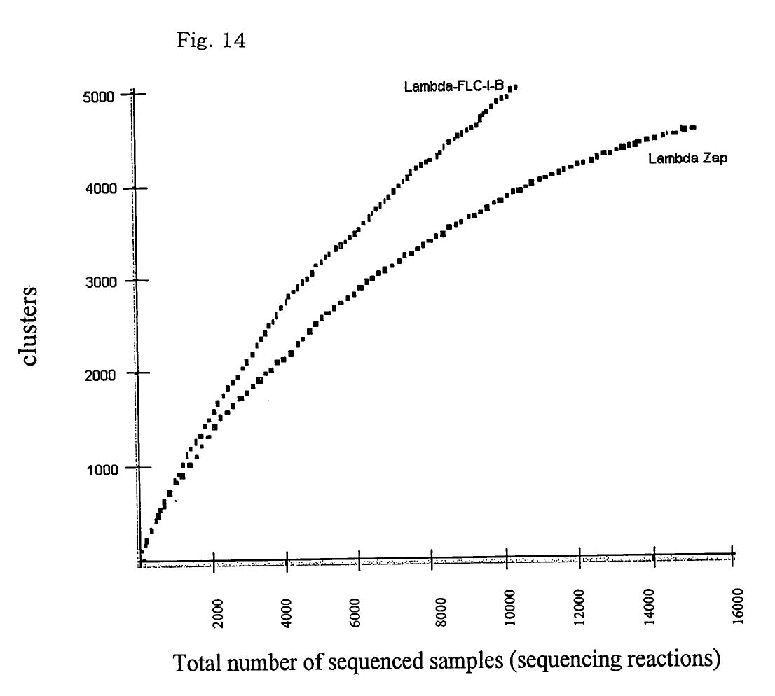

At the right side of FIG. 1(a), it is shown the mechanism of plasmid excision according to the cre-lox system or the excision of cDNA inserts into a destination or receiving vector with the Gateway™ system.

In FIGS. 1(b)-(f) various constructions and sizes of the stuffer I (RS) are shown: stuffer I of (b) is 10 Kb as from λ-PS vector; (c) is a short version of the stuffer I to simplify the arms purification; (d) is a 10 Kb stuffer with 4 ccdB and two LacZ to cut the background; (e) is a 5 Kb stuffer with 2 ccdB and one Lac Z; (f) is a stuffer for the ccdB and lox P double background cutting.

In particular, in (g), it is shown a non recombinant plasmid comprising the ccdB gene which inhibits growth, while LacZ (h) allows color selection. In (i) it is shown the background-reducing system using a loxP site, which separates the origin of replication and the resistant gene. Abbreviations: Sw=SwaI, Sf=SfiI, Sp=SpeI, Fs=FseI, Pa=PacI, Xa=XbaI.

The PacI, FseI, SfiI, SwaI, and the cloning sites cut only the sites that are shown and do not cut elsewhere in the vectors.

FIG. 2. Several constructions for vectors according to the invention, which are for simplicity indicated with the generic name of λ-FLC are shown.

(a) λ-FLC-I-B and λ-FLC-I-E, having the stuffer I of FIGS. 1b and 1e, respectively. (b) λ-FLC-I-L-B and A-FLC-I-L-D, which lack the stuffer II and have a stuffer I of FIGS. 1b and 1d, respectively, cloning site as in (a). (c) λ-FLC-II-C carrying the Gateway™ attB1 and attB2 sequence for bulk transfer of clones; it has a stuffer I like FIG. 1c. (d) λ-FLC-III-F having the stuffer I like in FIG. 1f for background reduction. (e) λ-FLC-III-L-D which lack the stuffer II and has the stuffer I like in FIG. 1d. (f) λ-FLC-III-S-F, having the stuffer I like in FIG. 1f but having a longer stuffer II (6.3 Kb).

Vectors (d-e) have sites for homing endonucleases (I-Ceul and PI-SceI) next to the cloning site for easy transfer of inserts to other vectors; the cloning site is shown in (d) only.

Vectors (g-j) show polylinker sequences which are placed at left and right side flanking the stuffer I (indicated in FIGS. 1(b-f)) or cDNAs (which is represented by a sequence of asterisks). The underlined sequences into the polylinkers represent primers, recombination sites, restriction sites, and the like. These restriction sites do not cut elsewhere in the λ-vectors or in the plasmids at all. More specifically, in pFLC-I, the left polylinker (SEQ ID NO:1) comprises: Forward (Fwd) M13 primer site, site for T7 polymerase, recombination site loxP, restriction sites SfiI and SalI site sequences; the right polylinkers (SEQ ID NO:2) comprises: restriction sites BamHI and SfiI, site for T3 polymerase, Reverse (Rev) M13 primer site. In pFLC-II, the left polylinker (SEQ ID NO:3) comprises: Fwd M13 primer site, T7, attB1, XhoI and SalI; the right polylinker (SEQ ID NO:4) comprises: BamHI, attB2, loxP, T3, Rev M13 primer site. In pFLC-III, the left polylinker (SEQ ID NO:5) comprises: Fwd M13 primer site, T3, I-CeuI, SalI; the right polylinker (SEQ ID NO:6) comprises: BamHI, PI-Sce T7, Rev M13 primer site. In pFLC-DEST, the left polylinker (SEQ ID NO:7) comprises: Fwd M13 primer site, T3, attB1, XhoI, SalI; the right polylinker (SEQ ID NO:8) comprises: BamHI, attB2, T7, Rev M13 primer site.

The general pFLC-II of FIG. 2h (i.e. without mentioning the specific stuffer I or the “insert cDNA”) can be constructed by using a modified pBluescriptII SK. A general pFLC-II having this construct is shown in FIG. 13 and the entire sequence (without stuffer I or “insert cDNA”) is shown in SEQ ID NO:51.

FIG. 3. Excision protocols. From left to right, in vivo solid phase Cre-recombinase (state of the art), in vivo liquid phase Cre-recombinase, in vitro Cre recombinase. On the right side, the “direct”, “indirect”, and “amplified indirect” protocols, which are mediated by the Gateway™ (GW) sequences and enzymes for in vitro excision.

FIG. 4. Average size of obtained cDNA libraries prepared with λ-Zap II or λ-FLC-I-B.

FIG. 5. This Figure shows possible vector constructions according to the present invention.

The vector according to the invention can be circular or linear, comprising a first segment indicated as construction segment (CS) and a second segment indicated as replaceable segment (RS). In linear form the construction segment (CS) of the vector is represented comprising a left segment and a right segment. RS is the segment which will be replaced by the nucleic acid insert of interest, for example a full-length cDNA.

The vector according to the invention can be circular or linear.

In (a) and (b) recombination sites (here generally indicated as att1 and att2), which do not recombine with each other, flanking RS, according to the Gateway™ recombination/excision system (Gateway™ Cloning Technology Manual, GIBCOBRL®, Life Technologies®) are shown.

In c) and d), recombination sites (lox site in this case), which recombine with each other by the Cre-lox recombination mechanism are present in CS.

In e) and f) it is shown that the Gateway-like sites flanking a RS and the recombination sites like the lox sites (shown in c) and d)) can be present at the same time.

In (g), recombination sites flanking RS are two lox sites, which do not recombine with each other. They work in the same way as the Gateway sites do.

In (h), it is shown the presence into RS of the gene ccdB as background-reduction.

In (i), it is shown the presence of a “third” lox recombination site as background-reducing sequence, capable of recombination with the lox site sequences in CS.

FIG. 6. Mechanism of action of a cloning vector comprising two homing endonuclease asymmetric recognition site sequences (a). These two sequences not capable of ligating with each other, are placed flanking a RS during the ligation process. Each of these sequences recognizes and ligates to one sequence flanking a nucleic acid insert of interest (b). Only ligation vector-insert is allowed. Ligations insert-insert or vector-vector are in this way avoided.

FIG. 7. It is described an example of preparation of λ-FLC-III-F. The stuffer If, is the stuffer I of FIG. 1f.

FIG. 8. It is disclosed an example of excision of asymmetric recognition site sequences, in the specific example using homing endonuclease I-CeuI and PI-SceI.

FIG. 9. It is described the preparation of a modified pBAC for the preparation of a λ-BAC vector. A detailed explanation of the process is disclosed in Example 20.

FIG. 10. It is described the insertion of loxP and XbaI sites into the modified pBAC of FIG. 7. A detailed explanation of the process is disclosed in Example 20.

FIG. 11. It is described a chart comprising the steps for the preparation of the stuffer II (“component 5”). A detailed explanation of the process is disclosed in Example 20.

FIG. 12. It is described a chart comprising the steps for the preparation of the λ-FLC-III-pBAC. A detailed explanation of the process is disclosed in Example 20.

FIG. 13. It is reported the full nucleotide sequence of an example of a general pFLC-II as described in FIG. 2h (that is, without showing the sequence of the stuffer I or the “insert cDNA”). The “insert cDNA” or stuffer I (indicated in FIG. 2h with a line of asterisks) is indicated in FIG. 13 by a line between the sequences CTCGAG - - - GGATCC. This construct of a general pFLC-II is a modified pBluescriptII SK(+).

The sequence of the plasmid of FIG. 13 is indicated in SEQ ID NO:51 as a single sequence starting from the sequence GGATCC (above), and terminating with the sequence CTCGAG (above), therefore without indicating the sequence of specific stuffer I or cloning cDNA.

FIG. 14. This graph compares cloning vector λ-FCL-I-B of the present invention and conventional ZAP vector in terms of cloning efficiency.

### DETAILED DESCRIPTION OF THE INVENTION

Full-length cloning has been hampered by problems related to both the preparation and cloning of long cDNAs. A consistent part of the problems has been overcome with the preparation of long cDNAs with thermostabilized and thermoactivated reverse transcriptase (Carninci et al., 1998, Proc. Natl. Acad. Sci. USA. 95: 520-524) and the development of cap-based full-length cDNA selecting techniques (Carninci et al., 1996, Genomics, 37: 327-336; Carninci et al., 1997, DNA Res., 4: 61-66; Carninci et al., 1999, Methods Enzymol., 303: 19-44; Carninci et al., 2000, Genome Res., 10: 1617-1630).

However, cloning methods and methods for preparing bulk cDNA libraries still showed a bias for short size cDNAs.

The present inventors provide a new family of vectors capable of cloning nucleic acids with wide range size and preferably very long and full-length cDNAs, high efficiency of excision and reduced background and contamination. Also provided are methods of cloning using such vectors.

According to a first embodiment, the invention provides a cloning vector comprising a construction vector segment (CS) and a replaceable segment (RS) (also indicated as “stuffer I”) (FIG. 1). RS is the segment that will be replaced by the nucleic acid insert of interest, which one intends to clone.

The bacteriophage or plasmid vector of the invention can be both linear or circular (FIG. 5,a-i). In case of a linear vector, the segment CS can be graphically considered as divided into two arms or segments, one at left side and the other at right side of RS. However, for more clarity the terminology of left arm or segment and right arm or segment of CS will be also maintained in case of circular vector.

The vector available in the state of the art was a modified λ PS vector having a “basic” size of 32 kb plus a 6 kb nucleic acid sequence (stuffer II), so that the size of the vector, without considering the cDNA of interest, was 38 kb (Japanese patent application having publication number P2000-325080A filed by the same applicant of the present invention). However, this vector had the disadvantage of bias for short and non full-length cDNAs, the presence of which are inconvenient for the preparation of a full-length cDNA library or encyclopedia.

The present inventors have surprisingly found that a vector, preferably a bacteriophage, more preferably a λ bacteriophage, having the size of CS of: 36.5 kb≦CS<38 kb, preferably CS is 37.5 kb, allowed the selection of long and full-length cDNA avoiding the problem of the λ phage of 38 kb.

The preferred size of 37.5 kb of CS according to the vector of the present invention is 0.2 kb shorter than the minimum size necessary for a λ-phage to undergoing packaging, which corresponds to 37.7 kb (Zabarovski et al., 1993, as above).

The advantages of the vector of CS 37.5 kb according to the invention compared to that of the state of the art of CS 38 kb is showed in Table 1.

The system for avoiding the bias for short and for the preferable preparation of full-length cDNAs can also be applied for bacteriophages different from λ.

Accordingly, the invention also relates to a cloning bacteriophage vector comprising a construction segment (CS) and a replaceable segment (RS), wherein the size of CS is: : X−1.2 kb≦CS<X; X (expressed in kb) corresponding to the minimum size necessary to the bacteriophage vector for undergoing packaging (which nominally is 37.7 kb for λ, as reported in Zabarowski et al., as above). The size of CS is preferably: X−0.2 kb.

The diminution of a short fragment from the size of X renders the CS fragment below the packaging level, however, the presence of the RS (also indicated as “stuffer I”) makes the bacteriophage vector capable of packaging.

In FIGS. 1 and 2, the vector according to the invention is constructed inserting a stuffer II of the desired size. Preferably, of 5.5 kb, so that the CS corresponds to a size of 37.5 kb. However, the stuffer II can be: 4.5≦stuffer II<6. The stuffer II can be of any origin and any nucleic acid. It can be a foreign sequence fragment, for example a mouse genomic DNA or can be taken from plasmid. The stuffer II can also be already originally present in the vector.

The CS of the vector according to the invention can preferably be a bacteriophage segment, or comprise a bacteriophage fragment. Preferably, the bacteriophage is a λ bacteriophage. A list of available bacteriophage and λ bacteriophage has been reported in the state of the art of the present application (see for example those reported in Sambrook et al., 2.16-2.53) or derivatives thereof.

CS can also be modified by comprising a plasmid segment at least comprising a ori. The plasmid comprising ori is preferably selected from the group of: pBluescript (+), pUC, pBR322, and pBAC. In FIG. 1, for example, a fragment of a modified pBluescript(+) comprising ori has been inserted into the left arm of CS. An example of use of pBAC or derivative thereof for the preparation of vectors according to the invention is given, for example in FIGS. 9-12 and Example 20. However, pBAC or its derivative can be efficiently used for the preparation of any vector contruct according to the invention. Examples of vectors and linker, adapter, primer sequences and the like that can be used in the construction of the vectors according to the invention are reported in the NCBI VecScreen, UNIVEC Build #3.2 Database (National Centre for Biotechnology Information, National Library of Medicine, National Institute of Health, US). Specific information about these vectors can also be found in the Catalog of Amersham Pharmacia Biotech, Inc., US; Clontech Laboratories, Inc, US; Invitrogen Corporation, US; Life Technologies, Inc., US; New England Biolabs, Inc., US; Promega Corporation, US; and Stratagene, US.

The cloning vector according to the invention can also comprise a selectable marker. Accordingly, CS comprises at least a selectable marker selected from the group consisting of: a DNA segment that encodes a product that provides resistance against otherwise toxic compounds (e.g. antibiotic resistant gene); a DNA segment that encodes a product that suppresses the activity of a gene product; a DNA segment that encodes a product that is identifiable (e.g. phenotypic markers such as beta-galactosidase, green fluorescent protein,(GFP), and cell surface proteins); a DNA segment that encodes a product that inhibits a cell function; a DNA segment that provides for the isolation of a desired molecule (e.g. specific protein binding sites); and a DNA segment that encodes a specific nucleotide recognition sequence which is recognized by an enzyme.

The selectable marker is more specifically at least a marker selected from the group consisting of an antibiotic resistance gene, an auxotrophic marker, a toxic gene, a phenotypic marker, an antisense oligonucleotide; an enzyme cleavage site, a protein binding site; and a sequence complementary to a PCR primer sequence.

Amp as an example of selectable marker is showed in FIGS. 1 and 2.

The RS of the vectors of the invention can be flanked by two recombination sites (as showed in FIGS. 1, 5) wherein these two recombination sites do not recombine with each other. More in particular, these recombination sites are selected from the group consisting of attB, attP, attL, and attR or their derivatives for carrying out the recombination excision according to the Gateway™ methodology (Walhout et al., 2000, as above; Life Technologies catalogue; Gateway Cloning Technologies, Instruction Manual, GibcoBRL, Life Technologies; and U.S. Pat. No. 5,888,732). The complete list of Gateway recombination sites and derivatives is disclosed in the above Life Technologies references.

The Gateway™ system has been proposed in the art for exchange of components between plasmids and for transferring a nucleic acid insert of interest into a specific functional plasmid. However, the Gateway system showed a bias for short cDNA; long cDNAs are obtained with low efficiency (Michael A. Brasch, slide “Gateway cloning of attB-PCR products”, GIBCOBRL® Technical Seminar, “Gateway Cloning Technology”, Life Technologies™, 1999).

The present inventors have instead surprisingly found that when Gateway recombination sites are transferred into a bacteriophage vector according to the present invention and positioned flanking the RS (as shown in FIGS. 1, 2 and 5,a,b,e,f) the cloned cDNA library did not show bias for short cDNAs.

The present invention therefore, provides a bacteriophage vector, preferably having a CS size of: 32 kb≦CS<45 kb, in particular 36.5 kb≦CS<38 kb, more preferably CS is 37.5 kb comprising two recombination sites, which do not recombine with each other, flanking RS (FIG. 5,a-g). The bacteriophage is preferably a λ bacteriophage.

The bacteriophage vector according to the present invention, however, is not limited to λ bacteriophage but other bacteriophage known in he art can be used (for example those described in Zabarovski et al., 1993, as above).

In the vector according to the present invention, in alternative to the Gateway attB, P, L or R or their derivatives, two lox recombination sites flanking RS (for example, two generic lox1 and lox2 sites are shown in FIG. 5,g) can be used. These lox recombination sites can be any mutated or derived lox sites, for example a mutated or derived loxP site (for example loxP511) as described in Hoess et al., NucleicAcids Res., 1986, 14(5):2287.

The vector according to the invention can also comprise two lox recombinant sites each of them placed in each arm (or segment portion) of CS (FIGS. 1, 2, and 5,c-f,i), that is, one lox site placed in the CS, at the left side of the RS (or of the nucleic acid of interest) and the other lox site in the CS, at the right side of the RS (or of the nucleic acid insert of interest); these lox recombination sites being capable to recombine with each other.

These sites can be two lox recombination sites modified, mutated or derived lox site (Hoess et al., 1986, as above), preferably a loxP or a modification or derivative thereof For example, the lox sites can be loxP 511 (Hoess et al, 1986, as above). A loxP 511 recombines with another loxP 511 site, but not with a loxP site. All the above variation, mutation, modification or derivation of lox site, will be generally indicate as “lox site and derivative thereof”, for the purpose of the present application.

In this case, after the RS is substituted by the nucleic acid insert of interest, the recombination is carried out by a Cre-lox recombinase.

The Cre-lox recombination system is described in several prior art references, for example, Palazzuolo et al., 1990, as above; Elledge et al., 1991, as above; and Summers et al., 1984, as above.

In alternative, to the Cre-lox recombinase system, other recombination systems can be used for the purpose of the present invention. Among them, Kw recombinase (Ringrose L., et al., 1997, FEBS, Eur. J Biochem., 248:903-912), hybrid site-specific recombination system with elements from Tn3 res/resolvase (Kilbride E., et al., 1999, J. Mol. Biol., 289:1219-1230), β recombinase system (Canosa I., et al., 1998, Journal Biological Chemistry, Vol.273, No.22, May 29:13886-13891); FLP recombinase system (Huffman K. E., and Levene S. D., 1999, J. Mol. Biol.,:286:1-13; and Waite L. L., and Cox M. M., 1995, Journal Biological Chemistry, Vol.270, N.40:23409-23414). Modification, mutation or derivative of these recombination sites can also be used and they will be generally indicated as “derivative thereof”.

The result of this recombination process, mediated by Cre-recombinase or other recombinases, is the excision of a plasmid comprising the nucleic acid of interest.

According to an embodiment of the invention, the presence of both the recombination sites flanking RS for the recombination Gateway-like system and the recombination sites in the two arms of CS for Cre-lox, Kw, Tn3 res/resolvase, β recombinase, and FLP recombination, into a vector, renders said vector particularly suitable for cloning, transfer of nucleic acid material of interest, and preparation of libraries. In fact, according to the particular case, the most convenient excision system can be chosen without changing or modifying the vector.

According to a further aspect, the cloning vector according to the invention can also be used for cloning or for preparing libraries with low or no background. Accordingly, the present invention provides a cloning vector comprising a construction segment (CS) and a replaceable segment (RS), wherein said CS is a bacteriophage vector segment and said RS comprises at least the ccdB gene as background-reducing system.

The bacteriophage or plasmid cloning vector according to the invention, can also comprises a construction segment (CS) and a replaceable segment (RS), wherein said CS is a bacteriophage or a plasmid vector segment and i) said RS comprises at least a recombination site (capable of recombination with the two recombination sites present in the left and right arms of CS) as background-reducing system, or ii) RS is flanked by two endonuclease asymmetric recognition site sequences which do not ligate with each other and are recognized by restriction endonucleases.

The recombination site comprised into RS must be able to recombine with the recombination sites present into the left and right arms of CS, therefore, we can address to this RS recombination site as the “third” recombination site.

The “third” recombination site can be a lox recombination site or a derivative thereof, preferably a loxP site or derivative thereof.

The two endonucleases asymmetric site sequence background-reducing systems can be for example: i) homing endonuclease asymmetric recognition site sequences, or ii) asymmetric restriction endonuclease cleavage site sequences recognizable by class IIS restriction enzymes.

The background-reducing bacteriophage vector has preferably the size of CS: 32 kb≦CS≦45 kb, advantageously CS is: 36.5 kb≦CS<38 kb, more preferably CS is 37.5 kb. The bacteriophage is preferably a λ bacteriophage.

The bacteriophage CS or the vector can comprise a plasmid segment at least comprising an ori. The plasmid segment comprising an ori is preferably, but not limited to, selected from the group consisting of: pBluescript(+), pUC, pBR322 and pBAC, or any plasmid as included into the NCBI Database, as above.

In case of the background-reducing plasmid, this can be any kind of plasmid known in the art, for example any of the plasmid above indicated or disclosed in the NCBI Database.

This vector preferably comprises at least a selectable marker selected from the group as above disclosed. In particular, the at least selectable marker can be selected from the group consisting of an antibiotic resistance gene, an auxotrophic marker, a toxic gene, a phenotypic marker, an enzyme cleavage site, a protein binding site; and a sequence complementary to a PCR primer sequence.

The background-reducing cloning bacteriophage or plasmid vector can also comprise at least one of the recombination system as above described, that is i) two recombination sites which do not recombine with each other flanking RS (Gateway sites or lox modified sites) and/or ii) at least two recombination sites which recombine with each other placed into the two arms of CS, recognized by a recombinase. These recombination sites capable of recombining with each other, are preferably selected from the group consisting of: lox sites, Kw, Tn3 res/resolvase, β recombinase sites, and FLP sites, as described above.

With reference to the background-reducing ccdB system, it has been disclosed into plamids by Bernard P. and Couturier M. (1992, J. Mol. Biol., 226:735-746) and also Walhout et al. (as above) for the Gateway™ vectors.

The product of the ccdB gene interferes with DNA gyrase. After recombination, only the plasmids that have lost the ccdB gene (and which are recombinant) can grow in E.coli strains not mutated for gyrA, therefore providing a selective advantage (see Life Technologies references).

Plasmids carrying the gene ccdB can propagate only in specific E.coli strains. For example in DB3.1, which carries a mutation in gyrA gene conferring resistance to ccdB (Walhout et al., as above). Therefore, this kind of recombination is limited to plasmids, because bacteriophage vectors, for instance λ substitution vectors, used in cloning systems cannot grow and replicate in cells like DB3. 1, which lack the recA protein (the recA product is required for the growth of substitution-type bacteriophage λ: Sambrook et al., 1989).

The present inventors have instead surprisingly found that a bacteriophage, preferably a λ bacteriophage, comprising at least a ccdB gene into the RS, according to the invention can propagate and multiply on a culture of C600cells. On the contrary, plasmids comprising the ccdB gene cannot propagate in C600 cells.

The mechanism of the background-reducing ccdB system in the vector of the invention is shown in FIG. 1,g.

During the replacement of the RS with the nucleic acid insert of interest, it may happen that no replacement occurs or an imperfect ligation or replacement is realized. In this case, bacteriophage or plasmid vectors without complete nucleic acid insert of interest are present in the culture creating background. With the presence of ccdB, the “suicide gene”, the background or byproduct can be reduced about or very closed to zero.

A problem of background contamination can also occur during the purification, when the removal of stuffer I (RS) is realized on gel (for example agarose gel) and fragment of stuffer I nucleic acid is collected with CS and can therefore be reinserted into the vectors.

Another background-reducing system is the “third” recombination site, which is placed into RS and is capable to recombine with the recombination sites present into the left and right arms of CS of the bacteriophage or plasmid vector of the invention (FIG. 1,i; FIG. 5,i). This “third” recombination site can be in presence or in absence of the ccdB gene.

Preferably, this background-reducing “third” recombination site is a lox site or a derivative thereof, more preferable a loxP site or a derivative, modification or mutation thereof, as above described. However, the background recombination site present into RS, must be capable of recombination with the two recombination sites present in the two arms of CS. Therefore, in case of recombination mediated by Cre-recombinase, all the three sites have to be lox-recombination or derivatives thereof, capable of recombining with each other.

For example, in FIGS. 1,a and 1,f, the two recombination sites present in the left and right arms of CS (of a bacteriophage or a plasmid vector) and the background-reducing “third” recombination site into RS (stuffer I) are all loxP sites.

In FIG. 1.i), it is explained the mechanism of action of the “third” recombination site. In case of imperfect ligation of the nucleic acid insert of interest, one of the loxP site in arms of CS preferably recombine with the “third” loxP forming, during the excision step, an excised plasmid, which in one case lack the ori and cannot replicate, and in the other case lack the selectable marker (Amp in the Figure) and cannot grow up.

Accordingly, the present invention also relates to a method for cloning or preparing bulk library with low or no background using a bacteriophage or plasmid vector comprising at least the “third” recombination site as described.

The background-reducing “third” recombination site can be any recombination site other than lox, for example the recombination sites used for the recombination as above described.

The background-reducing bacteriophage or plasmid cloning vector according to the invention, can also comprises the lacZ gene into RS even in presence of the ccdB gene or the “third” recombination site or the like, or in presence.

The bacteriophage or plasmid cloning vector according to the invention, in alternative or in presence of the background-reducing sequences above described, can also comprise two asymmetric sites recognized by restriction endonucleases. These two asymmetric site sequences flank the RS of the vector (FIG. 6).

Asymmetric site sequences useful for the purpose of the present invention are: i) two homing endonuclease asymmetric recognition site sequences or ii) restriction endonuclease asymmetric cleavage sites sequences recognizable by class IIS restriction enzymes.

Homing endonucleases are sold and described by New England Biolabs, Inc. A; a description of the asymmetric site sequences is also available in the New England Biolabs Catalog. These homing endonuclease asymmetric recognition site sequences are from 18 to 39 bp. However, in the present invention the recognition site sequences are not limited to those sequences nor to these sizes. The New England Biolabs Catalog reports that after 5-fold overdigestion with I-Ceu-I, greater than 95% of the DNA fragments can be ligated and recut with this enzyme.

Preferably, the restriction homing endonucleases capable of cutting the asymmetric site sequences are selected from the group consisting of: I-CeuI, PI-SceI, PI-PspI and I-SceI.

FIG. 6,a) shows a vector being removed of its RS, bringing two homing endonoclease recognition site sequences, which do not ligate with each other, at the extremities of the CS arms; the RS being removed by using the homing endonucleases specific for those site sequences. In FIG. 6,b) a nucleic acid insert of interest having a pair of homing endonuclease site sequences placed flanking said insert of interest (these sequences being the same of those of the vector) is provided for the ligation to a vector having RS removed. In FIG. 6,c) one homing endonuclease site sequence of the vector recognizes and hybridizes to a complementary homing endonuclease site sequence of the insert. In FIG. 6,d), the second homing endonuclease site sequence of the vector, after a certain time, preferably overnight, recognizes and hybridizes the complementary homing endonuclease site sequence placed on the other extremity of the insert of interest. In conclusion, using this system, after a certain time, all the complementary site sequences of the inserts recognizes and hybridize with their complementary site sequences of the vectors. As consequence, insert-vector ligation is carried out. Both insert-insert and vector-vector ligations are not realized since they extremities are not complementary reducing by-products. With this system, also nucleic acid contamination entering the vector is reduced.

The homing endonuclease recognition site sequences can also be placed into a destination vector, preferably a plasmid, and the subcloning process can be advantageously carried out. This vector ligates with the nucleic acid insert of interest, which brings two endonuclease recognition site sequences, which are the same of the destination vector, placed flanking this nucleic acid insert of interest.

The same process can be realized when asymmetric site sequences recognized by class IIS endonuclease enzymes are used instead of the homing endonuclease site sequences. Examples of class IIS restriction enzymes include, AlwI, AlwXI, Alw26I, BbsI, BbvI, BbvII, BcsfI, BccI, BcgI, BciVI, BinI, BmrI, BpmI, BsaI, BseRI, BsgI, BsmAI, BsmBI, BspMI, BsrDI, BstF5I, EarI, Eco31I, Eco57I, Esp3I, FauI, FokI, GsuI, HgaI, HinGUII, HphI, Ksp632I, MboII, MmeI, MnlI, NgoVIII, PleI, RlaAI, SapI, SfaNI, TaqII, TthlllII, BsnIs, BsrIs, BsmFI, BseMII, and the like (see Szybalski W., et al., 1991, Gene, 100, 13-26; and Catalog of New England Biolabs, Inc.).

Examples of recognition sites and cleavage sites of several restriction enzymes are (into parenthesis are the recognition site and the cleavage site):

| | |
|---|---|
| BbvI | (GCAGC 8/12), |
| HgaI | (GACGC 5/10), |
| BsmFI | (GGGAC 10/14) |
| SfaNI | (GCATC 5/9), |
| and | |
| Bsp I | (ACCTGC 4/8). |

The endonuclease asymmetric recognition site sequences as described above can be placed into the bacteriophage or plasmid cloning vector according to the invention also in presence of, the ccdB gene, the lacZ gene, and/or the “third” background-reducing recombination site (for example lox) into RS.

The vector ligated with the endonuclease asymmetric system as described above can then be excised by any of the recombination system present in CS, as above described, for example cre-lox recombinase, preferably loxP, Kw, FLP, Tn3 res/resolvase, β recombinase, etc. The vector comprising the endonuclease asymmetric according to the invention, therefore, also comprises at least a pair of recombination sites into the CS.

The RS (or stuffer I) of the cloning vector according to the invention is removed by the vector and it is replaced by the nucleic acid insert of interest with the ligation process.

The nucleic acid insert of interest which is used in all of the embodiments of the present application is selected from the group consisting of DNA, cDNA, RNA/DNA hybrid. Advantageously, long cDNA and preferably full-length cDNA. The full-length cDNA is preferably a normalized and/or subtracted full-length cDNA.

Any of the vectors according to the invention has proven to be particularly useful for cloning nucleic acids of interest and for the preparation of library, in particular full-length cDNA library/libraries. Accordingly, the present invention relates to a method for cloning at least a nucleic acid insert of interest or for preparing at least a bulk nucleic acid library of interest, comprising the steps of:
a) preparing at least a cloning vector according to the invention;
b) replacing RS with a nucleic acid insert of interest into the cloning vector obtaining a vector comprising the nucleic acid insert of interest;
c) allowing the in vivo or in vitro excision of the nucleic acid insert of interest or of the plasmid comprising the nucleic acid insert of interest;
d) recovering the (recombinant) plasmid carrying the nucleic acid insert of interest or the library of (recombinant) plasmids carrying the nucleic acid inserts of interest.

Optionally, between step b) and c), a step of amplification of cloning vector can be carried out.

The method according to the invention can also be used for cloning nucleic acid insert of interest or for preparing a bulk nucleic acid library of interest with reduced or no background.

Accordingly, the present invention provides a method for cloning a nucleic acid insert of interest or for preparing a bulk nucleic acid library of interest, with low or no background, comprising the steps of:
(a) preparing at least a cloning vector according to the invention comprising a background-reducing system as above described;
(b) replacing RS of vector of step (a) with a nucleic acid insert of interest;
(c) allowing the in vivo or in vitro excision of the nucleic acid insert of interest or of the plasmid comprising the nucleic acid insert of interest;
(d) recovering the (recombinant) plasmid carrying the nucleic acid insert of interest and lacking of the background-reducing sequence or a library of said plasmids.

Optionally, an amplification step is carried out between the steps b) and c).

The background-reducing system according to the invention can be the gene ccdB or a “third” recombination site sequence (capable of recombination with the two lox recombination sites present into the left and right arm of CS), which is placed into the RS of the bacteriophage or plasmid vector according to the invention. The “third” recombination site is preferable a lox site or derivatives thereof, more preferably a loxP site or derivatives thereof.

In case of a Gateway-like method, the gene ccdB is instead placed into the RS of a destination vector.

The bacteriophage or plasmid vector or the destination vector can also comprise the lacZ gene.

In Alternative, in the background-reducing method according to the invention, the bacteriophage or plasmid vector can comprise two endonuclease asymmetric recognition site sequences flanking RS.

Accordingly, the present invention also relates to a method for cloning a nucleic acid insert of interest or for preparing a bulk nucleic acid library of interest, comprising the steps of:
(a) preparing at least a bacteriophage or plasmid vector comprising two endonuclease asymmetric recognition site sequences placed flanking RS of said vector;
(b) replacing RS with a nucleic acid insert of interest comprising two endonuclease asymmetric recognition site sequences flanking said insert of interest, said sequences being capable of ligating with the two sequences placed into the vector of step a), and obtaining a vector comprising the nucleic acid insert of interest;
(c) allowing the in vivo or in vitro excision of the nucleic acid insert(s) of interest or of at least a plasmid comprising the nucleic acid insert of interest;
(d) recovering the (recombinant) excised plasmid or destination plasmid carrying the nucleic acid of interest or a library of said plasmid(s) with low or no background.

Further, the present invention relates to in vivo and in vitro Cre-lox recombination system, using the vector according to the invention.

As discussed in the state of the art section, the Cre-recombinase solid-phase in vivo excision (see also FIG. 3 of the present application) known in the art (Palazzolo et al., 1990, Gene, 88:25-36) shows drawbacks as low plasmid yield (Palazzolo et al., 1990, as above) and plasmid instability; in fact Cre-recombinase is constitutively expressed causing formation of plasmid dimmers/multimers leading to high proportion of plasmid-free cells, impairing the sequencing efficiency (Summers et al., 1984, Cell, 36:1097-1103).

A Cre-recombinase liquid-phase in vivo excision, however, has not been successufully used in the state of the art because in liquid culture, cells comprising short plasmids replicate faster than cells comprising very long plasmids creating a bias for short plasmids (that is short nucleic acid insert of interest), and serious difficulty in obtaining long or full-length nucleic acid inserts.

The present inventors have surprisingly found that the drawbacks of the state of the art could be avoided essentially by allowing an excision of plasmids in liquid-phase under condition of very low or no growth (replication) and amplification, extraction of nucleic acid inserts of interest, preparation of different plasmids capable to growth in cells do not expressing Cre-recombinase, and further growth (amplification) in solid phase (on plate).

Accordingly, the present invention provides a method for cloning at least a nucleic acid insert of interest or preparing at least a bulk nucleic acids library of interest comprising the steps of:
a) preparing at least a cloning vector, comprising a construction segment (CS) and a replaceable segment (RS), wherein said CS is a bacteriophage vector comprising at least two lox recombination sites or derivatives thereof positioned in the left and right arm of CS.;
a) replacing RS with a nucleic acid insert of interest into the cloning vector;
b) packaging of the vector;
c) in vivo in liquid-phase infection of at least a cell expressing cre-recombinase;
d) allowing the in vivo in liquid-phase excision of a plasmid comprising the nucleic acid insert of interest under condition of short-time growth or no growth of the excised plasmid;
e) carrying out the cellular lysis and recovering the plasmid carrying out the insert or of a library of these plasmids.

This method, optionally comprises the steps of:
f) electroporating or transforming at least a cell, not expressing Cre-recombinase, making the plasmid(s) of step f) penetrating into said cell(s);
g) plating of cell(s) infected as at step g) and recovering the plasmid carrying the nucleic acid insert of interest or a library of said plasmids.

The electroporation is carried out according to the well-known mwthodology in the art. The transformation is preferally carried out by chemical treatment, for example, according to Sambrook et al., 1.71-1.84.

The bacteriophage vector according to this method is preferable a λ bacteriophage.

The lox recombination sites, which recombine with each other, can be any mutated, modified or derived lox site as above described, preferable a loxP, which can be mutated, modified or derived (therefore, generally indicated as loxP or derivatives thereof”).

The step e) of this method is preferably carried out in 0-3 hours at a temperature of 20-4° C. The temperature is preferably from room temperature to 37° C.

The present inventors have also developed a new and inventive in vitro Cre-lox recombination method.

In this in vitro method, a bacteriophage vector comprising the nucleic acid insert of interest is packaged in vitro in presence of (bacterial) packaging extract as known in the state of the art (for example, Gigapack® or Gigapack Gold® or the like, Stratagene, US). The nucleases present in the extract cut the short nucleic acids which have not been packaged and the nucleic acid contamination in general. The result is that the nucleic acid of the vector which has been packaged result purified.

In a preferred case, when a vector comprising the stuffer II of 5.5 kb (or a bacteriophage vector having the size of CS of 37.5 kb) is used, the short and not full-length cDNA having sizes below 0.5 kb are not packaged and are removed by the esonuclease. The result is a library with low or without bias for short cDNA. This library results to be very useful for the preparation of very long and full-length cDNAs.

Accordingly, the present invention provides a method for cloning at least a nucleic acid insert of interest or at least a bulk nucleic acid library of interest comprising the step of:
(a) preparing at least a cloning vector, comprising a construction segment (CS) and a replaceable segment (RS), wherein said CS is a bacteriophage vector segment comprising two lox recombination sites or derivatives thereof positioned in the left and right arm of CS;
(b) replacing RS with a nucleic acid insert of interest into the at least a cloning vector;
(c) in vitro packaging of the bacteriophage cloning vector of step b) in presence of packaging extract;
(d) extraction of bacteriophage cloning vector(s) from the capside;
(e) in vitro excision of the plasmid(s) comprising the nucleic acid insert(s) of interest from the vector in presence of Cre-recombinase;
(f) recovery of said plasmid or library of plasmids.

This method may further comprise the steps of:
(g) electroporating or transforming at lest a cell, not expressing Cre-recombinase, making said plasmid(s) entering into said cell(s);
(h) plating the cell(s) of step g) and recovering plasmid carrying the nucleic acid insert of interest or a library of said plasmids.

Optionally, between the steps c) and d) an amplification step on plate of the bacteriophage can be carried out.

The lox recombination sites can be lox sites mutated, modified or derivative thereof, preferably loxP or derivatives thereof.

The bacteriophage used in this in vitro Cre-lox method is preferably a λ bacteriophage.

Further, the present inventors have developed a method based on the Gateway mechanism from transferring nucleic acid insert of interest from the vector according to the invention into at least a destination functional vector. This functional vector can be utilized for different uses, for example for sequencing, for expressing a protein in bacteria or eukaryotic cells, making a protein fusion product, and so on.

The Gateway method as already said above is related only to plasmids and shows a strong bias for short cDNAs. In the Gateway method, cDNAs are amplified by PCR and inserted into the plasmid destination vector. However, the reaction times of PCR or full-length cDNAs are very long and generally the reaction is carried out overnight, which means low efficiency and size bias. Fragments with short insert recombine faster than fragment with long inserts. Therefore, when mixed, there is always size bias, the shortest competes with longer and the short is more efficiently cloned causing size bias.

The present inventors have solved this bias problem of the Gateway method.

The method according to the present invention comprises a step of ligating nucleic acids of interest (of different size) into the bacteriophage vector.

The bacteriophage vector according to the invention has bigger size (for example 37.5 kb plus the nucleic acid insert) than the donor vector of the Gateway method. A vector having the CS size according to the invention does not discriminate between short and long insert and vectors comprising both kid of inserts can be amplified and/or excised with a similar efficiency, so that there is no bias for short nucleic acid inserts.

Accordingly, the present invention provides a “Gateway-like” method for cloning at least a nucleic acid insert of interest or for preparing at least a bulk nucleic acid library of interest, comprising the steps of:
(a) preparing at least a cloning vector comprising a construction segment (CS) and a replaceable segment (RS), wherein said CS is a bacteriophage vector segment and RS is flanked by two recombination sites, wherein these recombinant sites do not recombine with each other;
(b) replacing said RS with a nucleic acid insert according to the invention;
(c) in vitro packaging the at least one bacteriophage cloning vector of step b);
(d) allowing the in vitro excision of the nucleic acid insert of interest by providing to the cloning vector of step c) at least a destination vector comprising a destination replaceable segment (RS) flanked by two recombination sites, which are capable of recombining with the recombination site of cloning vector(s) of step (a);
(e) recovering a recombinant plasmid carrying the nucleic acid insert of interest or a library of said plasmids.

Preferably, the bacteriophage is a λ bacteriophage.

The two recombination sites which do not recombine with each other flanking the RS of the bacteriophage cloning vector or of the destination vector, can be i) recombination sites selected from the group consisting of attB, attP, attL, and attR or derivatives thereof, or ii) lox recombination site or derivatives thereof, preferably loxP or derivative thereof (for example loxP and loxP511).

After the nucleic acid of interest has been transferred into the destination vector using the Gateway technology, said acid nucleic of interest can be transferred in a further destination or receiving vector according to the following procedures named as: i) GW direct; ii) GW indirect; and iii) GW amplification method, according to FIG. 3 and to the examples.

The excised plasmid or destination plasmid bringing the nucleic acid insert of interest according to the invention can be used as driver in a normalization and/or subtraction method.

A method for normalization and/or subtraction of a cDNA library, preferably a full-length cDNA library, has been disclosed by Carninci et al., 2000, Genome Res.,10:1617-1630.

Accordingly the present invention relates to a method for preparing at least a normalized and/or subtracted library comprising the steps of:
(a) providing at least a plasmid excised or a destination plasmid prepared according to the method of the present invention;
(b) providing the plasmid of step b) to a pool of nucleic acid targets;
(c) removing the plasmid/target hybrids;
(d) collecting the normalized and/or subtracted nucleic acid targets, which did not hybridize to the plasmid of the invention.

According to an embodiment, the plasmid of step a) is rendered as single strand. For example, it is treated by making at least a nick into one strand of the double stranded plasmid. Then, the strand which has been nicked is removed, finally steps (c)-(d) are applied.

Preferably, the nick is introduced by using the protein GeneII (Gene-trapper Kit, Gibco, Life Technologies, US) and the strand which has been nicked is removed by an exonuclease. The exonuclease is preferably ExoIII.

According to a further embodiment, the present invention relates to a method for preparing at least a normalized and/or subtracted library comprising the steps of:
(a) providing at least a vector according to the invention comprises a construction segment (CS) and a replaceable segment (RS), wherein CS comprises a F1 ori;
(b) replacing RS with a nucleic acid insert of interest according to the invention;
(c) adding an helper phage and producing a number of a single strand DNA (ssDNA) vector copies, secreted from the cells;
(d) providing the copies of step c) to a pool of nucleic acids targets;
(e) removing the plasmid/target hybrids;
(f) collected the normalized and/or subtracted nucleic acid targets, which did not hybridize with the target(s).

Helper phage is preferably obtainable from Stratagene. A more detailed description of a method for preparing ssDNA vector, consisting in infecting the bacterial cells with a helper phage (Stratagene catalog), then recovering the single strand plasmid secreted from the cell, extracting the DNA, and finally recovering the DNA from single strand plasmid can be found in the Stratagene User Manual of pBluescript. A method using the helper phage for reducing the vector at single strand is also described in (Bonaldo et al, 1996, Genome Res., 6:791-806).

When using the f1(+) origin of replication, an helper phages such as R408 can be used (Short et al., 1988, as above).

The bacteriophage vectors according to the invention can be prepared using any kind of plasmid or plasmid fragment known in the art, for instance pBluescript(+), pUC, pBR322, bacterial artificial chromosome plasmid (pBAC), pBeloBAC11 (Kim et al., 1996, Genomics, 34:213-218, a modified or derivative pBeloBAC11 according to U.S. Pat. No. 5,874,259 (herein incorporated by reference), or any other plasmid as listed public database or available from Company's Catalogues as above indicated.

Acording to one embodiment, the invention provides a bacteriophage vector comprising a bacterial artificial chromosome (pBAC) or pBAC derivative or a segment thereof comprising at least an origin of replication (ori). The bacteriophage is preferably a λ bacteriophage. The ori can preferably be an ori capable of maintaining the plasmid at single copy.

The pBAC or segment thereof, comprised into the bacteriophage, may further comprise:
a site into which an DNA fragment can be cloned;
at least one pair of inducible excision-mediating sites flanking the site into which the DNA fragment can be cloned, the excision-mediating sites being provided in parallel orientation relative to one another and defining an excisable fragment that comprises the site into which the DNA fragment can be cloned. The pair of inducible excision-mediating sites can be, for example, sites provided in parallel orientation relative to one another (see U.S. Pat No. 5,874,259). The pair of excision-mediating sites are preferably FRT sites. The bacteriophage may further comprises into pair of excision-mediating sites a sequence as shown in SEQ ID NO:45 (according to U.S. Pat. No. 5,874,259).

The pBAC or segment thereof, comprised into the bacteriophage, may further comprise an inducible origin of replication, preferably oriV. Thus oriV may be induced to produce multiple copies of the BAC plasmid (the pBAC is usually present at single copy).

This bacteriophage can comprise one or more of the recombination sites described in the present application. For example, this bacteriophage may comprise at least two recombination sites selected from the following: (a) two recombination sites, wherein either site does not recombine with the other; (b) two lox recombination sites, wherein either site is capable of recombining with each other; (c) two homing endonuclease asymmetric recognition site sequences; (d) two restriction asymmetric endonuclease cleavage site sequences, wherein either site sequence does ligate with the other, recognizable by class IIS restriction enzymes.

The two recombination sites (a) may be selected from the group consisting of attB, attP, attL, attR and derivatives thereof.

The two recombination sites (a) may also be lox recombination sites derivative, which do not recombine with each other.

The two recombination sites (b) are preferably loxP sites.

The two homing endonuclease site sequences (c) are preferably selected from the group consisting of: I-CeuI, PI-SceI, PI-PspI, and I-SceI.

The excision used can be any excision system, included those described in FIG. 3.

The bacteriophage may further comprise at least a background-reducing sequence, for example: a) the ccdB gene; b) the lacZ gene; c) a lox sequence.

It is also provided a method for cloning a nucleic acid of interest or for preparing a bulk nucleic acid library of interest comprising the steps of:
(a) preparing a bacteriophage cloning vector comprising a pBAC (or a pBAC derivative) or a fragment thereof:
(b) inserting a nucleic acid of interest into the bacteriophage cloning vector;
(c) allowing the in vivo or in vitro excision of the plasmid (pBAC or derivative thereof) comprising the nucleic acid insert of interest; and
(d) recovering the BAC plasmid carrying the nucleic acid insert of interest or a library of these BAC plasmids.

The present invention also relates to a kit comprising at least a cloning vector or at least a library of vectors according to the invention.

The present invention will be further explained more in detail with reference to the following examples.

### EXAMPLES

Bacterial Strains

The following not limitative list of bacterial strains were used in the following examples: C600, F⁻ thi-1 thr-1 leuB6 lacYl tonA21 supE44-λ⁻; XL1-Blue-MRA(P2), Δ(mcrA)183 Δ(mcrCB-hsdSMR-mrr)173 endA1 supE44 thi-1 gyrA96 relA1 Jac (P2 lysogen); DB3.1, F⁻ gyrA462 endA Δ(srl-recA) mcrb mrr hdsS20(r_{B}⁻, m_{B}⁻) supE44 ara-14 gaIK2 lacYl proA2 rpsL20 xyl-5 λ leu mt/1; BNN132, e14(McrA) Δ (lac-proAB) thi-1 gyrA96 endal hsdR17 rela1 supE44 [F traD36 proAB lacZ Δ M15] constitutively expressing Cre-recombinase (Elledge et al., 1991, Proc. Natl. Sci. USA, 88:1731-1735); and DH10B, F⁻ mcrA A(mrr-hsdRMS-mcrBC) Φ80 lacZΔM15 ΔlacX74 deoR recA1 endA1 araDl39 (ara-leu)7697 galU galKλ⁻ rpsL nupG (these bacterial strains are all commercially available).

Structure and Nomenclature of λ-FLC Vectors

The basic name of the constructed vectors used in the present description derives from full-length cDNA; the roman numerals indicate: I, general use; II, presence of Gateway sequence (Life Technology); and III, presence of homing endonuclease sites. L and S indicate whether the cloning capacity of the vector better accommodates long (size-selected) or short cDNAs. B, C, D, E, and F indicate the type of stuffer I, as described in FIGS. 1b-f.

Basic Components of λ-FLC Vectors

We constructed a series of λ-based cloning vectors for broad-size directional cloning of full-length cDNAs. These λ-FLC vectors can nominally package inserts of approximately 0.2 to 15.4 kb.

Another benefit of our λ-FLC vectors is that they accommodate cloning and bulk-excision of short and long cDNAs at similar efficiencies within the same library. Then, we adapted these vectors for additional purposes, for example, for selecting very long or full-length cDNAs by using the stuffer II of 5.5 kb (that is a complete size of the construction segment CS of 37.5 kb).

The components used to construct the vectors were assembled to produce several constructs shown in FIGS. 1 and 2.

FIG. 1a illustrates the general scheme for the assembly of the λ-FLC vectors and excision into a plasmid library by using Cre-recombinase or Gateway recombination system.

The basic structure of the λ-based vectors according to the present invention, consists of the left and right λ-arms, which are functionally the same as those of λ-2001 (Karn et al., 1984, Gene, 32:217-224). Between the left and right arms, we inserted a stuffer (stuffer I) and a modified pBluescript or pBAC, flanked on both sides, by two lox P sites for the bulk excision of the plasmid cDNA library, analogous to the structure of λ-PS (Nehls et al., 1994a, as above).

An example of pBluescript construct is shown in FIG. 13 and SEQ ID NO:51.

The calculated size of the λ arms plus the plasmid, but excluding stuffer I (which is substituted with the cDNA in a library) and stuffer II, is about 32 kb. Stuffer II is the “cloning size regulator” and determines the size of the insert, given that the nominal lambda packaging capacity (Zabarovsky et-al., 1993, Gene, 127:1-14). When stuffer II is 5.5 kb long, as in several constructs presented here, the size of the vector, excluding stuffer I, (that is the size of the construction segment CS) is calculated to be 37.5 kb. As reported in Table1, the vector having a stuffer II of 5.5 kb (CS size of 37.5 kb) is particularly useful in selecting long and full-length cDNAs compared to the use of the same vector having a stuffer II of 6 kb (CS size of 38 kb).

Alternative stuffer II elements of 0 and 6.3 kb or even more, were also used to shift the cloning size and collect wide range size of cDNAs.

Type I stuffers (FIGS. 1d-f) can contain the background indicator LacZ and a background-reducing element, such as the ccdB-toxic element or an additional lox P site, which separates the antibiotic resistance gene and the origin of replication during excision (FIG. 1i).

All of the excised plasmids contain conventional forward (Fwd) and reverse (Rev) primer sequences and T7/T3 RNA polymerase promoters, to allow transcriptional sequencing (Sasaki et al., 1998, Proc. Natl. Acad. Sci. USA, 95:3455-3460) and transcription (FIGS. 2g-j, underlined sequences).

In addition, all plasmids can be used to produce single-stranded DNA (ssDNA), and all of them carry the f1(+) origin (Short et al., 1988, as above). When using the f1(+) origin of replication with helper phages such as R408 (Short et al., 1988, as above) to rescue ssDNA, the strand that is rescued is the opposite of the strand represented in FIGS. 2g-j.

In some constructs, we have also introduced cloning or recombination sites such as Gateway sequences flanking RS or the cDNA of interest or placing site sequences for homing endonucleases (New England Biolabs, Inc. also indicated as NEB) for bulk or individual excision of the cloned insert.

### Example 1

### Construction of Vectors

Any vector according to the invention was generated by following standard molecular biology techniques (Sambrook et al., 1989) and using the components shown in Figures. The λ arms (that is the portions at left and right side of Stuffer I) in vectors according to the invention were derived from λ-PS (Nehls et al., 1994a, as above) and were originally described for λ-2001 (Karn et al., 1984, Gene, 32:217-224). Into the XbaI site in the left arm of λ-PS, we inserted a 5.5-kb genomic fragment obtained by PCR amplification of mouse genomic DNA that was cleaved with XbaI and to which was ligated a linker/primer adapter containing an AscI restriction site for later removal or modification of the insert: the linker/primer upper oligonucleotide is: 5″-CTAGGCGCGCCGAGAGATCTAGAGAGAGAG (SEQ ID NO:9); the lower oligonucleotide is: 5′-CTCTCTCTCTAGATCTCTCGGCGC-3′ (SEQ ID NO:10). The upper is also used for PCR amplification.

Before PCR amplification, the genomic DNA also was cleaved with XhoI, SalI, and SfiI to eliminate these sites from the amplified fragment. The amplification and agarose gel-purification steps (Boom et al., 1990, J. Clin. Microbiol., 28:495-503) were repeated 3 times. The 5.5-kb fragment size was chosen as the size regulator (stuffer II) for the λ-FLC-I-B vector, and its derivatives were created by cloning similarly obtained fragments of approximately 4.5 to 5.5 kb and we verified that inserts as short as 0.5 kb were clonable. In addition, the sequences of the polylinkers (sequences as appears in the excised plasmids of FIG. 2) and stuffer I (FIG. 1) were changed to accommodate directional cloning (according to Standard molecular biology techniques, for example Sambrook et al.), basically, restriction digestion, followed by re-ligation (T4 DNA ligase) with linker having the desired sequences which are inserted between the previous fragments of the phage. The 10-kb stuffer I (FIG. 1b) was obtained from λ-PS (Nehls et al., 1994a, as above). The 3-kb shorter fragment of the stuffer (FIG. 1c) was obtained by digesting the 10-kb stuffer I with XhoI and SalI. Subsequently, we amplified this 3-kb with the primers 5′-GAGAGACTCGAGGTCGACGAGAGAGGCCCGGGCGGCCGCGATCGCGGCCGGCCAGTCTTTAATTAACT-3′ (SEQ ID NO:11) and 5′-GAGAGAGGATCCGAGAGAGGCCAGAGAGGCCATTTAAATGCCCGGGCTGCAGGAATTCGATAT-3′ (SEQ ID NO:12) to add several restriction sites to the 3-kb stuffer (FIG. 1c). To this modified stuffer (FIG. 1c), we inserted the blunt-ended LacZ cassette into the SwaI site. Then, we restricted the modified stuffer with SfiI and inserted the ccdB gene as a triple ligation to obtain the stuffer I in FIG. 1e. The ccdB gene was obtained by PCR amplification of the template pDEST-C, which can be propagated in E. coli DB3.1 (Life Technologies); the primer pairs were 5′-GAGAGAGCGGCCGCCCGGGCCATTTAAATCCGGCTTACTAAAAGCCAGA-3′ (SEQ ID NO:13) and the reverse primer 5′-AGCGGATAACAATTTCACACAGGA-3′ (SEQ ID NO:14)(as in pBluescript, Stratagene), and 5′-GAGAGAGGCCTCTCTGGCCACTAGTCTGCAGACTGGCTGTGTATA-3′ (SEQ ID NO:15) and the forward primer 5′-TGTAAAACGACGGCCAGT-3′ (SEQ ID NO:16). The LacZ cassette was obtained by digesting a pUC18 with NaeI and AflII and then blunting the appropriate fragment by using the Klenow fragment of DNA polymerase before cloning.

Lox P, attB, and the modified polylinker sequences were prepared by annealing complementary oligonucleotides.

The stuffer I of FIG. 1e, after blunting the SalI and BamHI restriction sites, was dimerized by ligation with DNA ligase (New England Biolabs) to obtain the stuffer in FIG. 1d. The stuffer in FIG. 1f was obtained by PCR amplifying the stuffer in FIG. 1c with a primer containing the Lox P site, 5′-GAGAGAGGATCCAGAGAGATAACTTCGTATAATGTATGCTATACGAAGTTATGAGAGAGGCCAGAGAGGCCATTTAA-3′ (SEQ ID NO: 17)(on the BamHI side), and the primer 5′-GAGAGACTCGAGGTCGACGAGAGAGGCCCGGGCGGCCGCGATCGCGGCCGGCCAGTCTTTAATTAACT-3′ (SEQ ID NO: 18)(on the SalI side). After purification (according to Boom et al., 1990, as above) and restriction digestion, this fragment was ligated with DNA ligase (according to Sambrook et al., 1989) to the ccdB fragment to yield the stuffer in FIG. 1f.

The plasmids obtained after excision (described later) are derivatives of pBluescript+ (Stratagene) or pBAC. The pDEST-C vector (Life Technologies) is the acceptor plasmid of the LxR reaction (Gateway System, Life Technologies) and, after excision, produces pFLC-DEST (FIG. 2.j). pDEST is prepared from pBluescript II SK+ (Stratagene) by removal of the polylinker by digesting the pBluescript II SK+ with the restriction enzymes SacI and KpnI. Then, blunting the cleaved extremities with T4 DNA polymerase (according to Sambrook et al., 1989). The rfB II cassette (purchased by Life Technologies) comprising the ccdB gene was then inserted and ligated into the cleaved plasmid following the instruction of Gateway Cloning System Manual, Version 18.4, Life Technologies. The ligated plasmid vector was then cleaved with BssHI restriction enzyme and the cleaved fragment inverted (that is rotated of 180 degrees) and re-entered into the vector (according to known methodologies, Sambrook et al, 1989).

The pDEST-C vector was used in the same way as is pDEST12.2 (Catalog and Instruction Manual, Gateway™ Cloning Technology, GIBCOBRL®, Life Technologies®).

The λ-FLC-I-B vector was in general used as starting point for the construction of the other vectors according to the invention.

λ-FLC-I-E was obtained by substituting the stuffer in FIG. 1e for that of λ-FLC-I-B. λ-FLC-I-L-B was obtained by removing stuffer II from λ-FLC-I-B, and λ-FLC-I-L-D was created by substituting the stuffer shown in FIG. 1e for that of λ-FLC-I-B. λ-FLC-II-C was obtained by joining a modified pBluescript II KS+ (purchased from Stratagene) with a stuffer like that in FIG. 1c; the rest of the vector was as in λ-FLC-I-B. λ-FLC-III-F was created by inserting a construct containing the plasmid sequence and stuffer I of FIG. 1f (the construct is shown FIG. 2d) into λ-FLC-I-B-derived phage arms (including the 5.5-kb stuffer II) in the same way as described in the example “preparation of λ-FLC-III-C (but introducing the stuffer 1f instead of the stuffer 1c). The vector λ-FLC-III-F was also prepared as shown in FIG. 7. λ-FLC-III-L-D was obtained from λ-FLC-III-F by first substituting the stuffer I of FIG. 1f with the one of FIG. 1d, followed by deletion of stuffer II. λ-FLC-III-S-F was obtained by ligating (using DNA ligase, as described in Sambrook et al., 1989) the concatenated arms from λ-FLC-I-B (devoid of stuffer II) with a 6.3 Kb long stuffer II and the “plasmid+stuffer I” derived from λ-FLC-III-F. Vector λ-FLC-III-E was prepared in the same ways as described for λ-FLC-III-F (and λ-FLC-III-C) introducing the stuffer 1e instead of the stuffer 1c or 1f; with “stuffer 1e” it is intended the stuffer I of FIG. 1e, and the like for the other stuffers). Vectors comprising a pBAC or pBAC derivative can be prepared as shown in Example 20 and according to FIGS. 9-12.

### Example 2

### Preparation of λ-Arms for Cloning

The final λ-DNA constructs were prepared by using standard methods (Sambrook et al., 1989) or the Lambda Maxi Prep Kit (#12562, Qiagen). The cohesive termini (cos ends) of 10 μg of λ-DNA were annealed by incubating for 2 h at 42° C. in 180 μl 10 mM Tris.Cl (pH 7.5)/10 mM MgCl₂. We then added 20 μL 10× ligation buffer and 400 U T4 ligase (New England Biolabs) and incubated the mixture for 5 h at room temperature. The ligase was inactivated by incubating for 15 min at 65° C.

At this point, the λ-DNA was digested with the required restriction enzymes (as described below; all purchased from New England Biolabs) in 3 steps because of the different concentrations of NaCl needed. For the first step, restriction was done in 50 mM NaCl by the addition of 2 μL 5 M NaCl, 6 U FseI, and 8 U PacI for each vector. The sample (the vector) was incubated for 4 h or overnight at 37° C. The second step was done in 100 mM NaCl by adding 2 μL 5 M NaCl, 30 μL 10×NEB 3 buffer, 270 μL H₂O, and 20 U SwaI to the previous reaction and incubating for 2 h at room temperature. After this step, the reaction tube was heated for 15 min at 65° C. Finally, the third step was done in 150 mM NaCl by adding 5 μL 5 M NaCl, 40 U XhoI (in the cases of the μ-FLC-I and -III vectors, to reduce the background by reducing the size of the E. coli genomic DNA fragments; and for the μ-FLC-II vectors, to create the cloning site), 40 U SalI, and 40 U BamHI to the heat-inactivated reaction and incubating for 4 h at 37° C. For λ-FLC-II vectors, the SalI may be omitted or may be used to generate an alternative to the XhoI cloning site. The FseI, PacI and SwaI step are omitted for the λ-FLC-I-B, which does not carry these sequences.

After restriction, the DNA was purified by proteinase λ treatment in the presence of 0.1% SDS and 20 mM EDTA, extracted with 1:1 phenol/chloroform and chloroform, and precipitated with ethanol (Sambrook et al., 1989). To avoid problems during resuspension, the DNA concentration did not exceed 20 μg/mL.

After careful resuspension for at least 30 min, the digested DNA was separated in a 0.66% low-melting point agarose gel (Seaplaque®, FMC) according to the followings steps. The wells were in the middle of the gel. After electrophoresis for 1.5 h at 8 V/cm, the DNA fragments of the StyI-digested λ-DNA that were shorter than 19 kb were cut from the gel and discarded (step 1). Then, the electrophoresis buffer 1×TBE (electrophoresis buffer Tris-Borate-EDTA; see Sambrook et al., 1989) was replaced with fresh buffer, and the DNA remaining in the gel was electrophoresed in the opposite direction at 8 V/cm for 2.5 h. Then the DNA shorter than 19 kb again was discarded (step 2). The buffer was changed again. To condense the region containing the λ-arm DNA to decrease reaction volumes, the DNA remaining in the gel was electrophoresed at 8 V/cm for 30 min in the same direction as for step 1. Finally, the portion of the gel containing the λ-arm DNA was removed (step 3), the gel was equilibrated with TE buffer (Sambrook et al., 1989), and the λ-arms were purified and checked as described (Carninci and Hayashizaki, 1999, Methods Enzymology, 303:19-44) by using β-agarase (New England Biolabs). We typically recovered 30% to 50% of the starting λ-DNA. The purified λ-arms were stored indefinitely in single-use aliquots at −80° C. or at +4° C. for up to 1 week. A typical cloning efficiency was 1-2×10⁷ pfu/μg λ-FLC-I-B vector with a test insert of 6 kb and less than 1% background of non-recombinant clones.

### Example 3

### Preparation of λ-FLC-I-B

λ-PS vector has been cleaved using BamHI restriction enzymes and stuffer I inserted using a left linker adapter comprising two complementary oligonucleotides: upper oligonucleotide 5′-GATCAGGCCAAATCGGCCGAGCTCGAATTCG-3′ (SEQ ID NO: 19) and lower oligonucleotide 5′-TCGAGAATTCGAGCTCGGCCATTTGGCCT-3′ (SEQ ID NO:20), and a right linker adapter comprising two complementary oligonucleotides:

(SEQ ID NO:21)

upper oligonucleotide
5′-GATCAGGCCCTTATGGCCGGATCCACTAGTGCGGCCGCA-3′
and

(SEQ ID NO:22)

lower oligonucleotide
5′-TCGATGCGGCCGCCTAGTGGATCCGGCCATAAGGGCCT-3′.

Each one of two oligonucleotides of the left adapter, that is SEQ ID NO:19 and SEQ ID NO:20 was treated with Kinase with cold ATP for 20 min at 37° C. as follows: 1 μg of each oligonucleotide, 1 μl of ATP 5mM, 2 μl of PNK buffer (New England Biolabs), 0.5 μl of PNK (Polynucleotide Kinase; New England Biolabs), and water up to 20 μl. The obtained products were the two complementary oligonucleotides 5′-phosphorilated. The two oligo (SEQ ID NOS:19 and 20) solutions were mixed together and NaCl added to a final concentration of 100 mM. The mixer was incubated 15 min at 65° C. and then for 10 min at 45° C. to carry out the annealing. The annealed oligos were diluted at the concentration 0.5 ng/μl suitable for cloning. The same procedure was carried out for the oligo pair (SEQ ID NOS: 21 and 22) which were also annealed forming the right adapter.

200 ng of λ-PS vector above cleaved with BamHI (that is the left and the right arms) were mixed with 0.4 ng of the left adapter and 0.4 ng of the right adapter, and 60 ng of the stuffer I, in a final volume of 5 μl. The ligation was carried out overnight (alternatively the ligation can also be carried out for 2 hours and 16° C.). The ligated vector/adapters/stuffer I was packaged according to the methodologies known in the art Sambrook et al., 1989).

A stuffer II of 5.5-kb genomic fragment obtained by PCR amplification of mouse genomic DNA that was cleaved with XbaI was ligated at both extremities with a linker/primer adapter containing an AscI restriction site for later removal or modification of the insert. The linker/primer upper oligonucleotide is: 5″-CTAGGCGCGCCGAGAGATCTAGAGAGAGAG (SEQ ID NO:9); the lower oligonucleotide is:

| | |
|---|---|
| 5′-CTCTCTCTCTAGATCTCTCGGCGC-3′. | (SEQ ID NO:10) |

The stuffer II with the adapter was introduced into the XbaI site in the left arm of λ vector above prepared, obtaining the vector λ-FCL-I-B.

From this vector after the excision with in vitro Cre-lox recombinase (as described later), the plasmid pFLC-I-b (the plasmid of FIG. 2g comprising the stuffer I of FIG. 1b) was obtained.

### Example 4

### Preparation of λFLC-III-C

Plasmid pFLC-I-b, obtained from excision of λ-FLC-I-B as described above, was used as template and amplified by PCR. The primers used were: T7 Rev (56 mer) 5′-GTGTGATATCGCCCTATAGTGAGTCGTATTACATAGCTGTTTCCTGTGT GAAATTG-3′ (SEQ ID NO:23) and T3 Fwd (70 mer) 5′-GAGAGATATCTTTGTTCCCTTTAGTGAGGGTTAATTGCGCGCAATTCA CTGGCCGTCGTTTTACAACGTC-3′ (SEQ ID NO:24) obtaining the linear “product 1”.

Plasmid pFLC-IIc was used as a template and amplified by PCR. The primers used were: FLCIIX2 (68 mer) 5′-GAGAGACTCGAGGTCGACGAGAGAGGCCCGGGCGGCCGCGATCGCG GCCGGCCAGTCTTTAATTAACT-3′ (SEQ ID NO:25) and primer FLCIIB2 (63 mer) 5′-GAGAGAGGATCCGAGAGAGGCCAGAGAGGCCATTTAAATGCCCGGGC TGCAGGAATTCGATAT-3′ (SEQ ID NO:26). The product of this PCR was cleaved with XhoI and BamHI restriction enzyme obtaining a linear fragment of 3 bk. This fragment was used as template for PCR amplification with the primers: 5′ I-CeuI-SalI (59 mer) 5′-GTGTAACTATAACGGTCCTAAGGTAGCGAGTCGACGAGAGAGGCCCG GGCGGCCGCGAT-3′ (SEQ ID NO:27) and 3′PI-SceI-BamHI (67 mer) 5′-GCATCTATGTCGGGTGCGGAGAAAGAGGTAATGAAATGGCAGGATCCGA GAGAGGCCAGAGAGGCCA-3′ (SEQ ID NO:28), obtaining the linear “product 2”.

The “product 2” was then phosphorilated with PNK-polynucleotide kinase and gamma-ATP according to Sambrook et al., 1989.

Then, the “product 1” was cleaved with the EcoRV restriction enzyme and the fragment obtained was ligated (according to the standard methodology, Sambrook e al., 1989) with the “product 2” prepared as above. A (circular) plasmid indicated as “product 3” was obtained.

The plasmid “product 3” was used as template and amplified by PCR using the primers: XbaI-LoxP Tag primer 3F (69 mer) 5′-GAGAGTCTAGATAACTTCGTATAGCATACATTATACGAAGTTATAAATC AATCTAAAGTATATATGAGT-3′ (SEQ ID NO:29) and XbaI-LoxP Tag primer 3R (69 mer) 5′-GAGAGTCTAGATAACTTCGTATAATGTATGCTATACGAAGTTATAAAAC TTCATTTTTAATTTAAAAGG -3′ (SEQ ID NO:30) obtaining a linear product, which was then cleaved with XbaI restriction enzyme, obtaining the linear “product 4”.

A λ-FLC-I-B was cleaved with XbaI restriction enzyme, then purified with electrophoresis according to the standard methodology (Sambrook, et al., 1989) and the resulting λ left arm, λ right arm, and stuffer II were recovered from the purification by electrophoresis. 200 ng of λ left arm, 90 ng of λ right arm, 55 ng of Stuffer II, and 60 ng of the “product 4” were ligated overnight according to the standard methodology (Sambrook et al., 1989). The obtained vector λ-FLC-III-C was packaged according to the methodologies known in the art (Sambrook et al., 1989).

By treatment with Cre-recombinase, the in vitro cre-lox recombinase excision was carried out and the plasmid pFLC-III-c (plasmid of FIG. 2i comprising the stuffer I of FIG. 1c)) obtained.

Other λ-FLC vectors can be prepared starting from λ-FLC-III-C vector. For example, vector λ-FLC-III-F or λ-FLC-III-E can be prepared by substituting the stuffer Ic of λ-FLC-III-C with the stuffer If or Ie, respectively.

### Example 5

### Preparation of λ-FLC-II-C

pBluescript II SK+ (purchased from Stratagene) was digested with Kpn I and Not I. The large fragment was separated by agarose gel electrophoresis and purified.

λ-FLC-I-B was digested with XhoI and SalI and blunted by T4 DNA polymerase, according to standard methodology (Sambrook et al., 1989). A 3 kb fragment was separated by agarose gel and purified.

Then three double stranded linkers (AttB1, AttB2 and LoxP) were synthesized as follows.

AttB1 linker: upper oligonucleotide is 5′-CGGGCCACAAGTTTGTACAAAAAAGCAGGCTCTCGAGGTCGACGAGA GGCCAGAGAGGCCGGCCGAGATTAATTAA-3′ (SEQ ID NO:31), lower oligonucleotide is 5′-TTAATTAATCTCGGCCGGCCTCTCTGGCCTCTCGTCGACCTCGAGAGC CTGCTTTTTTGTACAAACTTGTGGCCCGGTAC-3′ (SEQ ID NO:32).

AttB2 linker: upper oligonucleotide is 5′-GGCCATGACGGCCGAGAGATTTAAATGAGAGAGGATCCACCCAGCTT TCTTGTACAAAGTGGTCTAGACCTCTCTTGG-3′ (SEQ ID NO:33), lower oligonucleotide is 5′-GAGGTCTAGACCACTTTGTACAAGAAAGCTGGGTGGATCCTCTCTCAT TTAAATCTCTCGGCCGTCATGGCC-3′ (SEQ ID NO:34).

LoxP linker: upper oligonucleotide is 5′-CCGCATAACTTCGTATAGCATACATTATACGAAGTTATGC-3′ (SEQ ID NO:35), lower oligonucleotide is 5′-GGCCGCATAACTTCGTATAATGTATGCTATACGAAGTTATGCGGCCAA GA-3′ (SEQ ID NO:36).

The lower strand of attB2 linker and the upper strand of LoxP linker were phospohorylated by using polynucleotide kinase PNK; New England Biolabs) according to how described above in the preparation of λ-FLC-I-B.

The two oligos (SEQ ID NO:31 and 32) solutions were mixed together and NaCl added to a final concentration of 100 mM. The mixer was incubated 15 min at 65° C. and then for 10 min at 45° C. to carry out the annealing. The annealed oligos were diluted at the concentration 0.5 ng/μl suitable for cloning. The same procedure was carried out for the oligo pairs (SEQ ID NO: 33 and 34; and for SEQ ID NO:35 and 36) which were annealed respectively. AttB2 linker (0.5 ng) and LoxP linker (0.5 ng) were mixed and ligated in the volume of 5 μl. The tube was incubated at 16° C. After 20 min, attB1 linker (0.5 ng), pBluescript cleaved with KpnI and NotI (25 ng) and the 3 kb fragment from λ-FLC-I-B (25 ng) were added in the tube in the volume of 10 μl. Then, it was incubated overnight at 16° C. obtaining a ligation solution comprising a plasmid comprising the ligated fragment. The ligation solution comprising a plasmid was then introduced by electrophoresis into DH10B cells and plated on a medium. Plasmids was prepared from the recombinant cells. The cells were lysed and the plasmids cleaved with XbaI and a plasmid fragment was obtained “fragment 1”.

A junction linker was prepared, having an upper oligonucleotide: 5′-GGCCATGAGAT-3′ (SEQ ID NO:37), and a lower oligonucleotide is: 5′-CTAGATCTCAT-3′ (SEQ ID NO:38). These two oligonucleotide were annealed and the “fragment 2” obtained.

λ-FLC-I-B was cut with NotI and a 26 kb fragment was separated with agarose gel and purified “fragment 3”.

A 9 kb fragment was also prepared by cleavage with XbaI of λ-FLC-I-B “fragment4”.

These “fragments 1-4” (26 kb left arm, the junction linker, stuffer-plasmid, 9 kb right arm) were ligated in the volume of 5 μA. The ligation solution was packaged and amplified obtaining the vector λ-FLC-II-C. These steps were carried out according to standard procedures (Sambrook et al., 1989).

From the vector λ-FLC-II-C after in vitro excision with Cre-recombinase (see later), the plasmid pFLC-II-c (the plasmid of FIG. 2j comprising the stuffer I of FIG. 1c) was obtained.

### Example 6

### Preparation of λ-FLC-III-F

A λ-FLC-III-F vector can be prepared as described at the end of Example 4, however, other methods of preparation are also possible. One alternative way of preparation of λFLC-III-F, which will be described in the present example is represented in FIG. 7.

To obtain lambda arms and stuffer II (5.5 kb), the cohesive termini of 10 μg of λ-FLC-I-B were annealed by incubating for 2 h at 42° C. in 180 μl 10 mM Tris.Cl (pH 7.5)/10 mM MgCl₂. We then added 20 μL 10× ligation buffer and 400 U T4 DNA ligase (New England Biolabs) and incubated the mixture for 5 h at room temperature. The ligase was inactivated by incubating for 15 min at 65° C. The concatemerized λ-FLC-I-B was digested with 30 units of Xba I (NEB) in 1× manufactures recommendation buffer. The tube was incubated for 2 h at 37° C.

After restriction, λ-FLC-I-B/XbaI DNA was purified by proteinase K (Qiagen) treatment in the presence of 0.1% SDS and 20 mM EDTA, extracted with 1:1 phenol/chloroform and chloroform, and precipitated with ethanol (Sambrook et al., 1989). To avoid problems during resuspension, the DNA concentration did not exceed 20 μg/mL.

After careful resuspension for at least 30 min, the digested DNA was separated in a 0.6% low-melting point agarose gel (Seaplaque®, FMC) for 1.5 h at 8 V/cm. The portion of the gel containing the 29 kb λ DNA (ligation product between L-arm and R-arm) and 5.5 kb stuffer II were cut out and equilibrated with TE buffer (Sambrook et al., 1989). The DNAs were purified and checked as described (Carninci and Hayashizaki, 1999, Methods Enzymology, 303:19-44) by using β-agarase (New England Biolabs).

3 μg of pBS II SK+ (Stratagene) was digested with 9 unit of Bss HII (NEB) at 37° C. for 2 h and dephosphorylated by CIP (Takara, Japan) (Sambrook et al., 1989, standard technique).

To introduce homing nuclease sites (I-CeuI and PI-SceI) into pBS II SK+, double strand, an I-CeuI/PI-SceI adaptor oligonucleotide comprising an oligonucleotide up adaptor strand: 5′-pCGCGCTAACTATAACGGTCCTAAGGTAGCGAGTCGACGAGAGAGAG AGGATCCATCTATGTCGGGTGCGGAGAAAGAGGTAATGAAATGGCAG-3′ (SEQ ID NO:39) and an oligonucleotide down adaptor strand: 5′-pCGCGCTGCCATTTCATTACCTCTTTCTCCGCACCCGACATAGATGGATC CGAGAGAGAGAGTCGACTCGCTACCTTAGGACCGTTATAGTTAG-3′) (SEQ ID NO:40) was prepared (according to standard technique), and ligated with pBS II SK+/BssHII (NEB)/CIP (Takara, Japan).

pBS II SK+/BssHII/CIP and I-CeuI/PI-SceI adaptor were ligated, by mixing 100 ng of pBS II SK+/BssHII/CIP, 2 ng of I-CeuI/PI-SceI adaptor, 400 unit T4 DNA ligase, 1× ligation buffer in a total volume of 5 μl. The tube was incubated overnight at 16° C.

The ligation products were introduced into DH10B and cultured. The clones containing the proper plasmid were selected by preparing plasmid and restriction using I-CeuI (Sambrook et al., 1989, standard technique). Then the I-CeuI/PI-SceI adaptor was substituted with Stuffer If (the stuffer I of FIG. 10 described as following.

3 μg of plasmids comprising I-CeuI/PI-SceI adaptor were digested with 9 units of Sal I and 9 units of Bam HI in 30 μl. To remove the SalI-BamHI short fragment, the plasmid/SalI and BamHI were separated in a 0.6% low-melting point agarose gel (Seaplaque®, FMC) for 1.5 h at 8 V/cm. The 3 kb DNA was cut out and equilibrated with TE buffer (Sambrook et al., 1989). The 3 kb DNA were purified and checked as described (Carninci and Hayashizaki, 1999, Methods Enzymology, 303:19-44) by using β-agarase (New England Biolabs). We typically recovered 30% to 50% of the starting DNA.

100 ng of the plasmid DNA and 140 ng of stuffer If were ligated with 400 unit T4 DNA ligase, 0.5 μl of 10× ligation buffer in a total volume of 5 μl. The tube was incubated overnight at 16° C.

The ligation products were introduced into DH10B and cultured. The clones containing the proper plasmid were selected by preparing plasmid and restriction using BamHI and SalI (Sambrook et al., 1989, standard technique).

In the next step loxP sites were introduced into the vector between amp^{r} gene and ori. LoxP was introduced by PCR using XbaI-LoxP Tag primer 3F (69 mer) having the sequence: 5′-GAG-AGT-CTA-GAT-AAC-TTC-GTA-TAG-CAT-ACA-TTA-TAC-GAA-GTT-ATA-AAT-CAA-TCT-AAA-GTA-TAT-ATG-AGT-3′ (SEQ ID NO:41) and XbaI-LoxP Tag primer 3R (69 mer) having the sequence: 5′-GAG-AGT-CTA-GAT-AAC-TTC-GTA-TAA-TGT-ATG-CTA-TAC-GAA-GTT-ATA-AAA-CTT-CAT-TTT-TAA-TTT-AAA-AGG-3′ (SEQ ID NO:42) (according to standard technique).

Using 3 μg of the resulting PCR product (7.2 kb), the PCR product was digested with 9 units of XbaI at 37° C. for 1 h (Sambrook et al.,). To remove short DNA fragment resulting from PCR product/XbaI, the digested product was separated in a 0.6% low-melting point agarose gel (Seaplaque®, FMC) for 1.5 h at 8 V/cm. The 7.2 kb DNA was cut out and equilibrated with TE buffer (Sambrook et al., 1989). The 7.2 kb DNA were purified and checked as described (Carninci and Hayashizaki, 1999, Methods Enzymology, 303:19-44) by using β-agarase (New England Biolabs).

The 7.2 kb PCR product, the purified arms and stuffer II (5.5 k) were ligated in the ratio of 25 ng: 100 ng: 19 ng with 400 units of T4 DNA ligase (Sambrook et al., 1989).

The ligation solution was packaged and amplified obtaining the vector λ-FLC-III-F. These steps were carried out according to standard procedures (Sambrook et al., 1989).

### Example 7

### Preparation of λ-FLC-III-E

The λ-FLC-III-E vector can be prepared by substituting the stuffer I of other FLC-III vectors with the stuffer Ie.

In the present example, λ-FLC-III-E was obtained by substituting the stuffer If of the λ-FLC-III-F vector prepared in Example 6 with the stuffer Ie (i.e. the stuffer I of FIG. 1e) according to the following steps.

The cohesive termini of 10 μg of λ-FLC-III-F were annealed by incubating for 2 h at 42° C. in 180 μl 10 mM Tris.Cl (pH 7.5)/10 mM MgCl₂. We then added 20 μL 10× ligation buffer and 400 U T4 DNA ligase (New England Biolabs) and incubated the mixture for 5 h at room temperature. The ligase was inactivated by incubating for 15 min at 65° C.

At this point, the concatemerized λ-FLC-III-F was digested with the required restriction enzymes, by adding 30 units of BamHI, 30 units of SalI and 40 μl 10× BamHI buffer (all purchased from New England Biolabs) in a total volume of 400 μl. The tube was incubated for 2 h at 37° C.

After restriction, the DNA was purified by proteinase K (Qiagen) treatment in the presence of 0.1% SDS and 20 mM EDTA, extracted with 1:1 phenol/chloroform and chloroform, and precipitated with ethanol (Sambrook et al., 1989). To avoid problems during resuspension, the DNA concentration did not exceed 20 μg/mL.

After careful resuspension for at least 30 min, the digested DNA was separated in a 0.6% low-melting point agarose gel (Seaplaque®, FMC) for 1.5 h at 8 V/cm. The portion of the gel containing the λ DNA was cut out and equilibrated with TE buffer (Sambrook et al., 1989). The λDNA were purified and checked as described (Carninci and Hayashizaki, 1999, Methods Enzymology, 303:19-44) by using β-agarase (New England Biolabs). We typically recovered 30% to 50% of the starting λ-DNA.

To obtain stuffer Ie (FIG. 1e), 10 μg of λ-FLC-I-E were digested with 30 units of BamHI, 30 units of SalI in 200 μl 1×BamHI buffer. The tube was incubated for 2 h at 37° C.

After restriction, the 5 kb DNA fragment was separated in a 0.6% low-melting point agarose gel (Seaplaque®, FMC) for 1.5 h at 8 V/cm. The 5 kb DNA (stuffer Ie) was cut out and equilibrated with TE buffer (Sambrook et al., 1989). The 5 kb DNA were purified and checked as described (Carninci and Hayashizaki, 1999, Methods Enzymology, 303:19-44) by using β-agarase (New England Biolabs). We typically recovered 30% to 50% of the starting DNA.

The λ-FLC-III-F having the stuffer If removed, and stuffer Ie (prepared as above) were ligated (the ratio was 210 ng to 30 ng) by mixing with 400 units T4 DNA ligase in 10 ul of 1× ligation buffer (NEB). The tube was incubated overnight at 16° C.

The ligation solution was packaged and amplified obtaining the vector λ-FLC-III-E. These steps were carried out according to standard procedures (Sambrook et al., 1989).

### Example 8

### Preparation of pDEST-C

pBluescript II SK+ (purchased from Stratagene) was cleaved with SacI and KpnI restriction enzymes followed by blunting with T4 DNA polymerase (Sambrook et al., 1989) and two fragments were obtained. The short fragment was removed by agarose gel electrophoresis and the long fragment purified and recovered. The purified long fragment was ligated with RfB cassette overnight at 16° C. according to standard methodology (Sambrook et al. 1989) and introduced into DH10B cells by electroporation (Sambrook et al. 1989). Recombinant clone was amplified and plasmid extracted (pDEST-A) In order to invert the BssHII fragment in pDEST-A, pDEST-A was cut with BssHII restriction enzyme and then extracted by using phenol/chloroform and precipitated by ethanol (Sambrook et al., 1989) and two fragments were obtained. These two fragments, digestion products of pDEST-A, were ligated overnight at 16° C. by inverting the RfB cassette of 180 degrees (Sambrook et al., 1989) and the obtained plasmid introduced into DH10B cells by electroporation. The clone having the fragment inverted was selected (pDEST-C) by restriction mapping (Sambrook et al. 1989).

### Example 9

### Preparation of pFLC-DEST

λ-FLC-II-C and pDONR201 (Life Technologies) were recombined by BP clonase (Life Technologies). Then the recombination vector was mixed with pDEST-C and recombined by LR clonase. The reaction solution was introduced into DH10B cells by electroporation and the recombinant clone selected on LB plate containing ampicillin. Recombinant cells were amplified and the plasmid (pFLC-DEST) was prepared.

### Example 10

### Preparation of Purified pFLC-III-f

100 ng of λ-FLC-III-F were treated with 1 U Cre-recombinase (in vitro cre-lox mediated recombinase) at 37° C. for 1 hour in 300 μl, and the FLC-III-f plasmid was excised. The plasmid was then extracted with phenol/chloroform, and chloroform, and precipitated with ethanol (according to Sambrook et al., 1989). The recovered plasmids were electroporated into DH10B (Life Technologies) at 2.5 kb/cm. The cells were spread on LB agar containing ampicillin, X-gal (Sambrook et al., 1989) and cultured overnight at 37° C. Blue colony from LB plate containing ampicillin were picked up and plasmids prepared using QIAGEN kit.

The plasmids were digested-with restriction enzymes (I-CeuI, PI-Sce I) according to the following steps.

First restriction step: a solution of 20 μl of 10×I-Ceu I buffer, 20 μl of 10×BSA and 3U of I-Ceu I (total volume 200 μl) was prepared in a tube and incubated for 5 hour at 37° C.

Second step of restriction: 22.5 μl of 10×PI-Sce I buffer and 3 U PI-Sce I were added and the obtained solution incubated for 5 hour at 37° C. After this step, the tube was heated for 15 min at 65° C. Then, the digested DNA was purified by proteinase K treatment (Sambrook et al., 1989), extracted with phenol/chrolofolm, chroloform,and prepicipated with ethanol (as described in Sambrook et al., 1989). After careful resuspension, the digested DNA was separated in 0.8% low melting agarose gel as follows. After electrophoresis for 1.5 hours at 50V, the DNA fragments (2.9 kb) were cut off from gel and recovered. They were purified with QIAGEN QIAquick Gel Extraction kit and then used for the ligation.

### Example 11

### Preparation of cDNA and Cloning

Full-length cDNAs were prepared as described (Carninci and Hayashizaki, 1999, as above; Carninci et al., 1997, DNA Res., 4:61-66) and normalized and/or subtracted (Carninci et al., 2000, Genome Res., 10:1617-1630) before cloning. After digestion with 25 U BamHI (New England Biolabs)/μg cDNA (to cleave the 3′ end) and 25 U XhoI (Fermentas Vilnius, Lithuania)/μg cDNA (to cleave the 5′ end), the cDNA was treated with 1.3 U thermosensitive shrimp alkaline phosphatase (SAP; Amersham Pharmacia Biotech)/μg cDNA to avoid concatenation and chimerism of cDNAs, which are concerns when working with large-capacity cloning vectors. Then the cDNA was treated with proteinase K, extracted with phenol/chloroform, and applied to a CL-4B spin column (Amersham Pharmacia Biotech). The purified cDNA was ethanol-precipitated (Carninci and Hayashizaki, 1999, as above) or size-fractionated. Normalization/subtraction was not used for cDNA that was size-fractionated by using an agarose gel. This process was similar to that used in the isolation of the λ arms of the vectors: the direction of electrophoresis was inverted after short fragments were run out of the gel (we changed the buffer before resuming the electrophoresis). cDNA was isolated from the gel either by using β-agarase (New England Biolabs) as described or by binding in the presence of 7 M guanidine-Cl to double-acid-washed and size-fractionated diatomaceous earth (Sigma) essentially as described (Boom et al., 1990, J. Clin. Microbiol., 28:495-503).

cDNA and vectors were always ligated(according to Carninci and Hayashizaki, 1999, Methods Enzymology, 303:19-44) at an equimolar ratio in a 5-μL reaction containing T4 DNA ligase (New England Biolabs). The quantity of cDNA was estimated by the radioactivity incorporated during synthesis of the first and second strands (Carninci and Hayashizaki, 1999, as above). The cloning sites on the vectors were the SalI (cohesive ends with XhoI) and BamHI sites, except that XhoI and BamHI sites were used for the λ-FLC-II-C vector.

cDNA sequencing was performed as described (Shibata K., et al., 2000, Genome Res., 10:1757-1771), and sequence analysis and clustering were performed as described (Konno et al., 2001, Genome Res., 11:281-289).

### Example 12

### Bulk Excision of cDNA Libraries

I) In Vivo, Solid-Phase Excision (State of the Art)

cDNA libraries were amplified in E. coli C600 cells. Approximately 1-5×10⁴ pfu were plated on 150-mm dishes of LB-agar, topped with LB-agar containing 10 mM MgSO₄, and grown overnight to confluence (Sambrook et al., 1989, as above). Subsequently, phage particles were eluted with SM-buffer and titered. Then, BNN132 cells were grown overnight in LB-broth plus 10 mM MgSO₄. Cells were pelleted, resuspended in 10 mM MgSO₄, and immediately infected with the phage library, which was converted in vivo to a plasmid DNA library and plated on LB-ampicillin plates.

II) In Vivo, Liquid-Phase Excision

Up to 5×10¹⁰ phage particles prepared as above were used to infect 10 mL of overnight-grown BNN132 cells (OD₆₀₀=˜0.5) after pelleting and resuspending in 10 mM MgSO₄, which were then cultured in 90 LB medium supplemented with 100 μg/ml of ampicillin. After 1, 2 or 3 h at either 30° C. or 37° C., the cultures were stopped, and we extracted the plasmid by using the Wizard Plus Midiprep DNA Purification System (Promega). The plasmid library was electroporated into DH10B cells (Life Technologies) at 2.0 Kv/cm, which are suitable for sequencing operations as described (Shibata K., et al., 2000, as above).

III) In Vitro Cre-Lox-Mediated Excision

Phage cDNA libraries were amplified in C600 cells as described. We isolated the library phage DNA from the amplified phage solution by using the Wizard Lambda Preps DNA Purification System (Promega). We converted one fourth of the obtained phage DNA to plasmid by treating with 1 U Cre-recombinase at 37° C. for 1 h in 300 μL as recommended (Novagen), and then purified (proteinase K treatment, phenol/chloroform extraction and ethanol precipitation, according to Sambrook et al., 1989). The bulk-excised plasmid libraries were electroporated into DH10B cells (Life Technologies) at 2.0 kV/cm.

IV) Gateway-Mediated Bulk-Excision (“Indirect”) Protocol

We mixed 16 ng library phage DNA, 300 ng pDONR201(Instruction Manual, Gateway Cloning Technology, GibcoBRL, Life Technologies), 4 μL BP buffer, and BP Clonase enzyme mix (Life Technologies) in 20 μL. Overnight incubation at 25° C. was followed by proteinase K treatment in the presence of 0.2% SDS and 10 mM EDTA at 45° C. for 15 min. We added 1 μg glycogen and extracted the reaction by using phenol/chloroform and chloroform; the sample was precipitated by using isopropanol. The precipitate was mixed with 300 ng pDEST12.2 (Life Technologies), 4 μL LR buffer, and 4 μL LR Clonase enzyme mix in a volume of 20 μL. The sample was further purified with proteinase K/phenol chloroform extraction followed by ethanol precipitation.

V) “Amplified Indirect” Protocol

The sample was treated as in the previous protocol (Gateway mediated bulk excision-“indirect”) until the BP Clonase reaction. We electroporated 1 μL of the 20-μL reaction into DH10B cells. The cells were spread on LB containing kanamycin, and the resulting colonies underwent plasmid extraction (Sambrook et al., 1989). The prepared plasmids were each reacted with LR Clonase and purified and then electroporated as before.

VI) “One-Tube” (“Direct”) Protocol

The procedure was the same as that for the indirect protocol until the BP Clonase reaction (Life Technologies). Then, we added 450 ng pDEST12.2, 6 μL LR Clonase enzyme mix, and 1 μL 0.75 M NaCl to the tube (total volume, 30 μL). The sample was treated with LR Clonase and purified as described. The BP/LR-reacted samples were dissolved in sterile water and electroporated into DH10B cells. The transformed cells were spread on LB plates containing either ampicillin or kanamycin and cultured overnight at 37° C.

To assess the conversion frequency of each excision method, we prepared the plasmids from 60 random colonies from LB plates. The plasmids were cut with PvuII, and the sizes of the inserts were analyzed by using 0.8% agarose gels. We also could assess the conversion efficiency by counting the colonies that grew on ampicillin- or kanamycin-containing plates.

### Example 13

### Homing Endonuclease System: A Vector For Ligation-Mediated Transfer of Inserts: λ-FLC-III-F

1) Insert cDNA Preparation

cDNA libraries were prepared by cloning the cDNA (prepared as in Carninci et al., 2000, Genome Research, 10:1617-1630) into the λ-FLC-III-F vector (Example 6), which carries the homing endonucleases I-CeuI and PI-SceI (New England Biolabs) at either side of the cloning sites (SalI and BamHI). These homing endonucleases, which recognize and cleave sequences of 26 and 39 bp respectively, do not cleave mouse genome (in fact, these homing endonucleases statistically cut once every 1.8×10¹⁸ base pairs and once every 1.2×10²⁴, respectively and therefore are very unlikely to cut even once high complex genomes such as Human and Mouse, whose total size is about 3×10⁹ base pairs). Therefore, they are optimal for subcloning cDNAs without internal cleavage of any of the tens of thousand clones in a library.

A phage cDNA library was prepared according to one variant of the cap-trapper technology (Carninci et al., 2000, Genome Research, 10:1617-1630) and cloned into λ FLC-III-F and amplified in C600 cells (Sambrook et al., 1989). We isolated the library phage DNA from 1 ml of the amplified phage solution by using the Wizard Lambda Preps DNA Purification System (Promega). Purified library phage DNA was digested with restriction enzymes (I-CeuI, PI-Sce I). First restriction step: a solution of 5 μl of 10×I-Ceu I buffer, 5 μl of 10×BSA and 2.5 U of I-Ceu I (total volume 50 μl) was prepared in a tube and incubated for 4 hour at 37° C.

After this step, the restriction tube was heated for 15 min at 65° C. The digested DNA was purified by proteinase K treatment (Sambrook et al., 1989), extracted with phenol/chloroform, and chloroform, and precipitated with isopropanol, and very carefully resuspended. The second step restriction was carried out as follows: redissolve the DNA in 40 μl of water, add 5 μl of 10×PI-Sce I buffer and, 4 U PI-Sce I (New England Biolabs, total volume 50 μl),and incubate for 4 h at 37° C. After this step, the restriction tube was heated for 15 min at 65° C. The digested DNA was purified by proteinase K treatment, extracted with phenol/chloroform, and chloroform, and precipitated with isopropanol, and very careful resuspension. (as in Sambrook et al., 1989).

2) pFLCM-f Preparation

λ-FLCIII-F vector (Example 6) was excised with in vitro cre-lox mediated recombinase. At first, 100 ng of λ-FLCIII-F were treated with 1 U cre-recombinase at 37° C. for 1 hour in 300 μl final volume. Then, extracted with phenol/chloroform, and chloroform, and precipitated with isopropanol (Sambrook et al., 1989). The plasmids were electroporatetd into E. coli DH10B (Life Technologies) at 2.5 kv/cm following the instruction of the manufacturer. Cells were spread on LB-agar (Sambrook et al., 1989) containing 50 μg/ml of ampicillin. To the surface of the agarose in the 9 cm petri dish, we added also 40 microliters of 2% X-gal and 7 microliters of 200 mM IPTG for colorimetric detection of the plasmid carrying the LacZ stuffer I to facilitate later identification of the background (for a theoretical consideration: Sambrook et al., 1989). The plate was cultured overnight at 37° C. and the day later several dozens colonies appear. We picked one blue colony from the above LB, inoculated in 50 ml of LB-broth/50 microgram/ml ampicillin and let grow overnight with 300 rpm shaking (Sambrook et al., 1989). Next day we prepared plasmid DNA by QIAprep spin mini prep kit (QIAGEN).

3) Plasmid Vector Preparation (Removal of the Stuffer I) (See Also FIG. 8)

This step is to prepare a plasmid (in this case pFLC-III-f) devoid of the stuffer I (in this case stuffer of FIG. 1f) to maximize the recombination.

Three μg of plasmids cDNA were digested with restriction enzymes (I-Ceu I, PI-Sce I ). In the first step restriction was done in total volume 50 μl in presence of 5 μl of 10×I-Ceu I buffer, (New England Biolabs), 5 μl of 10×BSA (bovine serum albumine supplied by New England Biolabs with the enzyme) and 4 U of I-Ceu I (New England Biolabs, and incubation for 4 hour at 37° C. After this step, the restriction tube was heated for 15 min at 65° C. Digested DNA was purified by proteinase K treatment, extracted with phenol/chloroform, and chloroform, and precipitated with isopropanol, and very carefully resuspended (Sambrook et al., 1989). The second restriction step was done in a total volume of 50 μl supplemented with. 5 μl of 10×PI-Sce I buffer (New England Biolabs), 4 U PI-Sce I (New England Biolabs,), and incubated for 4 hour at 37° C. After this step, the restriction tube was heated for 15 min at 65° C. Digested DNA was purified by proteinase K treatment, extracted with phenol/chloroform, and chloroform, and precipitated with isopropanol (Sambrook et al., 1989). After very careful resuspension, the digested DNA was separated in 0.8% low melting agarose gel (seaplaque agarose FMC) buffered with TAE (Tris-acetate-EDTA; see Sambrook et al., 1989). In the following step: after electrophoresis for 1.5 h at 50V, the DNA fragment corresponding to the empty plasmid vector (2.9 kb) was cut off from gel and purified by QIAGEN QIAquick Gel Extraction kit (QIAGEN).

4) Ligation of Cleveaged Plasmid pFLC-III-f and cDNA Insert (See Also FIG. 8)

7.5 ng of prepared insert and 100 ng of pFLCIII-f plasmid vector, prepared in the above step 3), were mixed in a final volume of 100 μl, containing also 10×T4 DNA ligase buffer (New England Biolabs) and DNA 200 U of T4 ligase (New England Biolabs) and incubated at 16° C. overnight. Ligated palasmids were electroporated into DH10B at 2.5 Kv(Kilovolt)/cm (Invitrogen) following the manufacturer's instruction. Cell were spread on LB containing ampicillin (as above), and cultured overnight at 37° C. We picked then randomly 12 colonies and prepared plasmids (inoculation in 3 ml LB-broth/50 microgram/ml ampicillin and let grow overnight with 300 rpm shaking (Sambrook et al., 1989). Plasmid DNA was prepared with a Quiagen plasmid DNA extraction kit.

The plasmids were cut with PvuII (New England Biolabs) in presence of 1×Pvu II buffer) and their insert size was analyzed using 0.8% TBE agarose gel stained with Ethidiumbromide (Sambrook et al., 1989).

5) Result

Titer: pFLCIII-f+ insert (cDNA):2.1×10⁴ pfu/ml

Insert size check (average size)

Excision protocol here presented: 3.07 kb

In vitro Cre-lox mediated recombinase (control experiment): 3.1 kb. The control experiment consisted in the same library excised with the Cre-lox following protocol as the example 12, (number III, in vitro Cre-lox mediated excision).

It has been known in the art that the use of restriction enzymes give high size bias. In fact, usually plasmid libraries prepared by ligation show half the size of lambda-excised cDNA libraries (in Table 2 the cerebellum library is 1.4 Kb in pBluescript while 3.36 Kb with λ-FLC-I-B: the size is only 41.6%, and therefore not very efficient).

In the current example, instead, the size with the homing nucleases is 3.07 kb versus 3.0 kb, the 99%, which is almost not relevant size bias (a 1% size bias enters in the statistical variability). In conclusion, we proved that the excision system using homing endonucleases restriction enzymes is an efficient excision system.

### Example 14

### Vectors For Size Selection and Background-Reducing Systems

The λ-FLC-I-B and other vectors shown in the FIGS. 1 and 2 has been used to successfully prepare libraries of full-length mouse cDNA, and showed to having a cloning capacity of ˜0.2 to 15.4 kb cDNAs.

When we tried to clone strongly subtracted cap-trapped cDNAs (according to the method described in Carninci et al., 2000, Genome Res., 10: 1617-1630), we found that because of the paucity of cDNA (less than 10 ng), using λ-FLC-I-B led to a certain background. When this background exceeded 20% to 30%, it affected the cost-performance of subsequent large-scale sequencing operations. To develop a vector associated with less background, we prepared a new, very effective method to decrease the background of λ-phage libraries that are excised into plasmids. We substituted the stuffer I in λ-FLC-I-B with that in FIG. 1e to produce the λ-FLC-I-E. The stuffer of this vector carries 2 copies of the “suicide gene” ccdB (Bernard and Couturier, 1992, J. Mol. Biol., 226: 735-745) and a functional LacZ for blue-white selection (FIG. 1f). Notice that the LacZ present in the pBluescript-derived fragment is nonfunctional because it is disrupted by either stuffer I or the cloned cDNA. Interestingly, λ phages carrying the cob gene can replicate in E. coli C600; this suggests that during the lytic cycle of the λ phage, DNA gyrase, the target of the ccdB gene product, is dispensable.

After the excision procedure, we plated the equivalent of up to 300 pg of the excised vector (without insert) but did not obtain any colonies. On the contrary, in a control experiment, we obtained more than 1175 colonies (equivalent to the background) when we plated the equivalent of ˜3.5 pg of a similar construct containing a 3.6-kb insert but without ccdB instead of the stuffer. This difference constitutes an impressive background reduction of at least 10⁵-fold, similar to that of λ-FLC-III-F (described later).

### Example 15

### DNA Contamination Background

All of the tested background-reducing stuffers like those in FIGS. 1d-f yielded undetectable background derived from nonrecombinant vectors and therefore can be considered interchangeable. With the vectors λ-FLC-I-E, λ-FLC-III-F, λ-FLC-III-D, λ-FLC-III-S-F, and λ-FLC-I-L-D, the background depend on the environmental DNA contamination. In a test experiment, we did not ligate any cDNA to λ-FLC-I-E. Because there was no background to reduce at the λ-plating stage, we obtained 8.4×10⁴ pfu/μg vector, which included the contribution of non recombinant vector, compared with typical values of >10⁷ pfu/μg for positive controls. We amplified the background plaques, excised the plasmids, analysed 12 clones, and sequenced representative samples showing different electrophoretic patterns. The background clones that remained after the selection were derived only from the E. coli genome, which was probably a residual from the dead E. coli cells during the vector DNA preparation, whereas no vector sequence was found in any insert. Therefore, if a goal is the complete absence of background, all contaminating genomic DNA must be eliminated from the λDNA preparations and, perhaps more importantly, cDNAs must have intact ends so that they are easily clonable.

### Example 16

### Background-Reduction loxP System

The background reduction associated with stuffer I differs from that of the stuffer in λ-FLC-I-E, because we independently tested a double strategy using a single copy of ccdB and an additional lox P site inserted into the stuffer I (FIG. 1f). During the excision process, the third lox P site favours the separation of the origin of replication from bla (the gene for β-lactamase, for conferring resistance to ampicillin), as shown in FIG. 1i. To eliminate this problem, we manipulated the order of the plasmid sequence and lox P elements in the λ-vector so that the lox P on stuffer I was between bla and the origin of replication. Neither of the defective excised plasmids can replicate or confer antibiotic resistance (FIG. 1i).

In a preliminary experiment, we constructed a λ-FLC-III-type vector that contained as a stuffer only the background-reducing sequence of FIG. 1i but without the ccdB gene. We obtained 43 colonies from ˜3.5 pg of the excised plasmid compared with 771 from ˜3.5 pg of a control excised plasmid of the same size that lacks both the lox P background reducing sequence and the ccdB gene. Therefore, the lox P background-reducing sequence eliminated 94.4% of the background. When ccdB was added to the lox P-containing stuffer, the resulting vector did not yield any colonies even when we electroporated up to 350 pg of excised plasmid, which had a background-reducing element like that in FIG. 1f. This result corresponds to a background reduction of at least 7.7×10⁴-fold, a factor similar to that obtained with the background-reducing element of the λ-FLC-I-E vector. The background-reducing systems of both the λ-FLC-III-F and λ-FLC-I-E vectors were considered sufficient for our full-length cDNA cloning purpose.

### Example 17

### Bulk Excision of cDNA Libraries

Before bulk excision, cDNA libraries are optionally amplified on a solid-phase medium according to the standard procedure (Sambrook et al., 1989).

This process does not decrease the size of the cDNA library, but because of the preferential packaging of long phages, decreases (but does not eliminate) the frequency of the phages that carry cDNA inserts of approximately ≦0.5 kb. Amplification in C600 cells eliminates hemimethylation, which is used to clone the cDNA (Carninci and Hayashizaki, 1999, as above). Hemimethylated cDNA of a primary cDNA library would be cleaved during the in vivo excision in BNN132 (described later).

I) Cre-Lox-Based Excision—In Vivo Solid-Phase Excision

The in vivo solid-phase excision process (representing the state of the art) seems straightforward (FIG. 3), simply requiring infection of the amplified cDNA library into the BNN132 bacterial strain, which constitutively expresses Cre-recombinase (Elledge et al., 1991, Proc. Natl. Acad. Sci. USA, 88:1731-5). However, this practice is not recommended, because of plasmid instability (Summers et al., 1984, as above) and low plasmid yield (Palazzolo et al., 1990, as above). In fact, Cre-recombinase is expressed constitutively, causing formation of plasmid dimers and multimers and leading to a high proportion of plasmid-free cells (Summers et al., 1984, as above), thereby impairing the sequencing efficiency. We confirmed that low plasmid yield and plasmid loss after prolonged culture are the rule when using BNN132 as a host strain for cDNA libraries.

II) Cre-Lox-Based Excision—In Vivo Liquid-Phase Excision

The in vivo liquid-phase excision process overcomes this problem of plasmid loss and poor yield after prolonged culture: we extracted the excised plasmid cDNA library after a brief culture at 30° C. or 37° C. and electroporate into any convenient E. coli strain, such as DH10B. Similar results in terms of size of the excised library were obtained after culture/excision for 1, 2, or 3 h at either 30° C., which is supposed to preserve the size of the library unbiased by keeping the plasmid at a low copy number (Lin-Chao et al., 1992, Mol. Microbiol., 6:3385-3393), or 37° C., at which plasmids are expressed at increased copy number. The copy number is also inversely proportional to the size of the cDNA inserts. When we excised a cDNA library cloned in λ-FLC-I-B, the final titer after the excision was 2.4×10⁸ cfu/μg after culture for 1 h at 30° C., 9.1×10⁸ cfu/μg after 2 h at 30° C., and 1.4×10⁹ cfu/μg after 3 h at 30° C. The titers after growth at 37° C. were 1.5×10⁹ cfu/μg after incubation for 1 h, 9.8×10⁸ cfu/μg after 2 h, and 2.8>10⁹ cfu/μg after 3 h. The average insert size was 4.1, 3.9, and 3.3 kb for 1, 2, and 3 h at 30° C., and 2.9, 3.6, and 3.8 kb for 1, 2, and 3 h at 37° C., respectively. These results suggested that there were no noteworthy excision-associated problems related to the length of inserts or to the temperature and duration of the BNN132 E. coli culture.

To better quantify the size bias associated with the Cre-lox excision system, we mixed an equal number of non-recombinant λ-FLC-I-B vectors carrying the 10-kb stuffer with phages from the amplified cDNA library, then infected the cells. The ratio of clones containing the 10-kb insert was close 50% at all of the described conditions. This result confirms the robustness against size bias of the Cre-lox excision system. Among the advantages of this in vivo liquid-phase excision method is the high DNA yield, which facilitates downstream operations, such as the production of consistent quantities of single-stranded plasmid DNA by using GeneII-ExoIII, which can be used for further normalization/subtraction of existing cDNA libraries (Bonaldo et al., 1996, Genome Res., 6:791-806) while avoiding plasmid amplification steps that could decrease the size of the amplified library.

III) Cre-Lox-Based Excision—In Vitro Excision

Although it does not show size bias, the in vivo liquid-phase excision procedure still involves a brief round of library amplification, which might cause sequence-specific representational bias. Therefore, we developed the in vitro excision method, which is based on Cre-mediated recombination.

This excision system uses purified λDNA from the amplified cDNA library, followed by electroporation. For this application, we tested the electroporation conditions described for long BAC inserts (Sheng et al., 1995, Nucl. Acids Res., 23:1990-1996). In light of our results from sizing 60 plasmids after restriction with PvuII, we did not find significant differences in the final size of the plasmid cDNA library when we used pulses between 1.7 and 2.5 kV/cm. We regard the Cre-lox in vitro excision protocol as the most suitable of those we tested, because it does not require even a brief amplification step of cDNA libraries in BNN132, is robust in terms of size bias, and can be used with all of the vectors described here.

IV) Gateway™-System-Mediate Excision

For λ-FLC-II-C, in addition to the Cre-lox excision protocol for excising a pFLC-II plasmid (FIG. 2h), we have developed protocols for bulk excision which are based on the Gateway system.

Inserts are at first transferred into an entry vector, the pDONR201 (Life Technologies), followed by transferring to a destination vectors, the pDEST12.2 (Life Technologies, structure not shown).

λ-FLC-II-C vector that we prepared carries the Gateway attB1 and attB2 sequences for transferring individual clones (Walhout et al., 2000, as above) or bulk libraries into different functional vectors (FIG. 2c) or into pFLC-DEST (FIG. 2j) for sequencing.

The three Gateway excision protocols (the “indirect”, “amplified indirect”, and “direct” protocols) are outlined in FIG. 3 and described above in the experimental part.

Any of the Gateway-mediated bulk-excision protocols was a valid alternative to the Cre-lox bulk excision procedure. In fact, the average size of 60 clones from the excised cDNA sublibraries was 2.3 kb for the control Cre-lox reaction (in vitro Cre-recombinase protocol), 2.4 kb with the “indirect” protocol, 2.5 kb with the “amplified indirect” protocol, and 3.3 kb with the “direct” protocol. The average size of this cDNA before excision was 3.7 Kb. Considering the final size close to the average size of mRNAs on gel, we considered the excision systems satisfactory. The Gateway-mediated excision system is anyway very attractive when sufficient cDNA is available for cloning into λ-FLC-II-C, which accommodates the use of the Gateway excision protocols. In light of the requirements of our sequencing operation, we used pFLC-DEST (FIG. 2j) as our destination vector.

### Example 18

### Comparative Example Between 6.0 kb and 5.5 kb Stuffer II Vectors

1) Vectors Construction

λ-FLC-I with 5.5 Kb stufferII was constructed as described before in the examples above. To compare the cloning size, λ-FLC-I with 6.0 Kb stufferII was constructed. We added a 0.5 Kb fragment in the HindIII site on the 5.5 Kb stufferII. 0.5 Kb fragment was obtained by restriction digestion with HindIII of mouse genomic DNA. Mouse genomic DNA was digested with HindIII and 0.5 Kb fragment was separated by gel electrophoresis. The fragment was subcloned into the pBluescript+ (stratagene) and cleaved by HindIII and inserted into HindIII site on the 5.5 Kb stufferII fragment subcloned into the pBluescript. The 6.0 Kb stufferII was recovered by the restriction digestion of AscI and ligated into λ left arm and right arm with 10 Kb stufferI and pBluescript.

2) Preparation of Arms For Cloning

λ-DNA was prepared by QIAGEN lambda Midi kit (#12543).

The cohesive termini of 10 μg of the lambda DNA were annealed by incubation for 2 hours at 42° C. in 180 μl of 10 mM Tris-Cl pH 7.5, 10 mM MgCl₂, and we added 20 μl of 10×Ligation buffer and 400 unit of T4 Ligase (both of NEB Kit), and incubated for 7 hours at room temperature, followed by ligase inactivated for 15 min at 65° C. The above λ-DNA was digested with restriction enzymes (all purchased from New England Biolabs, Inc.) in 3 steps by addition of 50 mM, 100 mM and then 150 mM NaCl (final concentration at each of the three steps). The first step restriction was done in 50 mM NaCl by addition of 2 μl of 5M NaCl, 10 μl of NEB 2 buffer, 73 μl of H₂O, 40 units of XhoI, 20 units of SpeI and 32 units of PacI for both vectors and then the sample was incubation for 2 hours at 37° C. The second step was done in 100 mM NaCl by addition of 2 μl of 5M NaCl, 20 μl of 10×NEB 3 buffer, 180 μd of H₂O and 20 units of SwaI and incubation for 2 hours at room temperature. After this step the reaction tube was heated for 15 min at 65° C. Finally, the third step was done in 150 mM NaCl by addition of 5 μl of 5M NaCl, 60 units of SalI and 60 units of BamHI, and incubation for 4 hours at 37° C. After restriction the DNA was purified by Proteinase K treatment in presence of 0.1% SDS and 20 mM EDTA, extracted with phenol/chloroform and chloroform, and precipitated with ethanol (Sambrook, et al., 1989). DNA concentration should not exceed 20 μg/ml to avoid resuspension problems. After very careful resuspension for at least 30 min the digested DNA was separated in 0.7% low-melting agarose gel (Seaplaque, FMC) in the followings steps. After electrophoresis for 1.5 hours at 8 V/cm the DNA fragments which was shorter than 19 Kb of the StyI-digested λ DNA were cut off from the gel (step 1). Then, the electrophoresis buffer (1×TBE) was changed for fresh one and the remained DNA in the gel were electrophoresed to the opposite orientation at 8 V/cm for 2.5 hours. At this point the shorter DNA than 19 kb were cut off again (step2). The buffer was changed again. The remainder of DNA in the gel were electrophoresed to the same orientation of the step 1 at 8 V/cm for 30 min in order to compact the region containing the λ arms DNA for shorter reaction volumes. Finally the λ arms DNA were cut off (step 3), and purified and checked as previously described (Carninci and Hayashizaki, 1999, as above) with β-agarase (NEB) after equilibration of the gel with TE buffer (Sambrook et al., 1989).

3) Construction of the Test Insert

250 bp Test Insert

λ-DNA was digested with PstI and electrophoresed in the 2% low melting agarose gel. 200-300 bp bands were cut off and purified by QIAquick Gel Extraction Kit (Qiagen). 200-300 bp PstI fragments were subcloned into the pBluescript and digested with BamHI and SalI. 250 bp BamHI-SalI fragmet was separated in 2.0% low-melting agarose gel and cut off and purified by Qiagen Kit.

2 kb Test Insert

The plasmid containing 2.0 Kb mouse cDNA was used as PCR template. 2 Kb insert was amplified with the 1stBS primer and 2ndXprimer and purified by Proteinase K treatment in presence of 0.1% SDS and 20 mM EDTA, extracted with phenol/chloroform and chloroform and precipitated with ethanol (Sanbrook, et al., 1989, as above). PCR products were digested with BamHI and XhoI (cohesive ends with SalI) and purified as described above.

6 Kb Test Insert

6 Kb test insert was prepared as described above for the previous inserts.

10 Kb Test Insert

p-FLC-I with 10 Kb stufferi was digested with BamHI and SalI and purified by proteinase K as described above. The 10 Kb BamHI-SalI fragment was separated with 0.7% low-melting agarose gel electrophoresis and isolated from gel with β-agarase (NEB) after equilibration of the gel with TE buffer (Sambrook et al., 1989)

4) Insert Size Check

4 kinds of test insert was ligated into μ-FLC-I with 5.5 Kb stufferIl and μ-FLC-I with 6.0 Kb stufferll. 200 bp, 2 Kb, 6 Kb and 10 Kb test inserts were ligated at ratio 1:1:1:1 or 3:1:1:1 to the both vectors, respectively.

Subsequently, the packaging reaction was performed using MaxPlax Lambda Packaging Extract (Epicentre Technologies). The phage solutions were amplified in C600 cells. 1×10⁴ pfu were plated on 90 mm dishes of LB-agar and topped with LB-agar containing 10 mM MgSO₄ and let grow overnight to confluence (Sambrook et al., 1989). The phages particles were eluted with SM-buffer and titered. The phage DNA was extracted and converted to plasmid with 1 U Cre-recombinase at 37° C. for 1 hour in 300 uL as recommended (Novagen, Madison, Wis., USA), and the purified by S400 spun column (Pharmacia). The excised plasmids were electroporated into DH10B cells (Life Technologies) at 2.5 KV/cm and plated on the LB-agar plate containing 100 ug/ml ampicillin. Each 96 colonies were picked up and the plasmid preparation was performed by the plasmid extraction automatic instrument, solutions and protocols obtained by KURABO (however, any other method of purification of plasmid, for instance according to Sambrook et al., 1989, can be used). The plasmids were digested with PvuII and insert size was checked by agarose gel electrophoresis.

Results are shown in Table 1.

TABLE 1

| | 5.5 kb stuffer II | 6.0 kb stuffer II |
|---|---|---|
| 10.0 kb insert | 5 | 3 |
| 6.0 kb insert | 43 | 27 |
| 2.0 kb insert | 42 | 50 |
| 0.25 kb insert | 3 | 2 |

Vectors stuffer II of 5.5 kb were able in 43 cases to accept inserts of 6 kb and in 5 cases inserts of 10 kb. The inserts of 6 and 10 kb corresponding to long and full-length cDNAs.

The result demonstrated that vectors comprising a stuffer II of 5.5 kb, allowed the insertion of cDNA inserts of long sizes (6.0 and 10.0 kb) more efficiently than vectors comprising a stuffer II of 6.0 kb. A vector having CS of 31.5 kb (that is stuffer II of 5.5 kb) is advantageous for preparing full-length cDNAs libraries than a vector having the CS size of 30 kb (that is stuffer II of 6 kb).

### Example 19

### The Gene Discovery is Correlated With the Average Insert Size of the cDNA Library

I) A Vector for Cloning Size-Selected cDNA With Ligation-Mediated Clone Transfer. λ-FLC-III-L-D (FIG. 2e)

Similar to λ-FLC-I-L-B and λ-FLC-I-L-D, λ-FLC-III-L-D lacks stuffer II and therefore is used for cDNA libraries with large inserts. This vector carries the same background-reducing element as λ-FLC-I-L-D, but λ-FLC-III-L-D differs from λ-FLC-I-L-D in that excision of λ-FLC-III-L-D yields a pFLCIII-d plasmid (the plasmid of FIG. 2i comprising the stuffer I of FIG. 1d), which is suitable for subcloning without internal cleavage of cDNAs.

II) A Vector for Short cDNAs and Ligation-Mediated Transfer of Inserts: λ-FLC-III-S-F (FIG. 2f)

The mRNA of many organisms that are evolutionarily far from vertebrates, such as Arabidopsis thaliana and Oryza sativa (rice), is shorter (typically 1 to 1.5 kb on an agarose gel) than that of vertebrates. When working with invertebrates, size selection like that used in all of the previously described examples may bias for long inserts, which may not be representative of the starting mRNA. Even though gene discovery from 3 rice libraries has been excellent even when we use λ-FLC-I-B, we prepared λ-FLC-III-S-F to address this concern. λ-FLC-III-S-F is the same as the previously described λ-FLC-III-F but has a longer stuffer 11 (6.3 kb). With the 6.3-kb stuffer II, the nominal cloning size is 0 to 14.9 kb, which facilitates cloning relatively short cDNAs. The background-reducing element of λ-FLC-III-S-F is that in FIG. 1f, and this vector produces, after excision, a pFLCIII-f plasmid (the plasmid of FIG. 2i comprising the stuffer I of FIG. 1f).

III) Full-Length cDNAs

The full-length cDNA we used was prepared as described (Carninci and Hayashizaki, 1999, as above) and was normalized/subtracted (Carninci et al., 2000, Genome Res., 10:1617-1630). cDNA prepared with any other technique can be directionally cloned into the λ-FLC vectors, provided that the restriction sites are compatible or that the vector is properly modified.

The average insert size of cDNA cloned into λ-FLC-I-B was always longer than that for the same cDNA cloned into other vectors (Table 2; average size of cDNA libraries using various vectors).

TABLE 2

| Tissue | Vector | titer | size (Kbp) |
|---|---|---|---|
| Placenta | λ-ZAP II | 4.6 × 10⁵ | 1.3 |
| Placenta | λ-FLC-I-B | 1.8 × 10⁵ | 2.34 |
| Cerebellum | pBluescript | 8.6 × 10⁴ | 1.4 |
| Cerebellum | λ-FLC-I-B | 3.7 × 10⁵ | 3.36 |

The average insert size of the λ-FLC-I-B library was 1.8 times larger than that of the λ-ZapII library and 2.4 times larger than that of the plasmid cDNA library.

We correlated the average insert size of each cDNA library in Table 3 and FIG. 4 with the complexity of the library. In fact, these libraries were sequenced for the gene discovery program during the construction of the full-length cDNA encyclopedia (RIKEN mouse cDNA encyclopedia, RIKEN and Fantom Consortium, Nature, Vol. 409: 685-690. The redundancy obtained by sequencing randomly picked clones and clustering clones with the same ends (Konno et al., 2001, as above) was compared by using 7 cDNA libraries cloned in λ-Zap II (conventional vector) and 9 cDNA libraries cloned in λ-FLC-I-B (Table 3). To facilitate comparing differences in the complexity of these libraries, we show not only the clustering data after completion of sequencing of a given library but also the number of clusters after the available number of runs closest to 5000 sequencing passes. The conventional vector did not accommodate the preparation of complex, low-redundancy cDNA libraries from any tissue. In contrast, all of the normalized/subtracted cDNA libraries cloned into λ-FCL-I-B showed higher complexity (average, 3392 clusters/4826 reactions; redundancy, 1.42) than did normalized/subtracted libraries with the conventional vector (average, 2089 clusters/4773 reactions; redundancy, 2.28). Even if we cannot expect to know a priori the variety (or complexity) of gene expression in a given organ, the complexity was supposed to be very high for the pooled total “embryo 10+11” library (Table 3). However, the “embryo 13 forelimb” library, which is cloned in λ-FCL-I-B and which covers a relatively restricted biological phenomenon, showed higher complexity than did the “embryo 10+11” library, which surely contains an increased variety of genes because it includes many developing organs and neuronal tissues.

A more direct comparison comes from the libraries made from embryonic stem cells (ES cells); these libraries were all prepared from the same starting RNA. The number of clusters after 5104 sequencing reactions (total number of sequenced samples) is 3068 for the λ-FCL-I-B-cloned cDNA but just 2362 after 5160 sequencing reactions for the library in the conventional vector. That is, 31% more clusters were discovered by using λ-FCL-I-B. The difference is even more striking after additional sequencing reactions : 4971 clusters were categorized after 10514 sequencing reactions for the λ-FCL-I-B-based library and only 3795 clusters after 10492 sequencing reactions of the conventional ZAP vector library (see FIG. 14); then, 15 520 sequencing passes of the conventional ZAP vector library (48% more) led to only 4566 clusters (9% fewer)) FIG. 14). Notice also that although both the ES cell libraries were normalized and mildly subtracted with the same drivers, the C3 library (which was in λ-FCL-I-B) was also subtracted with genes that were already categorized. Although we expected that a strongly subtracted library would contain a lower variety of genes, this was not the case.

These data support the notion that the capacity to clone long cDNAs accelerates new gene discovery when full-length approaches are used. In addition, the introduction of the λ-FCL vectors during the course of the preparation of the mouse cDNA encyclopedia restored a high rate of gene discovery (Table 3).

Noteworthy also is the increased rate of new genes identified by using 5λ-end readings of λ-FLC-based libraries, which suggested that previously available cloning protocols and vectors have biased the gene discovery for short cDNAs.

The λ-FLC vector family according to the invention demonstrated to be a powerful tool for high-efficiency cloning of full-length cDNA, gene discovery, and bulk transfer of selected cDNA clones into vectors for functional analysis, such as expression vectors.

### Example 20

### λ-BAC vector construction

1) Preparation of “Component 1” (FIG. 9)

10 μg of plasmid named pFLC-III-e were digested with 10 units of restriction enzyme BssHII (New England Biolabs also indicated as NEB) in 20 μl of 1× supplied buffer (NEB) at 37° C. for 1 hour. The pFLC-III-e/BssHII was separated with TAE (Tris-acetate-EDTA buffer, Sambrook et al., 1989) 0.8% low-melting agarose gel (SeaPlaque, FMC) at 50 V for 1 hour (see Sambrooket al, 1989). The plasmid band was cut out from the gel and digested with β-agarase (New England Biolabs) as suggested by the manufacturer (alternatively, also the standard technique described in Sambrook et al., 1989 can be used).

The 5 kb of stuffer I was cut out from the gel and sliced. The gel was mixed with 1 ml of 1× β-agarase buffer (NEB). The tube containing the gel was put on ice for 30 min to equilibrate with 1× β-agarase buffer. The buffer was removed from the tube by pipetting and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) were added to the tube and incubated for 5 hours. Phenol/chloroform extraction was done and precipitated with ethanol according to standard techniques (Sambrook et al., 1989). The precipitated 5 kb fragment was dissolved with 5 μl of TE (10 mM Tris-HCl, 1 mM EDTA, pH 7.5) and indicated as “component 1”.

2) Preparation of “component 2” (FIG. 9)

A pBeloBAC11 derivative prepared according to FIG. 1 of U.S. Pat. No. 5,874,259 (herein incorporated by reference) was used in the following “preparation of component 2” experiment. According to the description of U.S. Pat. No. 5,874,259, the basic pBeloBAC11 (Kim et al., 1996, Genomics, 34:213-218) was modified by as following: ligating together the oriV element (SEQ ID NO:43) and the FRT element (SEQ ID NO:44) and the resulting fragment was made blunt and ended and then ligated into the XhoI site which had been made blunt end. The orientation of the two joined fragments is such that when the fragment is cloned into the XhoI site, the ori is physically located between the nearby FRT site and the insert cloning site.

3 μg of this pBeloBAC11 derivative (FIG. 9) was cleaved with 10 U of the restriction enzyme SalI (NEB) in 30 μl as recommend by the manufacturer (37° C. in the supplied buffer) and then dephosphorylated by adding 1 unit of CIP (Calf Intestinal Phosphatase)(Takara, Japan) at 37° C. for 30 min (a general use of dephosphorylation to reduce the cloning background is disclosed in Sambrook et al., 1989) followed by separation using TAE 0.8% low-melting point agarose (SeaPlaque, FMC) at 50 V for 1 hour (standard technique, Sambrook et al., 1989).

The agarose gel region containing the plasmid fragment of 6.7 kb indicated in FIG. 9 as “component 2” was cut out of the gel (approximately 200 microliters) and digested with 10 units of β-agarase (NEB) for 5 hours, extract with phoenol/chloroform and then followed by ethanol precipitation same as shown in component 1.

3) Preparation of “Component 3” (FIG. 9)

A double strand oligonucleotide “adaptor” (FIG. 9) comprising the upper strand: 5′-pTCGAAGCTTCCG-3′ (SEQ ID NO:45) phosphorylated at the 5′ end and the lower strand: 5′-CGCGCGGAAGCT-3′ (SEQ ID NO:46) was prepared using oligosynthesized using an automated synthesizer (EXPEDITE 8909 using the standard protocol and reagents).

4) Ligation of “Components 1, 2 and 3” (FIG. 9)

“Component 1” (pFLC-III-e/BssHII fragment), “component 2” and “component 3” were mixed together in the ratio of 50 ng: 37 ng: 0.1 ng in the presence of 1÷ buffer (prepared by dilution to 1/10 from a stock of 10× supplied by the manufacturer NEB), 400 units of T4 DNA ligase (NEB) in final 5 μl of final volume reaction (buffer 1× dilution, DNA, adaptor, DNA ligase).

The mixture was incubated at 16° C. overnight to complete the ligation reaction.

After the addition of NaCl at 0.2 M final concentration into the ligation reaction, the ligation products were precipitated with 2 volumes of 96% ethanol and 1 μg of Glycogen (Roche)—according to the standard techniques (Sambrook et al., 1989) and the ligated products were recovered by ethanol precipitation according to standard protocol (Sambrook et al., 1989). The ligation products were dissolved in 10 μl of H₂O.

1 μl of the recovered ligation products were electropotrated into 20 μl of DH10B electrocomponent cells (Invitrogen) at 2.5 KV.cm (according to Invitrogen) instructions followed by plating the elctroporeted plasmid cells on LB-agar-supplemented with ampicillin at 50 μg/ml. To select positive clone which has modified pBAC, having the construct with the desired insert (“component 1”), randomly picked clones were cultured and plasmids checked (see Sambrook et al for general strategy of selecting and analyzying recombinants plasmids). A plasmid (modified pBAC of FIG. 9) having the stuffer I as indicated in FIG. 1e as insert is then selected for the next step

5) Introduction of loxP and XbaI Sites (FIG. 10)

In order to introduce loxP and XbaI sites into the modified pBAC prepared as above, 1 μg of the modified pBAC was mixed with 0.5 μM of “primer 1” (5′-AGAGAGAGAGATCTAGAATAACTTCGTATAATGTATGCTATACGAAGTTA TCTGTCAAACATGAGAATTG-3′)(SEQ ID NO:47), 0.5 μM of “primer 2”: (5′-GAGAGAGAGATCTAGATAACTTCGTATAGCATACATTATACGAAGTTATC GAATTTCTGCCATTCAT-3′)(SEQ ID NO:48), 125 μM dNTP mix, 1× “GC buffer 1” (Takara, Japan), 5 units of LA-Taq (Takara, Japan) in a volume of 50 μL.

Then, the following PCR amplification cycle was repeated for 25 times; step 1: 94° C. for 5 sec; step 2: 50° C. for 5 sec, 72° C. for 12 min.

After amplification, 1 μl of 0.5M EDTA, 1 μl of 10% SDS and 1 μl of proteinaseK, (10 mg/ml stock) (Sigma) were added to the PCR products obtained, incubated at 45° C. for 15 min and followed by phenol/chloroform treatment, chloroform extraction and then ethanol precipitation (Sambrook et al., 1989). After ethanol precipitation, the pellet was dissolved with water and cut with 15 units of restriction enzyme XbaI (NEB) in the buffer supplied by the manufacturer (NEB). PCR product was purified after electrophoretic separation with TAE 0.8% low-melting agarose gel (SeaPlaque, FMC) at 50 V for 1 hour (Sambrook et al., 1989). The PCR product was cut and digested with 10 units of beta-agarase (NEB) as suggested by the manufacturer (alternatively, also the standard technology disclosed in Sambrook et al., 1989 can be used).

The 11.7 kb of PCR product was cut out from the gel and sliced. The gel was mixed with 1 ml of 1× β-agarase buffer (NEB). The tube containing the gel was put on ice for 30 min to equibrate with 1× β-agarase buffer. The buffer was removed from the tube and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) were added to the tube and incubated for 5 hours. Phenol/chloroform extraction was done and precipitated with ethanol following standard techniques (Sambrook et al., 1989). The precipitated 11.7 kb fragment was dissolved with 5 μl of TE (10 mM Tris-HCl, 1 mM EDTA, pH 7.5) and indicated as “component 4” (FIG. 10).

### 6) Preparation of Stuffer II (“Component 5”)(FIG. 11)

To prepare the 1.8 kb stuffer as a size balancer (also indicated as “stuffer II”), 3 μg of mouse genomic DNA was digested with 20 units of Sau3AI and 1× supplied buffer (Nippon Gene, Japan) for 2 hours at 37° C. in a volume of 20 μl. The digested DNA was separated with 1.2% low-melting agarose gel at 50 V for 2 hours with lambda/Styl molecular marker (Nippon Gene, Japan). DNA fragments that migrated showing a size of about 1 1.8 kb were cut out of the gel and sliced. The gel was mixed with 1 ml of 1× β-agarase buffer (NEB). The tube containing the gel was put on ice for 30 min to equibrate with 1× β-agarase buffer. The buffer was removed from the tube and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) were added to the tube and incubated for 5 hours. Phenol/chloroform extraction was done and precipitated with ethanol following standard techniques (Sambrook et al., 1989). The precipitated 1.8 kb stuffer II DNA was dissolved with 10 μl of TE (10 mM Tris-HCl, 1 mM EDTA, pH 7.5).

The purified 1.8 kb DNAs (100 ng) was ligated with 10 ng Sau3AI/XbaI adaptor comprising the upper strand: 5′- GAGAGAGAGATCTAGAAAGCTCCA-3′ (SEQ ID NO:49), and the lower strand: 5′- GATCTGGAGCTT-3′ (SEQ ID NO:50) for 16 hours at 16° C. in the presence of 1× ligation buffer (diluted stock as above described) and 400 units of T4 DNA ligase (NEB) in a final volume of 5 μl. After inactivation of the ligase at 65° C. for 5 min, the ligation products were separated by TAE 1.2% low-melting agarose gel (SeaPlaque, FMC) at 50 V for 1 hour (Sambrook et al., 1989). again and 1.8 kb DNA was cut and digested with beta-agarase (NEB) as suggested by the manufacturer (alternatively, the technique described in Sambrook et al., 1989 can be used).

The 1.8 kb of PCR product was cut out from the gel and sliced. The gel was mixed with 1 ml of 1× β-agarase buffer (NEB). The tube containing the gel was put on ice for 30 min to equibrate with 1× β-agarase buffer. The buffer was removed from the tube and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) was added to the tube and incubated for 5 hours. Phenol/chloroform extraction was done and precipitated with ethanol following standard techniques (Sambrook et al., 1989). The precipitated 1.8 kb fragment was dissolved with 5 μl of TE (10 mM Tris-HCl, 1 mM EDTA, pH 7.5).

The 1.8 kb of the purified DNA was amplified using 0.5 μM XbaI primer (5′-GAGAGAGAGATCTAGAAAGCTCCA-3′)(SEQ ID NO:49), 125 μM dNTPs mix, 1× GC buffer I (Takara, Japan), 5 units of LA-Taq (Takara)in a final volume of 50 μl.

For the PCR amplification of DNA, the following cycle was repeated 25 times: step 1: 94° C. for 5 sec; step2: 68° C. for 1.5 min.

After amplification, 1 μl of 0.5M EDTA, 1 μl of 10% SDS and 1 μl of proteinaseK, (10 mg/ml stock) (Qiagen) were added to the PCR products obtained, incubated at 45° C. for 15 min and followed by phenol/chloroform treatment, chloroform extraction and then ethanol precipitation (Sambrook et al, 1989). After ethanol precipitation, the pellet was dissolved with water and cut with 15 units of restriction enzyme XbaI (NEB) in the buffer supplied by the manufacturer (NEB).

PCR products/Xbalwere separated with TAE 0.8% low melting point gel at 50V for 1 hour and cut out a 1.8 kb DNA fragment. This DNA fragment was digested with beta-agarase (NEB) as suggested by the manufacturer.

The 1.8 kb of PCR product was cut out the gel and sliced. The gel was mixed with 1 ml of 1× β-agarase buffer (NEB). The tube containing the gel was put on ice for 30 min to equibrate with 1× β-agarase buffer. The buffer was removed from the tube and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) were added to the tube and incubated for 5 hours. Phenol/chloroform-extraction was done and precipitated with ethanol following standard techniques (Sambrook et al., 1989). The precipitated 1.8 kb fragment was dissolved with 5 μl of TE (10 mM Tris-HCl, 1 mM EDTA, pH 7.5).

The purified PCR products/XbaI were named “component 5” (see FIG. 11).

7) Preparation of “Component 6” (FIG. 12)

The cohesive termini (cos ends) of 10 μg of the (linear) λ-FLC-I-E (FIG. 2a) annealed (the two complementary cos ends and the ends anneal to each other after this treatment; this increase ligation efficiency in later steps and simplify further procedures) by incubation for 2 hours at 42° C. in 180 μl of 10 mM Tris-Cl (pH 7.5), 10 mM MgCl₂, and 20 μl of 10× ligation buffer provided by NEB. 400 units of T4 DNA ligase (NEB) were added to the solution, and the sample was incubated for 5 hours at room temperature, followed by ligase inactivation for 15 min at 65° C. The k DNA with the cos-ends ligated in the previous step was digested with 5 units of XbaI (Nippon Gene, Japan), 1× manufacturers supplied buffer for 2 hours at 37° C. in a volume of 50 μl. After digestion, 1 μl of 0.5M EDTA, 1 μl of 10% SDS and 1 μl of proteinaseK, (10 mg/ml stock) (Qiagen) were added to the DNA obtained, incubated at 45° C. for 15 min and followed by phenol/chloroform treatment, chloroform extraction and then ethanol precipitation (Sambrook et al., 1989). After ethanol precipitation, the pellet was dissolved with water for 30 min while the tube was kept on ice, the digested DNA was separated in TAE 0.6% low-melting agarose gel at 50 V for 5 hours. Cos-ligated fragment (29 kbp) was cut out the gel and sliced. The gel was mixed with 1 ml of 1× β-agarase buffer (NEB). The tube containing the gel was put on ice for 30 min to equibrate with 1× β-agarase buffer. The buffer was removed from the tube and put a new 1× β-agarase buffer. The tube was put on ice for 30 min. This buffer exchange cycle was repeated once more. The buffer was removed and the tube was incubated at 65° C. for 5 min to melt the gel. 10 unit of β-agarase (NEB) were added to the tube and incubated for 5 hours. Phenol/chloroform extraction was done and precipitated with ethanol following standard techniques (Sambrook et al. 1989). The precipitated 29 kb cos-ligated fragment was dissolved with 5 μl of TE (10 mM Tris-HCl, 1 mM EDTA, pH 7.5), named “component 6” (FIG. 12).

8) Ligation of “Components 4, 5 and 6” (FIG. 12)

The “component 4” (modified pBAC), “component 5” (stuffer) and “component 6” (arms) were mixed in the following ratio: 120 ng: 19 ng: 300 ng, in presence of 1× ligation buffer (NEB ligation buffer) and 400 units of T4 DNA ligase NEB in 5 μl for 16 hours at 16° C.

After in vitro packaging (“MaxPlax™ Lambda Packaging Extract”, EPICENTRE TECHNOLOGIES,. Madison Wis., US) and plating the recombinant λ-phage (as described in Sambrook et al., 1989), a few hundreds plaques of λ phages were obtained.

5 clones (phage plaques) were randomly selected according to the method described in Sambrook et al., 1989.

The picked phage plaques were put in SM Buffer (Sambrook et al., 1989) and left at room temperature for 1 hour. Then, the eluted phage solution was used to infect C600 cells and were amplified according to the standard protocol (Sambrook et al., 1989).

In 3 out 5 clones we obtained the desired inserts (corresponding to “component 1”) by analysis with restriction enzymes (XbaI+BamHI+SalI, XbaI+BamHI, XbaI+SalI) (Sambrook et al., 1989). One of this clone, named λ-FLC-III-pBAC (FIG. 12) shown the same cloning range of other described λ-vectors (for example, λ-FLC-I-B, λ-FLC-II-C, λ-FLC-III-F) which was 0.2-15.4 kb.

TABLE 3
Lambda-Flcl allows preparing longer cDNA libraries, which is correlated to higher complexity
and higher gene discovery rate

| | | | | Clusters | | | Final | | | | 5′ |
|---|---|---|---|---|---|---|---|---|---|---|---|
| | | | | at fixed sequence (1) | | | extent of sequencing (2) | | | Coding | novelty |
| Code | Tissue | Titer | Size (Kbp) | sequences | clusters | redundancy | sequences | clusters | redundancy | (3) % | (4) % |

Conventional vectors (5)

| | | | | | | | | | | | |
|---|---|---|---|---|---|---|---|---|---|---|---|
| 6-100 | kidney | 3 × 10exp5 | 1.21 | 4680 | 1439 | 3.25 | | | | 99.1 | 6.5 |
| 22-100 | stomach | 3.5 × 10exp5 | 1.33 | 4447 | 1987 | 2.24 | | | | 82.1 | 12.4 |
| 22-104 | stomach | 2.0 × 10exp5 | 1.08 | 4068 | 1960 | 2.08 | | | | 82.1 | 6.38 |
| 23-100 | tongue | 4.1 × 10exp4 | 1.81 | 5016 | 2514 | 2 | 10295 | 4021 | 2.56 | 76.8 | 9.8 |
| 24-100 | ES cells | 1.3 × 10exp5 | 1.69 | 5160 | 2362 | 2.18 | 15520 | 4566 | 3.4 | 88.6 | 7 |
| 25-100 | embryo 13, liver | 8.5 × 10exp4 | 1.63 | 5005 | 1502 | 3.33 | 5864 | 1679 | 3.49 | 92.2 | 5.85 |
| 28-104 | total embryo 10 + 11 | 8.8 × 10exp5 | 1.8 | 5040 | 2859 | 1.76 | 9450 | 4470 | 2.11 | 93.9 | 5.69 |
| Average | | 2.8 × 10exp5 | 1.51 | 4773 | 2089 | 2.28 | 10,282 | 3681 | 2.79 | 87.8 | 7.66 |

Lambda Flc-l (6)

| | | | | | | | | | | | |
|---|---|---|---|---|---|---|---|---|---|---|---|
| 49-304 | testis | 2.6 × 10exp6 | 2.36 | 5000 | 3520 | 1.42 | 9015 | 5502 | 1.64 | 93.1 | 46.57 |
| 49-305 | testis | 8.9 × 10exp5 | 2.52 | 5120 | 3606 | 1.42 | 11564 | 6605 | 1.75 | 93.1 | 36.57 |
| 53-304 | pituitary gland | 2.1 × 10exp6 | 2.93 | 5073 | 3242 | 1.56 | 8059 | 4662 | 1.73 | 100 | 17.41 |
| 58-304 | thymus | 1.7 × 10exp6 | 3.81 | 5085 | 3742 | 1.36 | 10259 | 6445 | 1.59 | 80 | 21.6 |
| 59-304 | embryo 13, forelimb | 3.9 × 10exp6 | 3.19 | 3908 | 2865 | 1.36 | | | | 60 | 16.05 |
| 63-304 | medulla oblungata | 6.0 × 10exp5 | 2.89 | 4001 | 2998 | 1.33 | | | | 75 | 21.7 |
| 63-305 | medulla oblungata | 4.8 × 10exp5 | 2.97 | 5060 | 3654 | 1.38 | 8339 | 5358 | 1.56 | 75 | 29.7 |
| 64-305 | olfactory brain | 5.7 × 10exp5 | 3.01 | 5085 | 3835 | 1.33 | 10179 | 6394 | 1.59 | 80 | 23.9 |
| C3-300 | ES cells | 1.5 × 10exp5 | 2.45 | 5104 | 3068 | 1.66 | 10,514 | 4971 | 2.12 | 78.8 | 19 |
| Average | | 1.4 × 10exp6 | 2.9 | 4826 | 3392 | 1.42 | 9704 | 5705 | 1.71 | 81.6 | 25.8 |

(1) calculated by using a number of plates that give the value closest to 5000, for easy comparison of library complexity
(2) Some libraries were further sequenced
(3) Presence of the first ATG of annotated mouse genes
(4) Novelty of 5′ end ESTs versus databases
(5) Lambda ZAP II. cDNA size is shown after bulk excision of to plasmid library
(6) After in-vitro excision and electroporation into DH10B cells

1. A cloning bacteriophage vector comprising a construction segment (CS) and a replaceable segment (RS), wherein the size of CS is: X−1.2 kb≦CS<X; wherein X corresponding to the minimum size necessary to the vector for undergoing packaging.

2. The cloning vector of claim 1, wherein the size of CS is: X−0.2 kb.

3. A cloning bacteriophage vector comprising a construction segment (CS) and a replaceable segment (RS), wherein the size of CS is: 36.5 kb≦CS<38 kb.

4. The cloning vector of claim 3, wherein CS is 37.5 kb.

5. The cloning vector of claim 4, wherein CS is or comprises a foreign segment of 5.5 kb.

6. The cloning vector of claims 1-5, wherein said bacteriophage is λ.

7. The cloning vector of claim 1, wherein CS is a bacteriophage vector segment modified by comprising a plasmid segment at least comprising a ori.

8. The cloning vector of claim 7, wherein said plasmid segment comprising a ori is selected from the group of: pBluescript (+), pUC, pBR322, and pBAC.

9. The cloning vector of claim 1, wherein CS further comprises at least a selectable marker selected from the group consisting of: a DNA segment that encodes a product that provides resistance against otherwise toxic compounds; a DNA segment that encodes a product that suppresses the activity of a gene product; a DNA segment that encodes a product that is identifiable; a DNA segment that encodes a product that inhibits a cell function; a DNA segment that provides for the isolation of a desired molecule; a DNA segment that encodes a specific nucleotide recognition sequence which is recognized by an enzyme.

10. The cloning vector of claim 9, wherein said selectable marker comprises at least a marker selected from the group consisting of an antibiotic resistance gene, an auxotrophic marker, a toxic gene, a phenotypic marker, an enzyme cleavage site, a protein binding site; and a sequence complementary to a PCR primer sequence.

11. The cloning vector of claim 1, wherein said RS is flanked by two recombination sites, and said two recombination sites do not recombine with each other.

12. The cloning vector of claim 11, wherein said two recombination sites are selected from the group consisting of attB, attP, attL, attR and derivatives thereof.

13. The cloning vector of claim 11, wherein said two recombination sites flanking RS are lox recombination sites, which do not recombine with each other.

14. The cloning vector of claims claim 1, wherein CS further comprising two lox recombinant sites, said two lox recombination sites being capable of recombine with each other.

15. The cloning vector of claims 13-14, wherein the recombinant sites are loxP sites or derivatives thereof.

16. The cloning vector of claims claim 1, wherein RS further comprising at least a background-reducing sequence.

17. The cloning vector of claim 16, wherein said at least a background-reducing sequence is selected from the group consisting of: i) the ccdB gene, ii) the lacZ gene, iii) a lox sequence.

18. The cloning vector of claim 17, wherein said iii) lox sequence is loxP or a derivative thereof.

19. The cloning vector of claims claim 1, wherein RS is flanked by i) two homing endonuclease asymmetric recognition site sequences, which do not ligate with each other; or ii) two restriction asymmetric endonuclease cleavage sites sequences, which do not ligate with each other, recognizable by class IIS restriction enzymes.

20. The cloning vector of claim 19, wherein said homing endonuclease is selected from the group consisting of: I-CeuI, PI-SceI, PI-PspI, and I-SceI.

21. The cloning vector of claim 20, wherein said homing endonuclease asymmetric recognition site sequences are sequences from 18 to 39 bp.

22. The cloning vector of claims claim 1, which is linear.

23. The cloning vector of claim claim 1, wherein RS is replaced by a nucleic acid insert of interest.

24. The cloning vector of claim 23, wherein said insert is selected from the group consisting of DNA, cDNA and RNA/DNA hybrid.

25. The cloning vector of claim 23, wherein said insert is a long cDNA.

26. The cloning vector of claim 23, wherein said insert is a full-length cDNA.

27. The cloning vector of claim 26, wherein said full-length cDNA is a normalized and/or subtracted full-length cDNA.

28. A method for cloning a nucleic acid insert of interest or for preparing a bulk nucleic acid library of interest, comprising the steps of:
(a) preparing at least a cloning bacteriophage vector comprising a construction segment (CS) and a replaceable segment (RS), wherein the size of CS is: X−1.2 kb≦CS<X; wherein X corresponding to the minimum size necessary to the vector for undergoing packaging.
(b) replacing RS with a nucleic acid insert of interest into the cloning vector obtaining the product according to claim 23;
(c) allowing the in vivo or in vitro excision of the nucleic acid insert of interest or of the plasmid comprising the nucleic acid insert of interest;
(d) recovering the (recombinant) plasmid carrying the nucleic acid insert of interest or a library of these plasmids.

29. The method of claim 28, wherein between step b) and c) a step of amplification of the cloning vector is carried out.

30. A bacteriophage cloning vector comprising a construction segment (CS) and a replaceable segment (RS), wherein said RS comprises at least the ccdB gene.

31. A bacteriophage or plasmid cloning vector comprising a construction segment (CS) and a replaceable segment (RS), wherein said RS comprises at least a recombination site or a derivative thereof; or RS is flanked by two asymmetric site sequences, which do not ligate with each other, and are recognized by restriction endonucleases.

32. The cloning vector of claims 30-31, wherein said bacteriophage is λ.

33. The cloning vector of claim 30, wherein the size of the bacteriophage vector CS is: 32 kb≦CS≦45 kb.

34. The cloning vector of claim 30, wherein CS is: 36.5 kb≦CS<38 kb.

35. The cloning vector of claim 34, wherein CS is 37.5 kb.

36. The cloning vector of claim 31, wherein said recombination site is lox recombination site or a derivative thereof.

37. The cloning vector of claim 36, wherein said lox site is a loxP site or derivatives thereof.

38. The cloning vector of claim 30, wherein the CS of said vector comprises a plasmid segment at least comprising an ori.

39. The cloning vector of claim 38, wherein said plasmid segment comprising an ori is selected from the group consisting of :pBluescript(+), pUC, pBR322 and pBAC.

40. The cloning vector of claim 30, wherein CS further comprises at least a selectable marker selected from the group consisting of: a DNA segment that encodes a product that provides resistance against otherwise toxic compounds; a DNA segment that encodes a product that suppresses the activity of a gene product; a DNA segment that encodes a product that is identifiable; a DNA segment that encodes a product that inhibits a cell function; a DNA segment that provides for the isolation of a desired molecule; a DNA segment that encodes a specific nucleotide recognition sequence which is recognized by an enzyme.

41. The cloning vector of claim 40, wherein said selectable marker comprises at least a marker selected from the group consisting of an antibiotic resistance gene, an auxotrophic marker, a toxic gene, a phenotypic marker, an enzyme cleavage site, a protein binding site; and a sequence complementary to a PCR primer sequence.

42. The cloning vector of claim 30, wherein said RS is flanked by two recombination sites, and said recombination sites do not recombine with each other.

43. The cloning vector of claim 42, wherein said recombination sites are selected from the group consisting of attB, attP, attL, attR, and derivatives thereof.

44. The cloning vector of claim 42, wherein said two recombination sites flanking RS are lox recombination sites or derivatives thereof and do not recombine with each other.

45. The cloning vector of claim 44, wherein the lox recombination site is loxP or a derivative thereof.

46. The cloning vector of claim 30, wherein CS further comprising two recombinant sites or derivatives thereof, these two recombination sites being capable of recombine with each other.

47. The cloning vector of claim 46, wherein said two recombination sites are lox recombination sites or derivatives thereof.

48. The cloning vector of claim 47, wherein said lox recombination site is loxP or a derivative thereof.

49. The cloning vector of claim 30, wherein said RS further comprises the lacZ gene.

50. The cloning vector of claim 30, wherein said asymmetric site sequences are i) two homing endonuclease asymmetric site sequences or ii) two restriction endonuclease cleavage sites sequences recognizable by class IIS restriction enzymes.

51. The cloning vector of claim 50, wherein said restriction homing endonuclease capable of cutting said asymmetric site sequences is selected from the group consisting of: I-CeuI, PI-SceI, PI-PspI and I-SceI.

52. The cloning vector of claims 50-51, wherein said homing endonuclease asymmetric recognition site sequences are sequences from 18 to 39 bp.

53. The cloning vector of claim 30, which is linear.

54. The cloning vector of claim 30, wherein RS is replaced by a nucleic acid insert of interest.

55. The cloning vector of claim 54, wherein said insert is selected from the group consisting of DNA, cDNA and RNA/DNA hybrid.

56. The cloning vector of claim 54, wherein said insert is a long cDNA.

57. The cloning vector of claim 54, wherein said insert is a full-length cDNA.

58. The cloning vector of claim 57, wherein said full-length cDNA is a normalized and/or subtracted full-length cDNA.

59. A method for cloning a nucleic acid insert of interest or for preparing a bulk nucleic acid library of interest, comprising the steps of:
(a) preparing at least a bacteriophage cloning vector comprising a construction segment (CS) and a replaceable segment (RS), said RS comprising the ccdB gene;
(a) replacing RS with a nucleic acid insert of interest into the cloning vector;
(c) allowing the in vivo or in vitro excision of the nucleic acid insert of interest or of the plasmid comprising the nucleic acid insert of interest;
(d) recovering the (recombinant) plasmid carrying the nucleic acid insert of interest and lacking the ccdB gene or a library of these plasmids.

60. The method of claim 59, wherein between the steps b) and c) an amplification step of the at least a cloning vector is carried out.

61. A method for cloning a nucleic acid of interest or a bulk nucleic acid library of interest, comprising the step of:
(a) preparing at least bacteriophage cloning vector comprising a construction segment (CS) and a replaceable segment (RS), wherein said RS comprises at least the ccdB gene; wherein RS is flanked by two recombination sites, and said two recombination sites do not recombine with each other;
(b) replacing RS with a nucleic acid insert of interest into the cloning vector obtaining a product according to claims 54-58;
(c) allowing the in vitro excision of the nucleic acid insert of interest by providing to the cloning vector of step b) at least a destination vector comprising a destination replaceable segment (RS) flanked by two recombination sites, said two recombination sites do not recombine with each other, and said destination RS comprises at least the ccdB gene;
(d) recovering a recombinant plasmid carrying the nucleic acid insert of interest and lacking of the ccdB gene or a library of said plasmids.

62. (The method of claim 61, wherein between the steps b) and c) an amplification step of the at least a plasmid is carried out.

63. The method of claim 61, wherein said two recombination sites of both the cloning vector of step a) and the destination vector of step d) are derived from recombination site selected from the group consisting of attB, attP, attL, and attR or derivatives thereof.

64. The method of claim 61, wherein said recombination sites flanking RS are lox recombination sites or derivatives thereof, and do not recombine with each other.

65. The method of claim 64, wherein said lox recombination sites are loxP or derivatives thereof.

66. A method for cloning a nucleic acid insert of interest or for preparing a bulk nucleic acid library of interest, comprising the steps of:
(a) preparing at least a cloning vector comprising a construction segment (CS) and a replaceable segment (RS), said CS comprising two recombination sites which recombine with each other, and said RS comprising a recombination site capable of recombining with one of the two sites placed into CS;
(b) replacing RS with a nucleic acid insert of interest into the cloning vector of step a);
(c) allowing the in vivo or in vitro excision of the nucleic acid insert of interest or of the plasmid comprising the nucleic acid insert of interest;
(d) recovering the (recombinant) plasmid carrying the nucleic acid insert of interest or a library of said plasmids.

67. The method of claim 66, wherein said RS and CS recombination sites are lox recombination site or derivatives thereof

68. The method of claim 67, wherein said lox site is a loxP site or derivatives thereof.

69. A method for cloning a nucleic acid insert of interest or for preparing a bulk nucleic acid library of interest, comprising the steps of:
(a) preparing at least a cloning vector comprising a construction segment (CS) and a replaceable segment (RS), said RS being flanked by two endonuclease asymmetric recognition site sequences, which do not ligate with each other;
(b) replacing RS with a nucleic acid insert of interest comprising two endonuclease asymmetric recognition site sequences flanking said insert of interest, said sequences being capable of ligating with the two sequences placed into the vector of step a), and obtaining a vector comprising the nucleic acid insert of interest;
(c) allowing the in vivo or in vitro excision of the nucleic acid insert of interest or of the plasmid comprising the nucleic acid insert of interest;
(d) recovering the (recombinant) excised plasmid or destination plasmid carrying the nucleic acid insert of interest or a library of said plasmids.

70. The method of claim 69, wherein said endonuclease asymmetric recognition site sequences are: i) two homing endonuclease asymmetric recognition site sequences; or ii) two asymmetric restriction endonuclease cleavage site sequences recognizable by class IIS restriction enzymes.

71. The method of claim 70, wherein said restriction homing endonucleases capable of cutting said asymmetric site sequences are selected from the group consisting of: I-CeuI, PI-Scei, PI-PspI and I-SceI.

72. The method of claims 70, wherein said homing endonuclease asymmetric site sequences are from 18 to 39 bp.

73. A method for cloning a nucleic acid insert of interest or preparing a bulk nucleic acid library of interest comprising the steps of:
(a) preparing at least a cloning vector, comprising a construction segment (CS) and a replaceable segment (RS), wherein said CS is a bacteriophage vector comprising two lox recombination sites or derivatives thereof;
(b) replacing RS with a nucleic acid insert of interest into the cloning vector;
(c) packaging of the vector;
(d) in vivo in liquid-phase infection of at least a cell expressing Cre-recombinase;
(e) allowing the in vivo in liquid-phase excision of at least a plasmid comprising the nucleic acid insert of interest under condition of short-time growth or no growth of the excised plasmid;
(ii.)(f) carrying out cellular lysis and recovery of the plasmid carrying the insert or of a library of said plasmids.

74. The method of claim 63, further comprising the step of:
(g) electroporating or transforming at least a cell, not expressing Cre-recombinase, making the plasmid(s) of step f) penetrating into said cell(s);
(h) plating of cell(s) infected as at step g) and recovering the plasmid carrying the nucleic acid insert of interest or a library of said plasmids.

75. The method of claim 72, wherein said bacteriophage is λ.

76. The method of claim 73, wherein said lox recombination sites are loxP or derivatives thereof.

77. The method of claim 73, wherein between the steps c) and d) an amplification of the packaged vector(s) is carried out.

78. The method of claims 73-77, wherein the cloning vector of step a) is a cloning vector according to claims 1-22 or 30-53, and the product of step b) is a vector comprising the insert of interest according to claims 23-27 or 54-58.

79. The method of claim 73, wherein the step e) is carried out in 0-3 hours at the temperature 20-45° C.

80. A method for cloning a nucleic acid insert of interest or for preparing a bulk nucleic acid library of interest comprising the step of:
(a) preparing at least a cloning vector, comprising a construction segment (CS) and a replaceable segment (RS), wherein said CS is a bacteriophage vector segment comprising two lox recombination sites or derivatives thereof positioned at left and right side of said RS;
(b) replacing RS with a nucleic acid insert of interest into the cloning vector;
(c) in vitro packaging of the at least a bacteriophage cloning vector of step b) in presence of packaging extract;
(d) extraction of bacteriophage cloning vector from the capside;
(e) in vitro excision of the plasmid comprising the nucleic acid insert of interest from the vector in presence of Cre-recombinase;
(f) recovery of said plasmid or library of plasmids.

81. The method of claim 80, further comprising the step:
(g) electroporating or transforming at least a cell, not expressing Cre-recombinase, making said plasmid entering into said cell;
(h) plating the cell of step g) and recovering plasmid carrying the nucleic acid insert of interest or a library of said plasmids.

82. The method of claims 80-81, wherein between the steps c) and d), an amplification step on plate of the bacteriophage is carried out.

83. The method of claim 80, wherein the lox recombination sites are loxP or derivatives thereof.

84. The method of claim 80, wherein said bacteriophage is λ.

85. The method of claims 80-84, wherein the cloning vector of step a) is a cloning vector according to claims 1-22 or 30-53 and the insert of interest of step b) is according to claims 23-27 or 54-58.

86. A bacteriophage cloning vector comprising a construction segment (CS) and a replaceable segment (RS), wherein said RS is flanked by two recombination sites, and said two recombinant sites do not recombine with each other.

87. The cloning bacteriophage vector of claim 86, wherein said bacteriophage is λ.

88. The cloning vector of claims 86-87, wherein said recombination sites are selected from the group consisting of attB, attP, attL, attR and derivatives thereof.

89. The cloning vector of claim 86, wherein CS further comprises two lox recombination sites or derivatives thereof, said lox sites being capable of recombining with each other.

90. The cloning vector of claim 89, wherein said lox recombination sites are loxP or derivatives thereof.

91. The cloning vector of claim 86, wherein the size of the bacteriophage λ vector segment (CS) is: 32 kb≦CS≦45 kb.

92. The cloning vector of claim 91, wherein CS is: 36.5 kb≦CS<38 kb.

93. The cloning vector of claim 91, wherein CS is 37.5 kb.

94. The cloning vector of claim 86, wherein the bacteriophage CS comprises a plasmid segment at least comprising an ori.

95. The cloning vector of claim 94, wherein said plasmid segment comprising an ori is selected from the group consisting of: pBluescript(+), pUC, pBR322 and pBAC.

96. The cloning vector of claim 86, wherein CS further comprises at least a selectable marker selected from the group consisting of: a DNA segment that encodes a product that provides resistance against otherwise toxic compounds; a DNA segment that encodes a product that suppresses the activity of a gene product; a DNA segment that encodes a product that is identifiable; a DNA segment that binds a product that modifies a substrate; a DNA segment that provides for the isolation of a desired molecule; a DNA segment that encodes a specific nucleotide recognition sequence which is recognized by an enzyme.

97. The cloning vector of claim 96, wherein said selectable marker comprises at least a marker selected from the group consisting of an antibiotic resistance gene, an auxotrophic marker, a toxic gene, a phenotypic marker, an enzyme cleavage site, a protein binding site; and a sequence complementary to a PCR primer sequence.

98. The cloning vector of claim 86, wherein RS further comprising at least a background-reducing sequence selected from the group consisting of: i) the ccdB gene, ii) the lacZ gene, iii) a lox sequence.

99. The cloning vector of claim 98, wherein said lox sequence is loxP.

100. The cloning vector of claim 86, wherein RS is flanked by i) two homing endonuclease asymmetric recognition site sequences, which do not ligate with each other; or ii) two asymmetric restriction endonuclease cleavage sites sequences recognizable by class IIS restriction enzymes.

101. The cloning vector of claim 100, wherein said homing endonucleases capable of cutting said asymmetric site sequences are selected from the group consisting of: I-CeuI, PI-SceI, PI-PspI and I-SceI.

102. The cloning vector of claims 100-101, wherein said homing endonuclease asymmetric site sequences are sequences from 18 to 39 bp.

103. The cloning vector of claim 86, which is linear.

104. The cloning vector of claim 86, wherein RS is replaced by a nucleic acid insert of interest.

105. The cloning vector of claim 10, wherein said insert is selected from the group consisting of DNA, cDNA, RNA/DNA hybrid.

106. The cloning vector of claim 104, wherein said insert is a long cDNA.

107. The cloning vector of claim 104, wherein said insert is a full-length cDNA.

108. The cloning vector of claim 107, wherein said full-length cDNA is a normalized and/or subtracted full-length cDNA.

109. A method for cloning a nucleic acid insert of interest or for preparing a bulk nucleic acid library of interest, comprising the steps of:
(a) preparing at least a cloning vector comprising a construction segment (CS) and a replaceable segment (RS), wherein said CS is a bacteriophage vector segment and RS is flanked by two recombination sites, and said two recombinant sites do not recombine with each other;
(b) replacing said RS with a nucleic acid insert and obtaining the product of claims 105-108;
(c) in vitro packaging the at least a bacteriophage cloning vector of step b);
(d) allowing the in vitro excision of the nucleic acid insert(s) of interest by providing to the at least a cloning vector of step c) an at least a destination vector comprising a destination replaceable segment (RS) flanked by two recombination sites, and said two recombination sites do not recombine with each other;
(e) recovering a recombinant plasmid carrying the nucleic acid insert of interest or a library of said plasmids.

110. The method of claim 109, wherein said bacteriophage is λ.

111. The method of claims 109-110, wherein said two recombination sites of both the cloning vector of step a) and the destination vector of step d) are derived from recombination sites selected from the group consisting of attB, attP, attL, attR and derivatives thereof.

112. The method of claim 109, wherein said two recombinant sites of both step a) and step d) are lox recombination sites or derivatives thereof, which do not recombine each other.

113. The method of claim 112, wherein said lox recombination site is loxP or derivative thereof.

114. The method of claim 109, wherein said RS of the destination vector of step d) further comprises at least the ccdB gene

115. The method of claim 109, wherein the CS of the vector cloning further comprises a selectable marker.

116. The method of claim 109, further comprising the steps of:
(f) providing an at least a second destination vector comprising a destination replaceable segment (RS) flanked by two recombination sites, and said two recombination sites do not recombine with each other, in contact with the plasmid(s) of step (e).

117. The method of claim 109, further comprising a step of 1) electroporating at least a cell making the plasmid obtained in step e) or f) entering said cell; and 2) plating the cell of step 1) and recovering of the plasmid or plasmids carrying the insert

118. A kit comprising at least a cloning vector or at least a library of vectors according to claim 1.

119. A method for preparing at least one normalized and/or subtracted library comprising the steps of:
(f) providing at least an excised plasmid or a destination plasmid prepared according to claim 28;
(g) providing the plasmid of step b) to a pool of nucleic acid targets;
(h) removing the hybrids;
(i) collected the normalized and/or subtracted nucleic acid targets.

120. The method of claim 119, wherein the plasmid of step b) is treating by 1) making at least a nick into only one strand of the double stranded plasmid(s); 2) removing the plasmid fragments which have been nicked; 3) collecting the single strand(s) which has not been nicked; 4) applying the steps (c)-(d).

121. The method of claim 120, wherein the nick is introduced by using the GeneII protein.

122. The method of claim 120, wherein the strand which has been nicked is removed by an esonuclease.

123. The method of claim 122, wherein the esonuclease is ExoIII.

124. A method for preparing at least a normalized and/or subtracted library comprising the steps of:
(a) providing at least a cloning bacteriophage vector comprising a construction segment (CS) and a replaceable segment (RS), wherein the size of CS is: X−1.2 kb≦CS<X; wherein X corresponding to the minimum size necessary to the vector for undergoing packaging; wherein the CS of the vector comprises a F1 ori
(b) replacing RS with a nucleic acid insert of interest according to claims 23-27;
(c) adding an helper phage and producing a number of a single strand plasmid vector copies;
(d) providing the copies of step c) to a pool of nucleic acids targets;
(e) removing the hybrids;
(f) collected the normalized and/or subtracted nucleic acid targets.

125. A bacteriophage vector comprising a bacterial artificial chromosome (pBAC) or a segment thereof comprising at least an origin of replication (ori).

126. The bacteriophage of claim 125, wherein the bacteriophage is □.bacteriophage.

127. The bacteriophage of claim 125-126, wherein the pBAC or segment thereof further comprises:
a site into which an DNA fragment can be cloned;
at least one pair of inducible excision-mediating sites flanking the site into which the DNA fragment can be cloned, the excision-mediating sites defining an excisable fragment that comprises the site into which the DNA fragment can be cloned.

128. The bacteriophage of claim 127, wherein the pair of excision-mediating sites are FRT sites.

129. The bacteriophage of claim 127, wherein the pair of excision-mediating sites comprise a sequence as shown in SEQ ID NO:45.

130. The bacteriophage of claim 125, wherein the ori is an ori capable of maintaining the plasmid at single copy.

131. The bacteriophage of claim 125, wherein the pBAC or segment thereof further comprises an inducible origin of replication.

132. The bacteriophage of claim 131, wherein the inducible origin of replication is oriV.

133. The bacteriophage of claims 125-126, comprising a bacterial artificial chromosome (pBAC) or a segment thereof comprising an inducible origin of replication.

134. The bacteriophage of claim 125, comprising at least two recombination sites selected from the following: (a) two recombination sites, wherein either site does not recombine with the other; (b) two lox recombination sites, wherein either site is capable of recombining with each other; (c) two homing endonuclease asymmetric recognition site sequences; (d) two restriction asymmetric endonuclease cleavage site sequences, wherein either site sequence does ligate with the other, recognizable by class IIS restriction enzymes.

135. The bacteriophage of claim 134, wherein the two recombination sites (a) are selected from the group consisting of attB, attP, attL, attR and derivatives thereof.

136. The bacteriophage of claim 134, wherein the two recombination sites (a) are lox recombination sites derivative, which do not recombine with each other.

137. The bacteriophage of claim 134, wherein the two recombination sites (b) are loxp sites.

138. The bacteriophage of claim 134, wherein the two homing endonuclease site sequences (c) are selected from the group consisting of: I-CeuI, PI-SceI, PI-PspI, and I-SceI.

139. The bacteriophage of claim 125, further comprising at least a background-reducing sequence.

140. The bacteriophage of claims 139, wherein the at least background-reducing sequence is selected from: a) the ccdB gene; b) the lacZ gene; c) a lox sequence.

141. A method for cloning a nucleic acid of interest or for preparing a bulk nucleic acid library of interest comprising the steps of:
(a) preparing a bacteriophage cloning vector according to claim 125;
(b) inserting a nucleic acid of interest into the bacteriophage cloning vector;
(c) allowing the in vivo or in vitro excision of the BAC plasmid comprising the nucleic acid insert of interest;
(d) recovering the BAC plasmid carrying the nucleic acid insert of interest or a library of these BAC plasmids.
